(19) United States
(12) Patent Application Publication
(10) Pub. No.: US 2024/0200018 A1
(43) Pub. Date: Jun. 20, 2024

(54) COMPOSITIONS FOR PRESERVING ANAEROBIC MICROORGANISMS AND METHODS OF MAKING AND USING THE SAME

(71) Applicant: Arranta Bio Holdings, LLC, Watertown, MA (US)

(72) Inventor: Aaron Cowley, Watertown, MA (US)

(21) Appl. No.: 18/555,799

(22) PCT Filed: Apr. 29, 2022
(86) PCT No.: PCT/US2022/027058

§ 371 Date: Oct. 17, 2023

Related U.S. Application Data
(60) Provisional application No. 63/182,578, filed on Apr. 30, 2021.

(51) Int. Cl. C12N 1/04; A61K 35/74

(52) U.S. Cl. CPC C12N 1/04; A61K 35/74

(57) ABSTRACT

Provided herein are compositions for preserving one or more species of anaerobic microorganisms. Also provided are preserved compositions of anaerobic microorganisms, e.g., cryopreserved compositions, as well as methods for preparing and using the preserved compositions.

### CROSS-REFERENCE

This application claims the benefit of, and priority to, U.S. Provisional Application No. 63/182,578, filed Apr. 30, 2021, the content of which is hereby incorporated by reference in its entirety.

### FIELD

The disclosure relates generally to methods and compositions for preserving anaerobic microorganisms.

### BACKGROUND

Live biotherapeutic products (LBPs), which contain viable microorganisms, belong to a class of therapies that are being developed for the treatment of a wide variety of medical disorders including, for example, gastrointestinal tract diseases, such as Crohn's disease and ulcerative colitis. It is believed that LBPs exert their therapeutic effect through the modulation of the gastrointestinal environment, including the microbiome.

A common approach for delivering live microorganisms to the gastrointestinal tract is via oral delivery of a LBP. However, significant challenges must be addressed when developing a LBP to ensure that is safe and effective. Such challenges include, but are not limited to, maintaining the stability and viability of the microorganisms during manufacturing, storage, and administration, achieving the desired release profile of the live microorganisms into the gastrointestinal tract, and ensuring that the microorganisms recover and proliferate during passage through appropriate regions of the gastrointestinal tract so as to achieve the desired therapeutic effect.

Cryopreservation is a technique often used for formulating LBPs for oral delivery. Given that live microorganisms, including anaerobic organisms, may have a short, finite life span, the cryopreservation process may facilitate an extended period of time when the microorganisms remain viable, as is required for a LBP. However, the use of cryopreservation in itself can result in a significant reduction in the viability of live microorganisms, often caused by cell death associated with the formation of intra-cellular and/or extra-cellular ice crystals. Furthermore, the stability of cryopreserved formulations may also impact viability during storage, rehydration and timely recovery of the organisms following ingestion by a subject. Although a number of approaches have been developed for protecting microorganisms from damage during the cryopreservation process, including the use of cryoprotectants and lyoprotectants, and the control of the lyophilization processes, these approaches to date often result in LBPs with substantially diminished viability especially over extended periods of time, that require administration of higher closes of organisms in order to achieve the desired therapeutic effect.

Despite the efforts made to date, there remains an unmet need to develop preservative compositions, for example, cryopreservative compositions, that improve the viability, of microorganisms during manufacturing, storage, and administration, and that facilitate the timely recovery of organisms following administration so the organisms can impart a therapeutic effect as they traverse the digestive tract of the subject.

### SUMMARY

The present invention is based, in part, upon the discovery of preservative compositions and preservation approaches that facilitate the long-term viability of anaerobic microorganisms, as well as the rapid recovery of the microorganisms when ingested by a subject so that the organisms can replicate as they traverse the digestive tract of the subject. As generally described herein, the present disclosure provides compositions for preserving anaerobic microorganisms, including compositions for cryopreserving anaerobic microorganisms. The compositions generally comprise a viscosity modifier, an energy source for the microorganisms to facilitate rapid recovery upon administration to a subject and a redox potential to support the viability of anerobic organisms, e.g. from about −50 mV to about −500 mV. In addition, the compositions disclosed herein generally comprise one or more different types of anaerobic microorganisms, a viscosity modifier, an energy source for the microorganisms and a redox potential to support the viability of anerobic organisms, e.g. from about −50 mV to about −500 mV.

In one aspect, the disclosure provides a composition (e.g., a cryopreservative composition) for preserving one or more species of anaerobic microorganisms. The composition comprises: a viscosity modifier, and an energy source for the microorganisms, and, when solvated in an aqueous solvent, has a viscosity in the range of about 5 cP to about 6,000 cP, an osmolality in the range of about 300 Osm/L to about 4,000 Osm/L, a pH in the range of about 4 to about 8, and optionally a redox potential in the range of −50 mV to −500 mV.

In another aspect, the disclosure provides a cryopreservative composition for cryopreserving one or more species of anaerobic microorganisms. The composition comprises: a viscosity modifier, an energy source for the microorganisms, and, when solvated in an aqueous solvent, has a viscosity in the range of about 5 cP to about 6,000 cP, an osmolality in the range of about 300 Osm/L to about 4,000 Osm/L, a pH in the range of about 4 to about 8, and optionally a redox potential in the range of −50 mV to −500 mV.

In each of the foregoing aspects, the composition has a viscosity is in the range of about 50 cP to about 4,000 cP, about 100 cP to about 2,000 cP, or about 100 cP to about 1,000 cP and/or an osmolality in the range of about 300 Osm/L to about 3,000 Osm/L, about 400 Osm/L to about 3,000 Osm/L, or about 500 Osm/L to about 3,000 Osm/L and/or a pH in the range of about 4 to about 8, about 5 to about 8, about 4 to about 7.5, about 5 to about 7.5, or about 5.5 to about 7.5.

In each of the foregoing aspects, the composition the composition can contain from about 0.01% (w/w) to about 0.4% (w/w) of the viscosity modifier, when solvated in an aqueous solvent. The viscosity modifier can be a polymeric composition, for example, a composition comprising an alginate, acacia, alginic acid, an agar, alamic acid, bentonite, carbomer, a carrageenan, a cellulose, a dextrin, a gelatin, gellan gum, guar gum, a carboxymethylcellulose, a hydroxyethylcellulose, a hydroxypropylcellulose, a methylcellulose, a mucin, pectin, a starch, xanthan gum or a combination thereof. In certain embodiments, the polymeric composition comprises an alginate, for example, sodium alginate having, for example, a viscosity range of about 250 cP to about 14,000 cP.

Furthermore, in each of the foregoing compositions, the energy source can be a cell culture media that permits growth of the microorganisms. In certain embodiments, the composition comprises about 3% (w/w) to about 150% (w/w) of the energy source, when solvated in an aqueous solvent. The energy source can be a monosaccharide, a disaccharide, an oligosaccharide, or a combination thereof. The monosaccharide can be, for example, selected from the group consisting of glucose, fructose, xylose, and combinations thereof. The disaccharide can be, for example, trehalose, sucrose, or a combination thereof. The oligosaccharide can be, for example, an inulin derivative, for example, a fructo-oligosaccharide.

Furthermore, in each of the foregoing compositions, the composition can further comprise a protein stabilizer. In certain embodiments, the composition comprises about 5% (w/w) to about 10% (w/w) of the protein stabilizer, when solvated in an aqueous solvent. The protein stabilizer can be selected from the group consisting of an amino acid, an antioxidant, a disaccharide, a reducing agent, polyethylene glycol, and combinations thereof. In certain embodiments, the disaccharide is selected from the group consisting of trehalose, sucrose, isomaltose, lactose, maltose, lactulose, cellobiose, and combinations thereof.

In certain embodiments, the one or more anaerobic microorganisms (the organisms can belong to different genera or species) are present in the form of a cell paste or cell slurry. Depending upon the circumstances, in each of the foregoing compositions, the composition can comprise about 30% (w/w) to about 50% (w/w) of a cell paste or cell slurry, when solvated in an aqueous solvent.

In another aspect, the disclosure provides a cryopreservative composition for cryopreserving anaerobic microorganisms. The composition comprises: (i) about 30% (w/w) to about 50% (w/w) of a cell paste or cell slurry, wherein the cell paste or slurry comprises one or more anaerobic microorganisms; (ii) about 3% (w/w) to about 15% (w/w) of an energy source for the microorganisms; (iii) about 0.01% (w/w) to about 0.4% (w/w) of a viscosity modifier; and (iv) about 30% (w/w) to about 60% (w/w) of an aqueous solvent, wherein the cryopreservative composition has a viscosity in the range of about 5 cP to about 6,000 cP, an osmolality in the range of about 300 Osm/L to about 4,000 Osm/L, and a pH in the range of about 4 to about 8.

In another aspect, the disclosure provides a cryopreservative composition for cryopreserving anaerobic microorganisms. The composition comprises: (i) about 30% (w/w) to about 50% (w/w) of a cell paste, wherein the cell paste comprises one or more anaerobic microorganisms; (ii) about 0.5% (w/w) to about 5% (w/w) raftilose; (iii) about 3% (w/w) to about 10% (w/w) maltodextrin: (iv) about 0.01% (w/w) to about 0.4% (w/w) sodium alginate; (v) about 5% (w/w) to about 10% (w/w) trehalose; (vi) 0% (w/w) to about 5% (w/w) sucrose; and (vii) about 35% (w/w) to about 55% (w/w) water, wherein the cryopreservative composition has a viscosity in the range of about 5 cP to about 6,000 cP, an osmolality in the range of about 300 Osm/L to about 4,000 Osm/L, and a pH in the range of about 4 to about 8. The composition can have a redox potential in the range of −50 mV to −500 mV.

In each of the foregoing compositions, the composition can also comprise a buffering agent.

In addition, the disclosure provides a preserved cellular composition (for example, a cryopreserved composition) comprising: one or more anaerobic microorganisms, a viscosity modifier, an energy source for the microorganisms, a redox potential in the range of −50 mV to 500 mV, and a water content of less than 5% (w/w). In certain embodiments, the water content is in the range of about 2% (w/w) to about 4% (w/w) or about 2% (w/w) to about 3% (w/w). In certain embodiments, the viscosity modifier is a polymeric composition, for example, an alginate, acacia, alginic acid, an agar, alamic acid, bentonite, carbomer, a carrageenan, a cellulose, a dextrin, a gelatin, gellan gum, guar gum, a carboxymethylcellulose, a hydroxyethylcellulose, a hydroxypropylcellulose, a methylcellulose, a mucin, pectin, a starch, xanthan gum or a combination thereof. In certain embodiments, the polymeric composition comprises an alginate, for example, sodium alginate, for example, sodium alginate having a viscosity range from 250 to 14,000 cP. In certain embodiments, the energy source is a cell culture media that permits growth of the microorganisms. The energy source can be a monosaccharide, a disaccharide, an oligosaccharide, or a combination thereof. The monosaccharide can be, for example, selected from the group consisting of glucose, fructose, xylose, and combinations thereof. The disaccharide can be, for example, selected from the group consisting of trehalose, isomaltose, lactose, maltose, lactulose, cellobiose, sucrose, and combinations thereof. The oligosaccharide can be, for example, an inulin derivative, for example, a fructo-oligosaccharide.

In certain embodiments, the composition comprises a protein stabilizer. The protein stabilizer can be selected from the group consisting of an amino acid, an antioxidant, a disaccharide, a reducing agent, polyethylene glycol, and combinations thereof. The disaccharide can be, for example, selected from the group consisting of trehalose, sucrose, isomaltose, lactose, maltose, lactulose, cellobiose, and combinations thereof.

In certain embodiments, the comprises a buffering agent.

In another aspect, the disclosure provides a cryopreserved composition comprising: (i) one or more anaerobic microorganisms; (ii) an energy source comprising, for example, raftilose and maltodextrin; (iii) sodium alginate; (iv) a protein stabilizer comprising, for example, trehalose, sucrose, and combinations thereof; and (vi) a water content of less than 5% (w/w). The composition can further comprise a redox potential in the range of −50 mV to −500 mV.

In each of the foregoing compositions, the one or more anaerobic microorganisms can be selected from the group consisting of Akkermansiaceae, Bacillaceae, Bacteroidaceae, Bifidobacteriaceae, Christensenellaceae, Clostridiaceae, Coriobacteriaceae, Eggerthellaceae, Enterobacteriaceae, Eubacteriaceae, Lachnospiraceae, Lactobacillaceae, Leuconostocaceae, Odoribacteraceae, Oxalobacteraceae, Porphyromonadaceae, Prevotellaceae, Rikenellaceae, Ruminococcaceae, Streptococcaceae, and combinations thereof.

In certain embodiments, the cellular compositions described herein, when stored at 4° C. for 6 months or 12 months and then rehydrated results in a loss of colony forming units (CFUs) by less than 3, 2, 1 or 0.5 log. In certain embodiments, the composition remains substantially stable at 4° C. for about 2 years. In certain embodiments, the composition, when rehydrated in an aqueous fluid, permits regrowth of the one or more microorganisms within 5, 4, 3, or 2 hours of rehydration.

In addition, the disclosure provides a pharmaceutical composition comprising the cryopreserved composition of any one of the cell containing compositions described herein, optionally disposed within a capsule, tablet or caplet. In addition, the disclosure provides a food product or probiotic comprising the cryopreserved composition of any one of the cell containing compositions described herein, optionally disposed within a capsule, tablet or caplet.

In another aspect, the disclosure provides a method of modulating the gastrointestinal microbiome of a subject in need thereof, the method generally comprises administering to the subject an effective amount of any one of the compositions, pharmaceutical compositions, or food products or probiotics described herein.

In another aspect, the disclosure provides a method of producing the cryopreserved composition described herein. The method generally comprises the steps of: (a) combining anaerobic microorganisms with an excipient blend comprising an aqueous mixture comprising a viscosity modifier, and an energy source for the microorganisms to produce a cryopreservative mixture, wherein the cryopreservative mixture has a viscosity in the range of about 5 cP to about 6,000 cP, an osmolality in the range of about 300 Osm/L to about 4,000 Osm/L, a pH in the range of about 4 to about 8, and optionally a redox potential in the range of −50 mV to −500 mV; and (b) freeze drying the cryopreservative mixture to reduce the water content of the cryopreservative mixture to less than about 5% (w/w) to produce the cryopreserved composition. The microorganisms can be in the form of a cell paste, where, for example, the cell paste can comprise the microorganisms disposed within cell culture media. The method may further comprise packaging the cryopreserved composition in a capsule, tablet or caplet.

### BRIEF DESCRIPTION OF THE DRAWINGS

FIG. 1 is a graph showing exemplary temperature and pressure profiles that can be used with an exemplary composition described herein to preserve anaerobic microorganisms disclosed therein.

FIG. 2 is a bar graph showing the viabilities of Rikenellaceae, Bacteroidaceae, Eubacteriaceae, Lachnospiraceae, Lactobacillaceae, Leuconostocaceae, Oxalobacteraceae, and Streptococcaceae microorganisms following cryopreservation in (i) exemplary cryopreservative compositions of the present disclosure (ACP-001, ACP-002 and ACP-003) and (ii) commercially available cryopreservative compositions (CB-001 and CB-002; OPS Diagnostics Cat. No. LR2X-500-02 and MFDB-500-06, as described in Example 4.

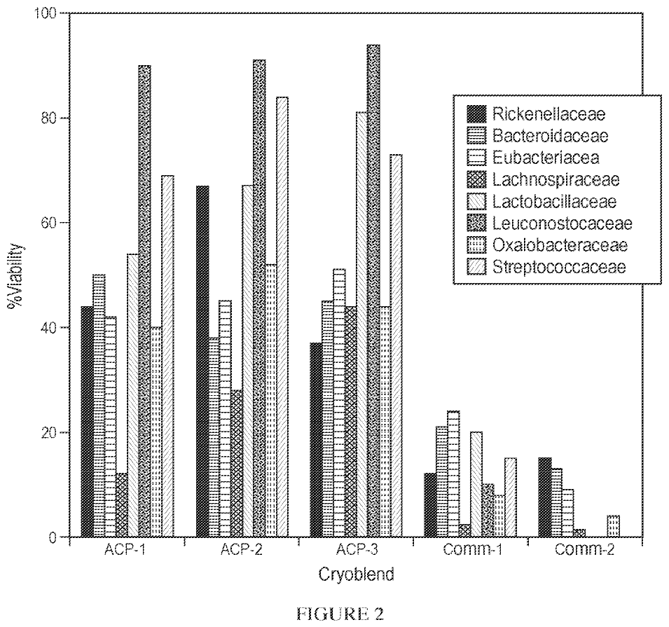

FIG. 3 is a graph shoving the optical density (OD₆₀₀) as a function of ETF (hours) for Bacteroidaceae and Lachnospiraceae cryopreserved compositions, preserved using (i) exemplary cryopreservative compositions of the present disclosure and (ii) commercially available cryopreservative compositions (OPS Diagnostics Cat. No. LR2X-500-02 and MFDB-500-06), as described in Example 4.

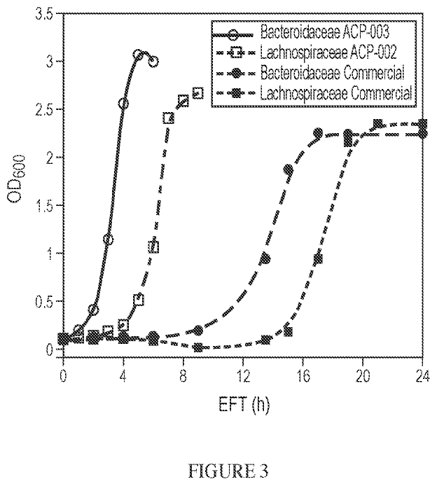

### DETAILED DESCRIPTION

The present invention is based, in part, upon the discovery of preservative compositions and preservation approaches that facilitate the long-term viability of anaerobic microorganisms, as well as the rapid recovery of the microorganisms when ingested by a subject so that the organisms can replicate as they traverse the digestive tract of the subject. As generally described herein, the present disclosure provides compositions for preserving anaerobic microorganisms, including compositions for cryopreserving anaerobic microorganisms. The compositions generally comprise a viscosity modifier, an energy source for anaerobic microorganisms to facilitate rapid recovery upon administration to a subject and a redox potential to support the viability of anerobic organisms, e.g. from about −50 mV to about −500 mV. In addition, the compositions disclosed herein generally comprise one or more different types of anaerobic microorganisms, a viscosity modifier, an energy source for the microorganisms and a redox potential to support the viability of anerobic organisms, e.g. from about −50 mV to about −500 mV.

### Definitions

To facilitate an understanding of the present disclosure, a number of terms and phrases are defined below.

Unless defined otherwise, all technical and scientific terms used herein have the same meaning as commonly understood by one of ordinary skill in the art to which this disclosure belongs. The abbreviations used herein have their conventional meaning within the chemical and biological arts.

Throughout the description, where compositions and kits are described as having, including, or comprising specific components, or where processes and methods are described as having, including, or comprising specific steps, it is contemplated that, additionally, there are compositions and kits of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure(s). For example, it will be appreciated that all features described and depicted herein can be applicable to all aspects of the disclosure(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±20% variation from the nominal value unless otherwise indicated or inferred from the context.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, “pharmaceutical composition” or “pharmaceutical formulation” refers to the combination of an active agent with an excipient, inert or active, making the composition or formulation especially suitable for diagnostic or therapeutic use in vivo or ex vivo.

“Pharmaceutically acceptable” means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable excipient” refers to a substance that aids the administration of an active agent to and/or absorption by a subject and can be included in the compositions of the present disclosure without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, am lose or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. For examples of excipients, see Martin, REMINGTON'S PHARMACEUTICAL SCIENCES, 15th Ed., Mack Publ. Co., Easton, PA (1975).

The terms “subject” and “patient” are used interchangeably herein. A “subject” or “patient” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, “administering” means oral administration, administration as a suppository, topical contact, intravenous administration, parenteral administration, intraperitoneal administration, intramuscular administration, intralesional administration, intrathecal administration, intracranial administration, intranasal administration or subcutaneous administration, or the implantation of a slow-release device, e.g., a mini-osmotic pump, to a subject. Administration can be by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. In certain embodiments, a preserved composition described herein (e.g., a cryopreserved composition) is orally administered to the subject. By “co-administer” it is meant that a composition described herein is administered at the same time, just prior to, or just after the administration of one or more additional therapies. A preserved composition described herein (e.g., a cryopreserved composition) can be administered alone or can be co-administered to the subject. Co-administration is meant to include simultaneous or sequential administration of the composition individually or in combination (more than one composition or agent). Thus, the preparations can also be combined, when desired, with other active substances/compositions.

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (e.g., “therapeutic treatment”).

In general, an “effective amount” of a composition (e.g. a preserved composition or cryopreserved composition described herein) refers to an amount sufficient to elicit the desired biological response, e.g. to modulate the gastrointestinal microbiome of a subject. As will be appreciated by those of ordinary skill in this art, the effective amount of a composition of the disclosure (e.g. a preserved composition or cryopreserved composition described herein) may vary depending on such factors, for example, as the desired biological endpoint, the residence time and viability of the composition, the disease being treated, and the age, weight, health, and condition of the subject.

As used herein, the term “anaerobic microorganism” is understood to mean a microorganism that remains viable and/or is capable of reproducing in an environment depleted of oxygen. The term anaerobic microorganisms includes obligate anaerobes, facultative anaerobes and aerotolerant microorganisms. In certain embodiments, an anaerobic microorganism remains viable and capable of growing and/or replicating in an environment with a redox potential from about +300 mV to −100 mV for facultative anaerobes and +100 mV to about −500 mV for obligate anaerobes.

### I. Compositions for Preserving Anaerobic Microorganisms

In one aspect, provided herein are compositions for preserving anaerobic microorganisms, which generally comprise a viscosity modifier and an energy source for the microorganisms. In certain embodiments, a composition for preserving anaerobic microorganisms described herein is a cryopreservative composition for cryopreserving anaerobic microorganisms.

In certain embodiments, the composition has a viscosity in the range of about 5 cP to about 6,000 cP, when solvated in an aqueous solvent. Alternatively or in addition, the composition has an osmolality in the range of about 300 Osm/L to about 4,000 Osm/L, a redox potential in the range of about −50 mV to about −500 mV, and/or a pH in the range of about 4 to about 8, when solvated in an aqueous solvent.

In certain embodiments, the preservative (e.g., cryopreservative) composition for preserving anaerobic microorganisms comprises a viscosity modifier and an energy source for the microorganisms and optionally (i) a redox potential in the range of about −50 mV to about −500 mV, and (ii) when solvated in an aqueous solvent, has a viscosity in the range of about 5 cP to about 6,000 cP, an osmolality in the range of about 300 Osm/L to about 4,000 Osm/L, a redox potential in the range of about −50 mV to about −500 mV, and a pH in the range of about 4 to about 8.

In certain embodiments, the cryopreservative composition for cryopreserving anaerobic microorganisms comprises a viscosity modifier, an energy source for the microorganisms, and redox potential in the range of about −50 mV to about −500 mV and, when solvated in an aqueous solvent, has a viscosity in the range of about 5 cP to about 6,000 cP, an osmolality, in the range of about 300 Osm/L to about 4,000 Osm/L, a redox potential in the range of about −50 mV to about −500 mV, and a pH in the range of about 4 to about 8.

### Viscosity & Viscosity Modifiers

The viscosity is selected to maintain the viability of the microorganism or microorganisms being preserved.

In certain embodiments, the composition has a viscosity in the range of about 5 cP to about 6,000 cP, about 50 cP to about 6,000 cP, about 100 cP to about 6,000 cP, about 200 cP to about 6,000 cP, about 300 cP to about 6,000 cP, about 400 cP to about 6,000 cP, about 500 cP to about 6,000 cP, about 1,000 cP to about 6,000 cP, about 1,500 cP to about 6,000 cP, about 2,000 cP to about 6,000 cP, about 2,500 cP to about 6,000 cP, about 3,000 cP to about 6,000 cP, about 3,500 cP to about 6,000 cP, about 4,000 cP to about 6,000 cP, about 4,500 cP to about 6,000 cP, about 5,000 cP to about 6,000 cP, about 5,500 cP to about 6,000 cP, about 5 cP to about 5,500 cP, about 5 cP to about 5,000 cP, about 5 cP to about 5,000 cP, about 5 cP to about 4,500 cP, about 5 cP to about 4,000 cP, about 5 cP to about 3,500 cP, about 5 cP to about 3,000 cP, about 5 cP to about 2,500 cP, about 5 cP to about 2,000 cP, about 5 cP to about 1,500 cP, about 5 cP to about 1,000 cP, about 5 cP to about 500 cP, about 5 cP to about 400 cP, about 5 cP to about 300 cP, about 5 cP to about 200 cP, about 5 cP to about 100 cP, about 5 cP to about 50 cP, about 50 cP to about 5,500 cP, about 50 cP to about 5,000 cP, about 50 cP to about 4,500 cP, about 50 cP to about 4,000 cP, about 50 cP to about 3,500 cP, about 50 cP to about 3,000 cP, about 50 cP to about 2,500 cP, about 50 cP to about 2,000 cP, about 50 cP to about 1,500 cP, about 50 cP to about 1,000 cP, about 50 cP to about 500 cP, about 50 cP to about 400 cP, about 50 cP to about 300 cP, about 50 cP to about 200 cP, about 50 cP to about 100 cP, about 100 cP to about 5,500 cP, about 100 cP to about 5,000 cP, about 100 cP to about 4,500 cP, about 100 cP to about 4,000 cP, about 100 cP to about 3,500 cP, about 100 cP to about 3,000 cP, about 100 cP to about 2,500 cP, about 100 cP to about 2,000 cP, about 100 cP to about 1,500 cP, about 100 cP to about 1,000 cP, about 100 cP to about 500 cP, about 100 cP to about 400 cP, about 100 cP to about 300 cP, about 100 cP to about 200 cP, about 200 cP to about 5,500 cP, about 200 cP to about 5,000 cP, about 200 cP to about 4,500 cP, about 200 cP to about 4,000 cP, about 200 cP to about 3,500 cP, about 200 cP to about 3,000 cP, about 200 cP to about 2,500 cP, about 200 cP to about 2,000 cP, about 200 cP to about 1,500 cP, about 200 cP to about 1,000 cP, about 200 cP to about 500 cP, about 200 cP to about 400 cP, about 200 cP to about 300 cP, about 300 cP to about 5,500 cP, about 300 cP to about 5,000 cP, about 300 cP to about 4,500 cP, about 300 cP to about 4,000 cP, about 300 cP to about 3,500 cP, about 300 cP to about 3,000 cP, about 300 cP to about 2,500 cP, about 300 cP to about 2,000 cP, about 300 cP to about 1,500 cP, about 300 cP to about 1,000 cP, about 300 cP to about 500 cP, about 300 cP to about 400 cP, about 400 cP to about 5,500 cP, about 400 cP to about 5,000 cP, about 400 cP to about 4,500 cP, about 400 cP to about 4,000 cP, about 400 cP to about 3,500 cP, about 400 cP to about 3,000 cP, about 400 cP to about 2,500 cP, about 400 cP to about 2,000 cP, about 400 cP to about 1,500 cP, about 400 cP to about 1,000 cP, about 400 cP to about 500 cP, about 500 cP to about 5,500 cP, about 500 cP to about 5,000 cP, about 500 cP to about 4,500 cP, about 500 cP to about 4,000 cP, about 500 cP to about 3,500 cP, about 500 cP to about 3,000 cP, about 500 cP to about 2,500 cP, about 500 cP to about 2,000 cP, about 500 cP to about 1,500 cP, about 500 cP to about 1,000 cP, about 1,000 cP to about 5,500 cP, about 1,000 cP to about 5,000 cP, about 1,000 cP to about 4,500 cP, about 1,000 cP to about 4,000 cP, about 1,000 cP to about 3,500 cP, about 1,000 cP to about 3,000 cP, about 1,000 cP to about 2,500 cP, about 1,000 cP to about 2,000 cP, about 1,000 cP to about 1,500 cP, about 1,500 cP to about 5,500 cP, about 1,500 cP to about 5,000 cP, about 1,500 cP to about 4,500 cP, about 1,500 cP to about 4,000 cP, about 1,500 cP to about 3,500 cP, about 1,500 cP to about 3,000 cP, about 1,500 cP to about 2,500 cP, about 1,500 cP to about 2,000 cP, about 2,000 cP to about 5,500 cP, about 2,000 cP to about 5,000 cP, about 2,000 cP to about 4,500 cP, about 2,000 cP to about 4,000 cP, about 2,000 cP to about 3,500 cP, about 2,000 cP to about 3,000 cP, about 2,000 cP to about 2,500 cP, about 2,500 cP to about 5,500 cP, about 2,500 cP to about 5,000 cP, about 2,500 cP to about 4,500 cP, about 2,500 cP to about 4,000 cP, about 2,500 cP to about 3,500 cP, about 2,500 cP to about 3,000 cP, about 3,000 cP to about 5,500 cP, about 3,000 cP to about 5,000 cP, about 3,000 cP to about 4,500 cP, about 3,000 cP to about 4,000 cP, about 3,000 cP to about 3,500 cP, about 3,500 cP to about 5,500 cP, about 3,500 cP to about 5,000 cP, about 3,500 cP to about 4,500 cP, about 3,500 cP to about 4,000 cP, about 4,000 cP to about 5,500 cP, about 4,000 cP to about 5,000 cP, about 4,000 cP to about 4,500 cP, about 4,500 cP to about 5,500 cP, about 4,500 cP to about 5,000 cP, or about 5,000 cP to about 5,500 cP, when solvated in an aqueous solvent.

In certain embodiments, the viscosity is in the range of from about 50 cP to about 4,000 cP, from about 100 cP to about 2,000 cP, or from about 100 cP to 1,000 cP, when solvated in an aqueous solvent.

In certain embodiments, the composition has a viscosity of about 5 cP, about 10 cP, about 15 cP, about 20 cP, about 25 cP, about 30 cP, about 35 cP, about 40 cP, about 45 cP, about 50 cP, about 55 cP, about 60 cP, about 65 cP, about 70 cP, about 75 cP, about 80 cP, about 85 cP, about 90 cP, about 95 cP, about 100 cP, about 110 cP, about 120 cP, about 130 cP, about 140 cP, about 150 cP, about 160 cP, about 170 cP, about 180 cP, about 190 cP, about 20 cP, about 225 cP, about 250 cP, about 275 cP, about 300 cP, about 350 cP, about 400 cP, about 450 cP, about 500 cP, about 600 cP, about 700 cP, about 800 cP, about 900 cP, about 1,000 cP, about 1,250 cP, about 1,500 cP, about 2,000 cP, about 2,500 cP, about 3,000 cP, about 3,500 cP, about 4,000 cP, about 4,500 cP, about 5,000 cP, about 5,500 cP, about 6,000 cP, when solvated in an aqueous solvent.

In certain embodiments, the composition comprises about 0.01% (w/w) to about 0.4% (w/w), about 0.05% (w/w) to about 0.4% (w/w), about 0.1% (w/w) to about 0.4% (w/w), about 0.15% (w/w) to about 0.4% (w/w), about 0.2% (w/w) to about 0.4% (w/w), about 0.25% (w/w) to about 0.4% (w/w), about 0.3% (w/w) to about 0.4% (w/w), about 0.35% (w/w) to about 0.4% (w/w), about 0.01% (why) to about 0.35% (w/w), about 0.01% (w/w) to about 0.3% (w/w), about 0.01% (why) to about 0.25% (w/w), about 0.01% (w/w) to about 0.2% (w/w), about 0.01% (w/w) to about 0.15% (w/w), about 0.01% (w/w) to about 0.1% (why), about 0.01% (w/w) to about 0.05% (w/w), about 0.05% (w/w) to about 0.35% (w/w), about 0.05% (w/w) to about 0.3% (w/w), about 0.05% (w/w) to about 0.25% (w/w), about 0.05% (w/w) to about 0.2% (w/w), about 0.05% (w/w) to about 0.15% (w/w), about 0.05% (w/w) to about 0.1% (w/w), about 0.1% (w/w) to about 0.35% (w/w), about 0.1% (w/w) to about 0.3% (w/w), about 0.1% (w/w) to about 0.25% (w/w), about 0.1% (w/w) to about 0.2% (w/w), about 0.1% (w/w) to about 0.15% (w/w), about 0.15% (w/w) to about 0.35% (w/w), about 0.15% (w/w) to about 0.3% (w/w), about 0.15% (w/w) to about 0.25% (w/w), about 0.15% (w/w) to about 0.2% (w/w), about 0.2% (w/w) to about 0.35% (w/w), about 0.2% (w/w) to about 0.3% (w/w), about 0.2% (w/w) to about 0.25% (w/w), about 0.25% (w/w) to about 0.35% (w/w), about 0.25% (w/w) to about 0.3% (w/w), or about 0.3% (w/w) to about 0.35% (w/w) of the viscosity modifier, when solvated in an aqueous solvent. In certain embodiments, the composition comprises about 0.01% (w/w) to about 0.4% (w/w) of the viscosity modifier, when solvated in an aqueous solvent.

In certain embodiments, the composition comprises about 0.01% (w/w), about 0.02% (w/w), about 0.03% (w/w), about 0.04% (w/w), about 0.05% (w/w), about 0.06% (w/w), about 0.07% (w/w), about 0.08% (w/w), about 0.09% (w/w), about 0.1% (w/w), about 0.11% (w/w), about 0.12% (w/w), about 0.13% (w/w), about 0.14% (w/w), about 0.15% (w/w), about 0.16% (w/w), about 0.17% (w/w), about 0.18% (w/w), about 0.19% (w/w), about 0.2% (w/w), about 0.21% (w/w), about 0.22% (w/w), about 0.23% (w/w), about 0.24% (w/w), about 0.25% (w/w), about 0.26% (w/w), about 0.27% (w/w), about 0.28% (w/w), about 0.29% (w/w), about 0.3% (w/w), about 0.31% (w/w), about 0.32% (w/w), about 0.33% (w/w), about 0.34% (w/w), about 0.35% (w/w), about 0.36% (w/w), about 0.37% (w/w), about 0.38% (w/w), about 0.39% (w/w), or about 0.4% (w/w), of the viscosity modifier. Mien solvated in an aqueous solvent.

In certain embodiments, the viscosity modifier is a polymeric composition. In certain embodiments, the polymeric composition comprises an alginate, acacia, alginic acid, an agar, alamic acid, bentonite, carbomer, a carrageenan, a cellulose, a dextrin, a gelatin, gellan gum, guar gum, a carboxymethylcellulose, a hydroxyethylcellulose, a hydroxypropylcellulose, a methylcellulose, a mucin, pectin, a starch, xanthan gum, or a combination thereof. In certain embodiments, the polymeric composition comprises an alginate, for example, sodium alginate.

In certain embodiments, the sodium alginate has a viscosity in the range of about 250 cP to about 14,000 cP, about 500 cP to about 14,000 cP, about 750 cP to about 14,000 cP, about 1,000 cP to about 14,000 cP, about 2,000 cP to about 14,000 cP, about 3,000 cP to about 14,000 cP, about 4,000 cP to about 14,000 cP, about 5,000 cP to about 14,000 cP, about 6,000 cP to about 14,000 cP, about 7,000 cP to about 14,000 cP, about 8,000 cP to about 14,000 cP, about 9,000 cP to about 14,000 cP, about 10,000 cP to about 14,000 cP, about 11,000 cP to about 14,000 cP, about 12,000 cP to about 14,000 cP, about 13,000 cP to about 14,000 cP, about 250 cP to about 13,000 cP, about 250 cP to about 12,000 cP, about 250 cP to about 11,000 cP, about 250 cP to about 10,000) cP, about 250 cP to about 9,000 cP, about 250 cP to about 8,000 cP, about 250 cP to about 7,000 cP, about 250 cP to about 6,000 cP, about 250 cP to about 5,000 cP, about 250 cP to about 4,000 cP, about 250 cP to about 3,000 cP, about 250 cP to about 2,000 cP, about 250 cP to about 1,000 cP, about 250 cP to about 750 cP, about 250 cP to about 500 cP, about 500 cP to about 13,000 cP, about 500 cP to about 12,000 cP, about 500 cP to about 11,000 cP, about 500 cP to about 10,000 cP, about 500 cP to about 9,000 cP, about 500 cP to about 8,000 cP, about 500 cP to about 7,000 cP, about 500 cP to about 6,000 cP, about 500 cP to about 5,000 cP, about 500 cP to about 4,000 cP, about 500 cP to about 3,000 cP, about 500 cP to about 2,000 cP, about 500 cP to about 1,000 cP, about 500 cP to about 750 cP, about 750 cP to about 13,000 cP, about 750 cP to about 12,000 cP, about 750 cP to about 11,000 cP, about 750 cP to about 10,000 cP, about 750 cP to about 9,000 cP, about 750 cP to about 8,000 cP, about 750 cP to about 7,000 cP, about 750 cP to about 6,000 cP, about 750 cP to about 5,000 cP, about 750 cP to about 4,000 cP, about 750 cP to about 3,000 cP, about 750 cP to about 2,000 cP, about 750 cP to about 1,000 cP, about 1,000 cP to about 13,000 cP, about 1,000 cP to about 12,000 cP, about 1,000 cP to about 11,000 cP, about 1,000 cP to about 10,000 cP, about 1,000 cP to about 9,000 cP, about 1,000 cP to about 8,000 cP, about 1,000 cP to about 7,000 cP, about 1,000 cP to about 6,000 cP, about 1,000 cP to about 5,000 cP, about 1,000 cP to about 4,000 cP, about 1,000 cP to about 3,000 cP, about 1,000 cP to about 2,000 cP, about 2,000 cP to about 13,000 cP, about 2,000 cP to about 12,000 cP, about 2,000 cP to about 11,000 cP, about 2,000 cP to about 10,000 cP, about 2,000 cP to about 9,000 cP, about 2,000 cP to about 8,000 cP, about 2,000 cP to about 7,000 cP, about 2,000 cP to about 6,000 cP, about 2,000 cP to about 5,000 cP, about 2,000 cP to about 4,000 cP, about 2,000 cP to about 3,000 cP, about 3,000 cP to about 13,000 cP, about 3,000 cP to about 12,000 cP, about 3,000 cP to about 11,000 cP, about 3,000 cP to about 10,000 cP, about 3,000 cP to about 9,000 cP, about 3,000 cP to about 8,000 cP, about 3,000 cP to about 7,000 cP, about 3,000 cP to about 6,000 cP, about 3,000 cP to about 5,000 cP, about 3,000 cP to about 4,000 cP, about 4,000 cP to about 13,000 cP, about 4,000 cP to about 12,000 cP, about 4,000 cP to about 11,000 cP, about 4,000 cP to about 10,000 cP, about 4,000 cP to about 9,000 cP, about 4,000 cP to about 8,000 cP, about 4,000 cP to about 7,000 cP, about 4,000 cP to about 6,000 cP, about 4,000 cP to about 5,000 cP, about 5,000 cP to about 13,000 cP, about 5,000 cP to about 12,000 cP, about 5,000 cP to about 11,000 cP, about 5,000 cP to about 10,000 cP, about 5,000 cP to about 9,000 cP, about 5,000 cP to about 8,000 cP, about 5,000 cP to about 7,000 cP, about 5,000 cP to about 6,000 cP, about 6,000 cP to about 13,000 cP, about 6,000 cP to about 12,000 cP, about 6,000 cP to about 11,000 cP, about 6,000 cP to about 10,000 cP, about 6,000 cP to about 9,000 cP, about 6,000 cP to about 8,000 cP, about 6,000 cP to about 7,000 cP, about 7,000 cP to about 13,000 cP, about 7,000 cP to about 12,000 cP, about 7,000 cP to about 11,000 cP, about 7,000 cP to about 10,000 cP, about 7,000 cP to about 9,000 cP, about 7,000 cP to about 8,000 cP, about 8,000 cP to about 13,000 cP, about 8,000 cP to about 12,000 cP, about 8,000 cP to about 11,000 cP, about 8,000 cP to about 10,000 cP, about 8,000 cP to about 9,000 cP, about 9,000 cP to about 13,000 cP, about 9,000 cP to about 12,000 cP, about 9,000 cP to about 11,000 cP, about 9,000 cP to about 10,000 cP, about 10,000 cP to about 13,000 cP, about 10,000 cP to about 12,000 cP, about 10,000 cP to about 11,000 cP, about 11,000 cP to about 13,000 cP, about 11,000 cP to about 12,000 cP, or about 12,000 cP to about 13,000 cP. In certain embodiments, the sodium alginate has a viscosity range of about 250 cP to about 14000 cP.

### Osmolality and Osmolality Modifiers

The osmolality is selected to maintain the viability of the microorganism or microorganisms being preserved.

In certain embodiments, the composition has an osmolality in the range of about 300 Osm/L to about 4,000 Osm/L, about 400 Osm/L to about 4,000 Osm/L, about 500 Osm/L to about 4,000 Osm/L, about 600 Osm/L to about 4,000 Osm/L, about 700 Osm/L to about 4,000 Osm/L, about 800 Osm/L to about 4,000 Osm/L, about 900 Osm/L to about 4,000 Osm/L, about 1,000 Osm/L to about 4,000 Osm/L, about 1,500 Osm/L to about 4,000 Osm/L, about 2,000 Osm/L to about 4,000 Osm/L, about 2,500 Osm/L to about 4,000 Osm/L, about 3,000 Osm/L to about 4,000 Osm/L, about 3,500 Osm/L to about 4,000 Osm/L, about 300 Osm/L to about 3,500 Osm/L, about 300 Osm/L to about 3,000 Osm/L, about 300 Osm/L to about 2,500 Osm/L, about 300 Osm/L to about 2,000 Osm/L, about 300 Osm/L to about 1,500 Osm/L, about 300 Osm/L to about 1,000 Osm/L, about 300 Osm/L to about 900 Osm/L, about 300 Osm/L to about 800 Osm/L, about 300 Osm/L to about 700 Osm/L, about 300 Osm/L to about 600 Osm/L, about 300 Osm/L to about 500 Osm/L, about 300 Osm/L to about 400 Osm/L, about 400 Osm/L to about 3,500 Osm/L, about 400 Osm/L to about 3,000 Osm/L, about 400 Osm/L to about 2,500 Osm/L, about 400 Osm/L to about 2,500 Osm/L, about 400 Osm/L to about 2,000 Osm/L, about 400 Osm/L to about 2,000 Osm/L, about 400 Osm/L to about 1,500 Osm/L, about 400 Osm/L to about 1,000 Osm/L, about 400 Osm/L to about 900 Osm/L, about 400 Osm/L to about 800 Osm/L, about 400 Osm/L to about 700 Osm/L, about 400 Osm/L to about 6010 Osm/L, about 400 Osm/L to about 500 Osm/L, about 500 Osm/L to about 3,500 Osm/L, about 500 Osm/L to about 3,000 Osm/L, about 500 Osm/L to about 2,500 Osm/L, about 500 Osm/L to about 2,000 Osm/L, about 500 Osm/L to about 1,500 Osm/L, about 500 Osm/L to about 1,000 Osm/L, about 500 Osm/L to about 900 Osm/L, about 500 Osm/L to about 800 Osm/L, about 500 Osm/L to about 700 Osm/L, about 500 Osm/L to about 600 Osm/L, about 600 Osm/L to about 3,500 Osm/L, about 600 Osm/L to about 3,000 Osm/L, about 600 Osm/L to about 2,500 Osm/L, about 600 Osm/L to about 2,000 Osm/L, about 600 Osm/L to about 1,500 Osm/L, about 600 Osm/L to about 1,000 Osm/L, about 600 Osm/L to about 900 Osm/L, about 600 Osm/L to about 800 Osm/L, about 600 Osm/L to about 700 Osm/L, about 700 Osm/L, to about 3,500 Osm/L, about 700 Osm/L to about 3,000 Osm/L, about 700 Osm/L to about 2,500 Osm/L, about 700 Osm/L to about 2,000 Osm/L, about 700 Osm/L to about 1,500 Osm/L, about 700 Osm/L to about 1,000 Osm/L, about 700 Osm/L to about 900 Osm/L, about 700 Osm/L to about 800 Osm/L, about 800 Osm/L to about 3,500 Osm/L, about 800 Osm/L to about 3,000 Osm/L, about 800 Osm/L, to about 2,500 Osm/L, about 800 Osm/L to about 2,000 Osm/L, about 800 Osm/L to about 1,500 Osm/L, about 800 Osm/L to about 1,000 Osm/L, about 800 Osm/L to about 900 Osm/L, about 900 Osm/L to about 3,500 Osm/L, about 900 Osm/L to about 3,000 Osm/L, about 900 Osm/L to about 2,500 Osm/L, about 900 Osm/L to about 2,000 Osm/L, about 900 Osm/L to about 1,500 Osm/L, about 900 Osm/L to about 1,000 Osm/L, about 1,000 Osm/L to about 3,500 Osm/L, about 1,000 Osm/L to about 3,000 Osm/L, about 1,000 Osm/L to about 2,500 Osm/L, about 1,000 Osm/L to about 2,000 Osm/L, about 1,000 Osm/L to about 1,500 Osm/L, about 1,500 Osm/L to about 3,500 Osm/L, about 1,500 Osm/L to about 3,000 Osm/L, about 1,500 Osm/L to about 2,500 Osm/L, about 1,500 Osm/L to about 2,000 Osm/L, about 2,000 Osm/L to about 3,500 Osm/L, about 2,000 Osm/L to about 3,000 Osm/L, about 2,000 Osm/L to about 2,500 Osm/L, about 2,500 Osm/L to about 3,500 Osm/L, about 2,500 Osm/L to about 3,000 Osm/L, or about 3,000 Osm/L to about 3,500 Osm/L, when solvated in an aqueous solvent.

In certain embodiments, the composition has an osmolality in the range of about 300 Osm/L to about 3,000 Osm/L, about 400 Osm/L to about 3,000 Osm/L, or about 500 Osm/L to about 3,000 Osm/L, when solvated in an aqueous solvent.

In certain embodiments, the composition has an osmolality of about 300 Osm/L, about 350 Osm/L, about 400 Osm/L, about 450 Osm/L, about 500 Osm/L, about 600 Osm/L, about 700 Osm/L, about 800 Osm/L, about 900 Osm/L, about 1,000 Osm/L, about 1,250 Osm/L, about 1,500 Osm/L, about 1,750 Osm/L, about 2,000 Osm/L, about 2,250 Osm/L, about 2,500 Osm/L, about 2,750 Osm/L, about 3,000 Osm/L, about 3,500 Osm/L, or about 4,000 Osm/L, when solvated in an aqueous solvent.

The osmolality can be modulated using a variety of agents such as salts, saccharides and polymers. Exemplary salts include, without limitation, NaCl, KCl, MgCl₂, CaCl₂), Na₂HPO₄, NaH₂PO₄, K₂HPO₄, KH₂PO₄, or combinations thereof. Exemplary saccharides include, without limitation, monosaccharides and disaccharides, e.g., glucose, sucrose, trehalose. Exemplary polymers include, without limitation, alginate, acacia, alginic acid, an agar, alamic acid, bentonite, carbomer, a carrageenan, a cellulose, a dextrin, a gelatin, gellan gum, guar gum, carboxymethylcellulose, hydroxyethylcellulose, hydroxypropylcellulose, methylcellulose, mucin, pectin, starch, xanthan gum, or a combination thereof.

pH

The pH is selected to maintain the viability of the microorganism or microorganisms being preserved. In certain embodiments, the composition has a pH in the range of about 4 to about 8, about 4.5 to about 8, about 5 to about 8, about 5.5 to about 8, about 6 to about 8, about 6.5 to about 8, about 7 to about 8, about 7.5 to about 8, about 4 to about 7.5, about 4 to about 7, about 4 to about 6.5, about 4 to about 6, about 4 to about 5.5, about 4 to about 5, about 4 to about 4.5, about 4.5 to about 7.5, about 4.5 to about 7, about 4.5 to about 6.5, about 4.5 to about 6, about 4.5 to about 5.5, about 4.5 to about 5, about 5 to about 7.5, about 5 to about 7, about 5 to about 6.5, about 5 to about 6, about 5 to about 5.5, about 5.5 to about 7.5, about 5.5 to about 7, about 5.5 to about 6.5, about 5.5 to about 6, about 6 to about 7.5, about 6 to about 7, about 6 to about 6.5, about 6.5 to about 7.5, about 6.5 to about 7, or about 7 to about 7.5, when solvated in an aqueous solvent.

In certain embodiments, the composition has a pH in the range of about 4 to about 8, about 5 to about 8, about 4 to about 7.5, about 5 to about 7.5, or about 5.5 to about 7.5, when solvated in an aqueous solvent.

In certain embodiments, the composition has a pH of about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, or about 8, when solvated in an aqueous solvent.

### Energy Source for the Organisms

In certain embodiments, the energy source is a cell culture media that permits growth of the microorganisms. It is understood that the culture media may vary depending upon the anaerobic microorganisms of interest, and so may be tailored to a given microorganism or group of microorganisms. Exemplary culture media may contain one or more of the following components, including, without limitation, a carbon source (e.g., a sugar), a nitrogen source (e.g., ammonia rich components (e.g., yeast extract, peptones) and amino acids), vitamins, minerals (e.g., phosphate, acetate), buffering components (e.g., sodium phosphate or potassium phosphate buffers), trace metals, fatty acids, and oxygen scavengers (e.g., cysteine, sodium dithionite, sodium sulfite, thioglycolate, iron sulfide, dithiothreitol).

In certain embodiments, the composition comprises about 3% (w/w) to about 15% (w/w), about 5% (w/w) to about 15% (w/w), about 7% (w/w) to about 15% (w/w), about 9% (w/w) to about 15% (w/w), about 11% (w/w) to about 15% (w/w), about 13% (w/w) to about 15% (w/w), about 3% (w/w) to about 13% (w/w), about 3% (w/w) to about 11% (w/w), about 30% (w/w) to about 9% (w/w), about 3% (w/w) to about 7% (w/w), about 3% (w/w) to about 5% (w/w), about 5% (w/w) to about 13% (w/w), about 5% (w/w) to about 11% (w/w), about 5% (w/w) to about 9% (w/w), about 5% (w/w) to about 7% (w/w), about 7% (w/w) to about 13% (w/w), about 7% (w/w) to about 11% (w/w), about 7% (w/w) to about 90% (w/w), about 9% (w/w) to about 13% (w/w), about 9% (w/w) to about 11% (w/w), or about 11% (w/w) to about 13% (w/w), of the energy source, when solvated in an aqueous solvent. In certain embodiments, the composition comprises about 3% (w/w) to about 15% (w/w) of the energy source, when solvated in an aqueous solvent.

In certain embodiments, the composition comprises about 3% (w/w), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), or about 15% (w/w) of the energy source.

In certain embodiments, the energy source is a monosaccharide, a disaccharide, an oligosaccharide, or a combination thereof. In certain embodiments, the monosaccharide is selected from the group consisting of glucose, fructose, xylose, and combinations thereof. In certain embodiments, the disaccharide is trehalose, sucrose, or a combination thereof. In certain embodiments, the oligosaccharide is an inulin derivative, e.g. a fructo-oligosaccharide, e.g., raftilose. In certain embodiments, the oligosaccharide is maltodextrin.

In certain embodiments, the energy source comprises raftilose and maltodextrin.

In certain embodiments, the composition comprises about 0.5% (w/w) to about 5% (w/w), about 1% (w/w) to about 5% (w/w), about 1.5% (w/w) to about 5% (w/w), about 2% (w/w) to about 5% (w/w), about 2.5% (w/w) to about 5% (w/w), about 3% (w/w) to about 5% (w/w), about 3.5% (w/w) to about 5% (w/w), about 4% (w/w) to about 5% (w/w), about 4.5% (w/w) to about 5% (w/w), about 0.5% (w/w) to about 4.5% (w/w), about 0.5% (w/w) to about 4% (w/w), about 0.5% (w/w) to about 3.5% (w/w), about 0.5% (w/w) to about 3% (w/w), about 0.5% (w/w) to about 2.5% (w/w), about 0.5% (w/w) to about 2% (w/w), about 0.5% (w/w) to about 1.5% (w/w), about 0.5% (w/w) to about 1% (w/w), about 1% (w/w) to about 4.5% (w/w), about 1% (w/w) to about 4% (w/w), about 1% (w/w) to about 3.5% (w/w), about 1% (w/w) to about 3% (w/w), about 1% (w/w) to about 2.5% (w/w), about 1% (w/w) to about 2% (w/w), about 1% (w/w) to about 1.5% (w/w), about 1.5% (w/w) to about 4.5% (w/w), about 1.5% (w/w) to about 4% (w/w), about 1.5% (w/w) to about 3.5% (w/w), about 1.5% (w/w) to about 3% (w/w), about 1.5% (w/w) to about 2.5% (w/w), about 1.5% (w/w) to about 2% (w/w), about 2% (w/w) to about 4.5% (w/w), about 2% (w/w) to about 4% (w/w), about 2% (w/w) to about 3.5% (w/w), about 2% (w/w) to about 3% (w/w), about 2% (w/w) to about 2.5% (w/w), about 2.5% (w/w) to about 4.5% (w/w), about 2.5% (w/w) to about 4% (w/w), about 2.5% (w/w) to about 3.5% (w/w), about 2.5% (w/w) to about 3% (w/w), about 3% (w/w) to about 4.5% (w/w), about 3% (w/w) to about 4% (w/w), about 3% (w/w) to about 3.5% (w/w), about 3.5% (w/w) to about 4.5% (w/w), about 3.5% (w/w) to about 4% (w/w), or about 4% (w/w) to about 4.5% (w/w) raftilose, when solvated in an aqueous solvent. In certain embodiments, the composition comprises about 0.5% (w/w) to about 5% (w/w) raftilose, when solvated in an aqueous solvent.

In certain embodiments, the composition comprises about 0.5% (w/w), about 1% (w/w), about 1.5% (w/w), about 2% (w/w), about 3% (w/w), about 3.5% (w/w), about 4% (w/w), about 4.5% (w/w), or about 5% (w/w) raftilose, when solvated in an aqueous solvent.

In certain embodiments, the composition comprises about 3% (w/w) to about 10% (w/w), about 4% (w/w) to about 10% (w/w), about 5% (w/w) to about 10% (w/w), about 6% (w/w) to about 10% (w/w), about 7% (w/w) to about 10% (w/w), about 8% (w/w) to about 10% (w/w), about 9% (w/w) to about 10% (w/w), about 3% (w/w) to about 9% (w/w), about 3% (w/w) to about 8% (w/w), about 3% (w/w) to about 7% (w/w), about 3% (w/w) to about 6% (w/w), about 3% (w/w) to about 5% (w/w), about 3% (w/w) to about 4% (w/w), about 4% (w/w) to about 9% (w/w), about 4% (w/w) to about 8% (w/w), about 4% (w/w) to about 7% (w/w), about 4% (w/w) to about 6% (w/w), about 4% (w/w) to about 5% (w/w), about 5% (w/w) to about 9% (w/w), about 5% (w/w) to about 8% (w/w), about 5% (w/w) to about 7% (w/w), about 5% (w/w) to about 6% (w/w), about 6% (w/w) to about 9% (w/w), about 6% (w/w) to about 8% (w/w), about 6% (w/w) to about 7% (w/w), about 7% (w/w) to about 9% (w/w), about 7% (w/w) to about 8% (w/w), or about 8% (w/w) to about 9% (w/w) maltodextrin, when solvated in an aqueous solvent. In certain embodiments, the composition comprises about 3% (w/w) to about 10% (w/w) maltodextrin, when solvated in an aqueous solvent.

In certain embodiments, the composition comprises about 3% (w/w), about 3.5% (w/w), about 4% (w/w), about 4.5% (w/w), about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) maltodextrin, when solvated in an aqueous solvent.

### Redox Potential

It is understood that the cell preservative (e.g., cryopreservative) media will have a redox potential that facilitates the viability, and growth and/or replication of the microorganisms of interest. The redox potential can be tailored to meet the preferred conditions of a given microorganism or a group of microorganisms.

For anaerobic bacteria (e.g., the microorganisms described herein) it is required that a composition (e.g., a preservative or cryopreservative composition described herein) be in a reduced state (i.e., a negative redox potential) so that the organisms can be maintained in their natural metabolic state and maintain viability.

The preparation of a preservative composition described herein with the desired negative redox potential may be achieved using one or more of the following approaches:
(1) The addition of pre-reduced anaerobic media to the anaerobic microorganisms prior to their growth and/or harvesting, or after harvesting. The anaerobic media may contain reducing agents (e.g., cysteine, sodium dithionite, sodium sulfite, thioglycolate, iron sulfide, dithiothreitol) and redox mediators (e.g., heavy metals (e.g., iron, zinc, manganese, cobalt, copper, nickel, and molybdenum) and vitamins (e.g., menadione, ascorbic acid, folic acid, riboflavin, pyridoxine, biotin, thiamine, nicotinamide, D-pathothenic acid, vitamin b12, lipoic acid, and 4-aminobenzoic acid)) that are can assist in maintaining a reduced environment.
(2) Removing oxygen from the reagents and materials (e.g., growth media and other components) used to make, store and use the compositions described herein. Such techniques may include, but are not limited to, bubbling an anaerobic inert gas (e.g., nitrogen, carbon dioxide, argon, etc.) through liquid compositions or component blends (see Example 2 disclosed herein).
(3) Adding reducing agents (e.g., cysteine, sodium dithionite, sodium sulfite, thioglycolate, iron sulfide, dithiothreitol) and/or redox mediators (e.g., heavy metals (e.g., iron, zinc, manganese, cobalt, copper, nickel, and molybdenum) and vitamins (e.g., menadione, ascorbic acid, folic acid, riboflavin, pyridoxine, biotin, thiamine, nicotinamide, D-pathothenic acid, vitamin b12, lipoic acid, and 4-aminobenzoic acid)) to the compositions described herein to maintain anaerobicity.
(4) Performing the various process steps (e.g. (i) blending the anaerobic microorganisms with the media, cryopreservatives, etc., (ii) pouring the resulting compositions into lyophilization trays, and (iii) freezing the compositions) in an anaerobic environment.

In certain embodiments, the redox potential will be in the range of +300 to −100 mV for facultative and +100 mV to about −500 mV for obligate anaerobes. The redox potential is typically measured by a sensing electrode (e.g., a standard hydrogen electrode, a silver chloride electrode, or a saturated calomel electrode. Exemplary redox potential measuring instruments include an Metler Toledo ORP meter and probe.

In certain embodiments, the composition has a redox potential in the range of −50 mV to 500 mV, −50 mV to −450 mV, −50 mV to −400 mV, −50 mV to −350 mV, −50 mV to −300 mV, −50 mV to −250 mV, −50 mV to −200 mV, −50 mV to −150 mV, −50 mV to −100 mV, −100 mV to −500 mV, −100 mV to −450 mV, −100 mV to −400 mV, −100 mV to −350 mV, −100 mV to −300 mV, 100 mV to −250 mV, −100 mV to −200 mV, −100 mV to −150 mV, −150 mV to −500 mV, −150 mV to −450 mV, −150 mV to −400 mV, −150 mV to −350 mV, −150 mV to −300 mV, −150 mV to 250 mV, −150 mV to −200 mV, −200 mV to −500 mV, −200 mV to −450 mV, −200 mV to −400 mV, −200 mV to −350 mV, −200 mV to −300 mV, −200 mV to −250 mV, −250 mV to −500 mV, 250 mV to −450 mV, −250 mV to −400 mV, −250 mV to −350 mV, −250 mV to −300 mV, −300 mV to −500 mV, −300 mV to −450 mV, −300 mV to −400 mV, −300 mV to −350 mV, −350 mV to 500 mV, −350 mV to −450 mV, −350 mV to −400 mV, −400 mV to −500 mV, −400 mV to −450 mV, or −450 mV to −500 mV, for example, when solvated in an aqueous solvent. In certain embodiments, the composition has a redox potential in the range of −50 mV to −500 mV, when solvated in an aqueous solvent.

In certain embodiments, the composition has a redox potential of about −50 mV, about 60 mV, about −70 mV, about −80 mV, about −90 mV, about −100 mV, about −110 mV, about −120 mV, about −130 mV, about −140 mV, about −150 mV, about −160 mV, about −170 mV, about 180 mV, about −190 mV, about −200 mV, about −240 mV, about −260 mV, about −280 mV, about −300 mV, about −320 mV, about −340 mV, about −360 mV, about −380 mV, about −400 mV, about −420 mV, about −440 mV, about −460 mV, about −480 mV, or about −500 mV, for example, when solvated in an aqueous solvent.

### Microorganisms

In certain embodiments, the composition further comprises one or more anaerobic microorganisms that are to be preserved. The anaerobic microorganisms can be obligate anaerobes or facultative anaerobes.

Exemplary anaerobic microorganism include one of more of Bifidobacteriaceae, Coriobacteriaceae, Corynebacteriaceae, Dietziaceae, Mycobacteriaccae, Gordoniaceae, Nocardiaceae, Streptomycetaceae, Nocardiaceae 2, Propionibacterineae, Micrococcaceae, Brevibacteriaceae, Dermabacteraceae, Actinomycetaceae, Bogoriellaceae, Promicromonosporaceae, Microbacteriaceae, Cellulomonadaceae, Dermacoccaceae, Intrasporangiaceae, Geodermatophilaceae, Micromonosporaceae, Bacteroidaceae, Prevotellaceae, Porphyromonadaceae 1, Rikenellaceae, Porphyromonadaceae 2, Flavobacteriaceae, Sphingobacteriaceae, Unclassified Bacteroidetes. Cytophagaceae, Epsilonproteobacteria, Firmicutes, Lactobacillaccae, Leuconostocaceae, Aerococcaceae, Carnobacteriaceae, Aerococcaceae 2, Lachnospiraceae, Streptococcaceae, Enterococcaceae, Erysipelotrichaceae, Staphylococcaceae, Family XI Incertae Sedis, Listeriaceae, Planococcaceae 1, Family XII Incertae Sedis, Planococcaceae 2, Bacillaceae, Paenibacillaceae, Clostridiaceae (cluster I), Clostridiaceae (cluster II), Lachnospiraceae (cluster XIVa), Ruminococcaceae (cluster IV), Peptostreptococcaceae (cluster XI), Erysipelotrichaceae (clusters XVII & XVIII), Fusobacteria, Ruminococcaceae, Unclassified Clostridiales, Oscillospiraceae, Lachnospiraceae, Eubacteriaceae, Family XIII Incertae Sedis, Peptostreplococcaceae (cluster XI), Lachnospiraceae (cluster XIVa), Ruminococcaceae (cluster IV), Clostridiaceae (cluster I), Erysipelotrichaceae (clusters XVII & XVIII), Peptostreptococcaceae, Family XI Incertae Sedis, Erysipelotrichi and Bacilli, Clostridia, Negativicutes, Clostridia 2, Clostridia 3/Halanaerobiales, Veillonellaceae, Acidaminococcaceae, Erysipelotrichaceae cluster XVII, Erysipelotrichaceae cluster XVIII, Erysipelotrichaceae cluster XVI, Tenericutes, Erysipelotrichaceae, Fusobacteriaceae, Leptotrichiaceae, Rhodobacteraceae, Brucellaceae, Rhizobiaceae, Phyllobacteriaceae, Aurantimonadaceae, Bradyrhizobiaceae, Methylobacteriaceae, Xanthobacteraceae, Caulobacteraceae, Hyphomicrobiaceae, Sphingomonadaceae, Acetobacteraceae, Rhodospirillaceae, Firmicutes, Comamonadaceae, Sutterellaceae, Alcaligenaceae, Burkholderiaceae 1, Oxalobacteraceae, Burkholderiaceae 2, Rhodocyclaceae, Neisseriaceae, Enterobacteriaceae, Pasteurellaceae, Vibrionaceae, Aeromonadaceae, Succinivibrionaceae, Moraxellaceae, Pseudomonadaceae, Xanthomonadaceae, Sinobacteraceae, ε-Campylobacteraceae 1, ε-Helicobacteraceae, ε-Campylobacteraceae 2, δDesulfovibrionaceae, Verrucomicrobia, Lentisphaerae, Planctomycetes, Spirochaetes, Deinococcus-Thermus, Synergistetes, Archaea/Euryarchaeota, Archaea/Crenarchaeota, Fungi Ascomycota, Fungi-Basidiomycota, Stramenopiles, Apicomplexa, Amoebozoa, Ciliophora, Metamonada, Microsporidia, Fornicata, Akkermansiaceae, Christensenellaceae, Odoribacteraceae, Eggerthellaceae, and combinations thereof.

In certain embodiments, the one or more anaerobic microorganisms is selected from the group consisting of Akkermansiaceae, Bacillaceae, Bacteroidaceae, Bifidobacteriaceae, Christensenellaceae, Clostridiaceae, Coriobacteriaceae, Eggerthellaceae, Enterobacteriaceae, Eubacteriaceae, Lachnospiraceae, Lactobacillaceae, Leuconostocaceae, Odoribacteraceae, Oxalobacteraceae, Porphyromonadaceae, Prevotellaceae, Rikenellaceae, Ruminococcaceae, Streptococcaceae, and combinations thereof.

The one or more anaerobic microorganisms can be present in the compositions described herein in the form of a cell paste or cell slurry. In certain embodiments, the composition comprises about 30% (w/w) to about 50% (w/w), about 32% (w/w) to about 50% (why), about 34% (w/w) to about 50% (w/w), about 36% (w/w) to about 50% (why), about 38% (w/w) to about 50% (w/w), about 40% (w/w) to about 50% (w/w), about 42% (w/w) to about 50% (w/w), about 44% (w/w) to about 50% (w/w), about 46% (w/w) to about 50% (w/w), about 48% (w/w) to about 50% (w/w), about 30% (w/w) to about 48% (w/w), about 30% (w/w) to about 46% (w/w), about 30% (w/w) to about 44% (w/w), about 30% (w/w) to about 42% (w/w), about 30% (w/w) to about 40% (w/w), about 30% (w/w) to about 38% (w/w), about 30% (why) to about 36% (w/w), about 30% (w/w) to about 34% (w/w), about 30% (w/w) to about 32% (w/w), about 32% (w/w) to about 48% (w/w), about 32% (w/w) to about 46% (why), about 32% (w/w) to about 44% (w/w), about 32% (w/w) to about 42% (w/w), about 32% (w/w) to about 40% (w/w), about 32% (w/w) to about 38% (w/w), about 32% (w/w) to about 36% (w/w), about 32% (w/w) to about 34% (w/w), about 34% (w/w) to about 48% (w/w), about 34% (w/w) to about 46% (w/w), about 34% (w/w) to about 44% (w/w), about 34% (w/w) to about 42% (why), about 34% (w/w) to about 40% (w/w), about 34% (why) to about 38% (w/w), about 34% (why) to about 36% (w/w), about 36% (w/w) to about 48% (w/w), about 36% (w/w) to about 46% (w/w), about 36% (w/w) to about 44% (w/w), about 36% (w/w) to about 42% (why), about 36% (w/w) to about 40% (w/w), about 36% (w/w) to about 38% (w/w), about 38% (w/w) to about 48% (w/w), about 38% (w/w) to about 46% (w/w), about 38% (w/w) to about 44% (w/w), about 38% (w/w) to about 42% (w/w), about 38% (w/w) to about 40% (w/w), about 40% (w/w) to about 48% (w/w), about 40% (w/w) to about 46% (w/w), about 40% (w/w) to about 44% (w/w), about 40% (w/w) to about 42% (w/w), about 42% (w/w) to about 48% (w/w), about 42% (w/w) to about 46% (w/w), about 42% (w/w) to about 44% (w/w), about 44% (w/w) to about 48% (w/w), about 44% (w/w) to about 46% (w/w), or about 46% (w/w) to about 48% (w/w), of the cell paste or cell slurry, when solvated in an aqueous solvent. In certain embodiments, the composition comprises about 30% (w/w) to about 50% (w/w) of the cell paste or cell slurry, when solvated in an aqueous solvent.

In certain embodiments, the composition comprises about 30% (w/w), about 31% (w/w), about 32% (w/w), about 33% (w/w), about 34% (w/w), about 35% (w/w), about 36% (w/w), about 37% (w/w), about 38% (w/w), about 39% (w/w), about 40% (w/w), about 41% (w/w), about 42% (w/w), about 43% (w/w), about 44% (w/w), about 45% (w/w), about 46% (w/w), about 47% (w/w), about 48% (w/w), about 49% (w/w), or about 50% (w/w) of the cell paste or cell slurry, when solvated in an aqueous solvent.

In certain embodiments, the one or more anaerobic microorganisms are present in the form of a cell paste, e.g., compact solids. In certain embodiments, the one or more anaerobic microorganisms are present in the form of a cell slurry, e.g. a cell suspension.

### Stabilizers

In certain embodiments, the composition can further comprise a protein stabilizer. In certain embodiments, the composition comprises about 5% (w/w) to about 10% (w/w), about 6% (w/w) to about 10% (w/w), about 7% (w/w) to about 10% (w/w), about 8% (w/w) to about 10% (w/w), about 9% (w/w) to about 10% (w/w), about 5% (w/w) to about 9% (w/w), about 5% (w/w) to about 8% (w/w), about 5% (w/w) to about 7% (w/w), about 5% (w/w) to about 6% (w/w), about 6% (w/w) to about 9% (w/w), about 6% (w/w) to about 8% (w/w), about 6% (w/w) to about 7% (w/w), about 7% (w/w) to about 9% (w/w), about 7% (w/w) to about 8% (w/w), or about 8% (w/w) to about 9% (w/w) of the protein stabilizer, when solvated in an aqueous solvent.

In certain embodiments, the composition comprises about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) of the protein stabilizer, when solvated in an aqueous solvent.

In certain embodiments, the protein stabilizer is selected from the group consisting of an amino acid, an antioxidant, a disaccharide, a reducing agent, polyethylene glycol, and combinations thereof. In certain embodiments, the disaccharide is selected from the group consisting of trehalose, sucrose, isomaltose, lactose, maltose, lactulose, cellobiose, and combinations thereof. In certain embodiments, the protein stabilizer comprises trehalose. In certain embodiments, the protein stabilizer comprises sucrose and trehalose.

In certain embodiments, the composition comprises about 5% (w/w) to about 10% (w/w), about 6% (w/w) to about 10% (w/w), about 7% (w/w) to about 10% (w/w), about 8% (w/w) to about 10% (w/w), about 9% (w/w) to about 10% (w/w), about 5% (w/w) to about 9% (w/w), about 5% (w/w) to about 8% (w/w), about 5% (w/w) to about 7% (w/w), about 5% (w/w) to about 6% (w/w), about 6% (w/w) to about 9% (w/w), about 6% (w/w) to about 8% (w/w), about 6% (w/w) to about 7% (w/w), about 7% (w/w) to about 9% (w/w), about 7% (w/w) to about 8% (w/w), or about 8% (w/w) to about 9% (w/w) trehalose, when solvated in an aqueous solvent. In certain embodiments, the composition comprises about 5% (w/w) to about 10% (w/w) trehalose, when solvated in an aqueous solvent.

In certain embodiments, the composition comprises about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) trehalose, when solvated in an aqueous solvent.

In certain embodiments, the composition comprises 0% (w/w) to about 10% (w/w), about 1% (w/w) to about 10% (w/w), about 2% (w/w) to about 10% (w/w), about 3% (w/w) to about 10% (w/w), about 4% (w/w) to about 10% (w/w), about 5% (w/w) to about 10% (w/w), about 6% (w/w) to about 10% (w/w), about 7% (w/w) to about 10% (w/w), about 8% (w/w) to about 10% (w/w), about 9% (w/w) to about 10% (w/w), 0% (w/w) to about 9% (w/w), 0% (w/w) to about 8% (w/w), 0% (w/w) to about 7% (w/w), 0% (w/w) to about 6% (w/w), 0% (w/w) to about 5% (w/w), 0% (w/w) to about 4% (w/w), 0% (w/w) to about 3% (w/w), 0% (w/w) to about 2% (w/w), 0% (w/w) to about 1% (w/w), about 1% (w/w) to about 9% (w/w), about 1% (w/w) to about 8% (w/w), about 1% (w/w) to about 7% (w/w), about 1% (w/w) to about 6% (w/w), about 1% (w/w) to about 5% (w/w), about 1% (w/w) to about 4% (w/w), about 1% (w/w) to about 3% (w/w), about 1% (w/w) to about 2% (w/w), about 2% (w/w) to about 9% (w/w), about 2% (w/w) to about 8% (w/w), about 2% (w/w) to about 7% (w/w), about 2% (w/w) to about 6% (w/w), about 2% (w/w) to about 5% (w/w), about 2% (w/w) to about 4% (w/w), about 2% (w/w) to about 3% (w/w), about 3% (w/w) to about 9% (w/w), about 3% (w/w) to about 8% (w/w), about 3% (w/w) to about 7% (w/w), about 3% (w/w) to about 6% (w/w), about 3% (w/w) to about 5% (w/w), about 3% (w/w) to about 4% (w/w), about 4% (w/w) to about 9% (w/w), about 4% (w/w) to about 8% (w/w), about 4% (w/w) to about 7% (w/w), about 4% (w/w) to about 6% (w/w), about 4% (w/w) to about 5% (w/w), about 5% (w/w) to about 9% (w/w), about 5% (w/w) to about 8% (w/w), about 5% (w/w) to about 7% (w/w), about 5% (w/w) to about 6% (w/w), about 6% (w/w) to about 9% (w/w), about 6% (w/w) to about 8% (w/w), about 6% (w/w) to about 7% (w/w), about 7% (w/w) to about 9% (w/w), about 7% (w/w) to about 8% (w/w), or about 8% (w/w) to about 9% (w/w) sucrose, when solvated in an aqueous solvent. In certain embodiments, the composition comprises about 5% (w/w) to about 10% (w/w) sucrose, when solvated in an aqueous solvent. In certain embodiments, the composition comprises 0% (w/w) to about 5% (w/w) sucrose, when solvated in an aqueous solvent.

In certain embodiments, the composition comprises about 0.5% (w/w), about 1% (w/w), about 1.5% (w/w), about 2% (w/w), about 2.5% (w/w), about 3% (w/w), about 3.5% (w/w), about 4% (w/w), about 4.5% (w/w), about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) sucrose, when solvated in an aqueous solvent.

### Solvated Compositions

In certain embodiments, when the composition is solvated in an aqueous solvent, e.g., water or a water-based solvent, the composition comprises about 30% (w/w) to about 60% (w/w), about 35% (w/w) to about 60% (w/w), about 40% (w/w) to about 60% (w/w), about 45% (w/w) to about 60% (w/w), about 50% (w/w) to about 60% (w/w), about 55% (w/w) to about 60% (w/w), about 30% (w/w) to about 55% (w/w), about 30% (w/w) to about 50% (w/w), about 30% (w/w) to about 45% (w/w), about 30% (w/w) to about 40% (w/w), about 30% (w/w) to about 35% (w/w), about 35% (w/w) to about 55% (w/w), about 35% (w/w) to about 50% (w/w), about 35% (w/w) to about 45% (w/w), about 35% (w/w) to about 40% (w/w), about 40% (w/w) to about 55% (w/w), about 40% (w/w) to about 50% (w/w), about 40% (w/w) to about 45% (w/w), about 45% (w/w) to about 55% (w/w), about 45% (w/w) to about 50% (w/w), or about 50% (w/w) to about 55% (w/w) of the aqueous solvent.

In certain embodiments, when the composition is solvated in such an aqueous solvent, the composition comprises about 30% (w/w), about 31% (w/w), about 32% (w/w), about 33% (w/w), about 34% (w/w), about 35% (w/w), about 36% (w/w), about 37% (w/w), about 38% (w/w), about 39% (w/w), about 40% (w/w), about 41% (w/w), about 42% (w/w), about 43% (w/w), about 44% (w/w), about 45% (w/w), about 46% (w/w), about 47% (w/w), about 48% (w/w), about 49% (w/w), about 50% (w/w), about 51% (w/w), about 52% (w/w), about 53% (w/w), about 54% (w/w), about 55% (w/w), about 56% (w/w), about 57% (w/w), about 58% (w/w), about 59% (w/w), or about 60% (w/w) of the aqueous solvent.

In certain embodiments, the aqueous solvent is water (e.g. ionized water or deionized water). In certain embodiments, when the composition is solvated in water, the composition comprises about 30% (w/w) to about 60% (w/w), about 35% (w/w) to about 60% (w/w), about 40% (w/w) to about 60% (w/w), about 45% (w/w) to about 60% (w/w), about 50% (w/w) to about 60% (w/w), about 55% (w/w) to about 60% (w/w), about 30% (w/w) to about 55% (w/w), about 30% (w/w) to about 50% (w/w), about 30% (w/w) to about 45% (w/w), about 30% (w/w) to about 40% (w/w), about 30% (w/w) to about 35% (w/w), about 35% (w/w) to about 55% (w/w), about 35% (w/w) to about 50% (w/w), about 35% (w/w) to about 45% (w/w), about 35% (w/w) to about 40% (w/w), about 40% (w/w) to about 55% (w/w), about 40% (w/w) to about 50% (w/w), about 40% (w/w) to about 45% (w/w), about 45% (w/w) to about 55% (w/w), about 45% (w/w) to about 50% (w/w), or about 50% (w/w) to about 55% (w/w) water.

In certain embodiments, when the composition is solvated in water, the composition comprises about 30% (w/w), about 31% (w/w), about 32% (w/w), about 33% (w/w), about 34% (w/w), about 35% (w/w), about 36% (w/w), about 37% (w/w), about 38% (w/w), about 39% (w/w), about 40% (w/w), about 41% (w/w), about 42% (w/w), about 43% (w/w), about 44% (w/w), about 45% (w/w), about 46% (w/w), about 47% (w/w), about 48% (w/w), about 49% (w/w), about 50% (w/w), about 51% (w/w), about 52% (w/w), about 53% (w/w), about 54% (w/w), about 55% (w/w), about 56% (w/w), about 57% (w/w), about 58% (w/w), about 59% (w/w), or about 60% (w/w) water.

### Exemplary Preservative Compositions

In various embodiments, provided herein is a composition for preserving anaerobic microorganisms, which comprises, among other things, a viscosity modifier, an energy source for the microorganisms, and an aqueous solvent.

In various embodiments, the composition for preserving anaerobic microorganisms comprises:
(i) about 3% (w/w) to about 15% (w/w) of an energy source for the microorganisms;
(ii) about 0.01% (w/w) to about 0.4% (w/w) of a viscosity modifier; and
(iii) about 30% (w/w) to about 60% (w/w) of an aqueous solvent.

In various embodiments, the composition comprises a viscosity modifier, an energy source for the microorganisms, and an aqueous solvent, wherein the composition has a viscosity in the range of about 5 cP to about 6,000 cP, an osmolality in the range of about 300 Osm/L to about 4,000 Osm/L, a pH in the range of about 4 to about 8, and a redox potential in the range of from about −50 mV to about −500 mV.

In various embodiments, the composition for preserving anaerobic microorganisms comprises:
(i) about 3% (w/w) to about 15% (w/w) of an energy source for the microorganisms;
(ii) about 0.01% (w/w) to about 0.4% (w/w) of a viscosity modifier; and
(iii) about 30% (w/w) to about 60% (w/w) of an aqueous solvent, wherein the composition has a viscosity in the range of about 5 cP to about 6,000 cP, an osmolality in the range of about 300 Osm/L to about 4,000 Osm/L, a pH in the range of about 4 to about 8, and a redox potential in the range of from about −50 mV to about −500 mV.

In various embodiments, the composition comprises:
(i) about 30% (w/w) to about 50% (w/w) of a cell paste, wherein the cell paste comprises one or more anaerobic microorganisms;
(ii) about 3% (w/w) to about 15% (w/w) of an energy source for the microorganisms;
(iii) about 0.01% (w/w) to about 0.4% (w/w) of a viscosity modifier; and
(iv) about 30% (w/w) to about 60% (w/w) of an aqueous solvent.

In various embodiments, the composition comprises:
(i) about 30% (w/w) to about 50% (w/w) of a cell paste, wherein the cell paste comprises one or more anaerobic microorganisms;
(ii) about 3% (w/w) to about 15% (w/w) of an energy source for the microorganisms;
(iii) about 0.01% (w/w) to about 0.4% (w/w) of a viscosity modifier; and
(iv) about 30% (w/w) to about 60% (w/w) of an aqueous solvent, wherein the composition has a viscosity in the range of about 5 cP to about 6,000 cP, an osmolality in the range of about 300 Osm/L to about 4,000 Osm/L, a pH in the range of about 4 to about 8, and a redox potential in the range of from about −50 mV to about −500 mV.

In various embodiments, the composition comprises:
(i) about 30% (w/w) to about 50% (w/w) of a cell paste, wherein the cell paste comprises one or more anaerobic microorganisms;
(ii) about 0.5% (w/w) to about 5% (w/w) raftilose;
(iii) about 3% (w/w) to about 10% (w/w) maltodextrin;
(iv) about 0% (w/w) to about 0.4% (w/w) sodium alginate;
(v) about 5% (w/w) to about 10% (w/w) trehalose;
(vi) 0% (w/w) to about 5% (w/w) sucrose; and
(vii) about 35% (w/w) to about 55% (w/w) water.

In various embodiments, the composition comprises:
(1) about 30% (w/w) to about 50% (w/w) of a cell paste, wherein the cell paste comprises
one or more anaerobic microorganisms;
(ii) about 0.5% (w/w) to about 5% (w/w) raftilose;
(iii) about 3% (w/w) to about 10% (w/w) maltodextrin;
(iv) about 0% (w/w) to about 0.4% (w/w) sodium alginate;
(v) about 5% (w/w) to about 10% (w/w) trehalose;
(vi) 0% (w/w) to about 5% (w/w) sucrose; and
(vii) about 35% (w/w) to about 55% (w/w) water, wherein the composition has a viscosity in the range of about 5 cP to about 6,000 cP, an osmolality in the range of about 300 Osm/L to about 4,000 Osm/L, a pH in the range of about 4 to about 8, and a redox potential in the range of from about −50 mV to about −500 mV.

In various embodiments, provided herein is a cryopreservative composition for cryopreserving anaerobic microorganisms, the composition comprising: a viscosity modifier, an energy source for the microorganisms, and an aqueous solvent, wherein the cryopreservative composition has a viscosity in the range of about 5 cP to about 6,000 cP, an osmolality in the range of about 300 Osm/L to about 4,000 Osm/L, a pH in the range of about 4 to about 8, and a redox potential in the range of from about −50 mV to about −500 mV.

In various embodiments, provided herein is a cryopreservative composition for cryopreserving anaerobic microorganisms, the composition comprising:
(i) about 3% (w/w) to about 15% (w/w) of an energy source for the microorganisms;
(ii) about 0.01% (w/w) to about 0.4% (w/w) of a viscosity modifier; and
(iii) 30% (w/w) to about 60% (w/w) of an aqueous solvent.

In various embodiments, provided herein is a cryopreservative composition for cryopreserving anaerobic microorganisms, the composition comprising:
(i) about 3% (w/w) to about 15% (w/w) of an energy source for the microorganisms;
(ii) about 0.01% (w/w) to about 0.4% (w/w) of a viscosity modifier; and
(iii) 30% (w/w) to about 60% (w/w) of an aqueous solvent, wherein the cryopreservative composition has a viscosity in the range of about 5 cP to about 6,000 cP, an osmolality in the range of about 300 Osm/L to about 4,000 Osm/L, a pH in the range of about 4 to about 8, and a redox potential in the range of from about −50 mV to about 500 mV.

In various embodiments, provided herein is a cryopreservative composition for cryopreserving anaerobic microorganisms, the composition comprising:
(1) about 30% (w/w) to about 50% (w/w) of a cell paste, wherein the cell paste comprises one or more anaerobic microorganisms;
(ii) about 3% (w/w) to about 150% (w/w) of an energy source for the microorganisms;
(iii) about 0.01% (w/w) to about 0.4% (w/w) of a viscosity modifier; and
(iv) about 30% (w/w) to about 60% (w/w) of an aqueous solvent.

In various embodiments, provided herein is a cryopreservative composition for cryopreserving anaerobic microorganisms, the composition comprising:
(i) about 30% (w/w) to about 50% (w/w) of a cell paste, wherein the cell paste comprises one or more anaerobic microorganisms;
(ii) about 3% (w/w) to about 15% (w/w) of an energy source for the microorganisms;
(iii) about 0.01% (w/w) to about 0.4% (w/w) of a viscosity modifier; and
(iv) about 30% (w/w) to about 60% (w/w) of an aqueous solvent, wherein the cryopreservative composition has a viscosity in the range of about 5 cP to about 6,000 cP, an osmolality in the range of about 300 Osm/L to about 4,000 Osm/L, a pH in the range of about 4 to about 8, and a redox potential in the range of from about −50 mV to about 500 mV.

In various embodiments, provided herein is a cryopreservative composition for cryopreserving anaerobic microorganisms, the composition comprising:
(i) about 30% (w/w) to about 50% (w/w) of a cell paste, wherein the cell paste comprises one or more anaerobic microorganisms;
(ii) about 0.5% (w/w) to about 5% (w/w) raftilose;
(iii) about 3% (w/w) to about 10% (w/w) maltodextrin;
(iv) about 0% (w/w) to about 0.4% (w/w) sodium alginate;
(v) about 5% (w/w) to about 10% (w/w) trehalose;
(vi) 0% (w/w) to about 5% (w/w) sucrose; and
(vii) about 35% (w/w) to about 55% (w/w) water.

In various embodiments, provided herein is a cry cryopreservative composition for cryopreserving anaerobic microorganisms, the composition comprising:
(i) about 30% (w/w) to about 50% (w/w) of a cell paste, wherein the cell paste comprises one or more anaerobic microorganisms;
(ii) about 0.5% (w/w) to about 5% (w/w) raftilose;
(iii) about 3% (w/w) to about 10% (w/w) maltodextrin;
(iv) about 0% (w/w) to about 0.4% (w/w) sodium alginate;
(v) about 5% (w/w) to about 10% (w/w) trehalose;
(vi) 0% (w/w) to about 5% (w/w) sucrose; and
(vii) about 35% (w/w) to about 55% (w/w) water, wherein the cryopreservative composition has a viscosity in the range of about 5 cP to about 6,000 cP, an osmolality in the range of about 300 Osm/L to about 4,000 Osm/L, a pH in the range of about 4 to about 8, and a redox potential in the range of from about −50 mV to about 500 mV.

In certain embodiments, a preserved composition described herein (e.g., a cryopreserved composition) is not a cross-linked composition or does not contain cross-linked components. In certain embodiments, the composition is not glass or glass-like.

### 2. Preserved Compositions

In one aspect, provided herein are preserved compositions generally comprising: one or more anaerobic microorganisms, a viscosity modifier, and an energy source for the microorganism. In certain embodiments, the preserved composition has a water content of less than 5% (w/w).

In various embodiments, provided herein is a preserved composition, e.g., a cryopreserved composition, comprising: one or more anaerobic microorganisms, a viscosity modifier, an energy source for the microorganism, and a water content of less than 5% (w/w).

In certain embodiments, the preserved composition is a cryopreserved composition.

In certain embodiments, the one or more anaerobic microorganisms can be facultative anaerobes, obligate anaerobes or a combination thereof. The anaerobic microorganisms can be selected from the group consisting of Bifidobacteriaceae, Coriobacteriaceae, Corynebacteriaceae, Dietziaceae, Mycobacteriaceae, Gordoniaceae, Nocardiaceae, Streptomycetaceae, Nocardiaceae 2, Propionibacterineae, Micrococcaceae, Brevibacteriaceae, Dermabacteraceae, Actinomycetaceae, Bogoriellaceae, Promicromonosporaceae, Microbacteriaceae, Cellulomonadaceae, Dermacoccaceae, Intrasporangiaceae, Geodermatophilaccae, Micromonosporaceae, Bacteroidaceae, Prevotellaceae, Porphyromonadaceae 1, Rikenellaceae, Porphyromonadaceae 2, Flavobacteriaceae, Sphingobacteriaceae, Unclassified Bacteroidetes, Cytophagaceae, Epsilonproteobacteria, Firmicutes, Lactobacillaceae, Leuconostocaceae, Aerococcaceae, Carnobacteriaceae, Aerococcaceae 2, Lachnospiraceae, Streptococcaceae, Enterococcaceae, Erysipelotrichaceae, Staphylococcaceae, Family XI Incertae Sedis, Listeriaceae, Planococcaceae 1, Family XII Incertae Sedis, Planococcaceae 2, Bacillaceae, Paenibacillaceae, Clostridiaceae (cluster I), Clostridiaceae (cluster II), Lachnospiraceae (cluster XIVa), Ruminococcaceae (cluster IV), Peptostreptococcaceae (cluster XI), Erysipelotrichaceae (clusters XVII & XVIII), Fusobacteria. Ruminococcaceae, Unclassified Clostridiales, Oscillospiraceae, Lachnospiraceae, Eubacteriaceae, Family XIII Incertae Sedis, Peptostreptococcaceae (cluster XI), Lachnospiraceae (cluster XIVa), Ruminococcaceae (cluster II), Clostridiaceae (cluster I), Erysipelotrichaceae (clusters XVII & XVIII), Peptostreplococcaceae, Family XI Incertae Sedis, Erysipelotrichi and Bacilli, Clostridia, Negativicutes, Clostridia 2, Clostridia 3/Halanaerobiales, Veillonellaceae, Acidaminococcaceae, Erysipelotrichaceae cluster XVII, Erysipelotrichaceae cluster XVIII, Erysipelotrichaceae cluster XVI, Tenericutes, Erysipelotrichaceae, Fusobacteriaceae, Leptotrichiaceae, Rhodobacteraceae, Brucellaceae, Rhizobiaceae, Phyllobacteriaceae, Aurantimonadaceae, Bradyrhizobiaceae, Methylobacteriaceae, Xanthobacteraceae, Caulobacteraceae, Hyphomicrobiaceae, Sphingomonadaceae, Acetobacteraceae, Rhodospirillaceae, Firmicutes, Comamonadaceae, Sutterellaceae, Alcaligenaceae, Burkholderiaceae 1, Oxalobacteraceae, Burkholderiaceae 2, Rhodocyclaceae, Neisseriaceae, Enterobacteriaceae, Pasteurellaceae, Vibrionaceae, Aeromonadaceae, Succinivibrionaceae, Moraxellaceae, Pseudomonadaceae, Xanthomonadaceae, Sinobacteraceae, ε-Campylobacteraceae, ε-Helicobacteraceae, ε-Campylobacteraceae 2, δ-Desulfovibrionaceae, Verrucomicrobia, Lentisphaerae, Planctomycetes, Spirochaetes, Deinococcus-Thermus, Synergistetes, Archaea/Euryarchaeota, Archaea/Crenarchaeota, Fungi-Ascomycota, Fungi-Basidiomycota, Stramenopiles, Apicomplexa, Amoebozoa, Ciliophora, Metamonada, Microsporidia, Fornicata, Akkermansiaceae, Christensenellaceae, Odoribacteraceae, Eggerthellaceae, and combinations thereof.

In certain embodiments, the one or more anaerobic microorganisms can be selected from the group consisting of Akkermansiaceae, Bacillaceae, Bacteroidaceae, Bifidobacteriaceae, Christensenellaceae, Clostridiaceae, Coriobacteriaceae, Eggerthellaceae, Enterobacteriaceae, Eubacteriaceae, Lachnospiraceae, Lactobacillaceae, Leuconostocaceae, Odoribacteraceae, Oxalobacteraceae, Porphyromonadaceae, Prevotellaceae, Rikenellaceae, Ruminococcaceae, Streptococcaceae, and combinations thereof.

In certain embodiments, the composition has a water content of less than about 5% (w/w), less than about 4.5% (w/w), less than about 4% (w/w), less than about 3.5% (w/w), less than about 3% (w/w), less than about 2.5% (w/w), less than about 2% (w/w), less than about 1.5% (w/w), less than about 1% (w/w), or less than about 0.5% (w/w).

In certain embodiments, the composition has a water content in the range of about 1% (w/w) to about 5% (w/w), about 1.5% (w/w) to about 5% (w/w), about 2% (w/w) to about 5% (w/w), about 2.5% (w/w) to about 5% (w/w), about 3% (w/w) to about 5% (w/w), about 3.5% (w/w) to about 5% (w/w), about 4% (w/w) to about 5% (w/w), about 4.5% (w/w) to about 5% (w/w), about 1% (w/w) to about 4.5% (w/w), about 1% (w/w) to about 4% (w/w), about 1/(w/w) to about 3.5% (w/w), about 1% (w/w) to about 3% (w/w), about 1% (w/w) to about 2.5% (w/w), about 1% (w/w) to about 2% (w/w), about 1% (w/w) to about 1.5% (w/w), about 1.5% (w/w) to about 4.5% (w/w), about 1.5% (w/w) to about 4% (w/w), about 1.5% (w/w) to about 3.5% (w/w), about 1.5% (w/w) to about 3% (w/w), about 1.5% (w/w) to about 2.5% (w/w), about 1.5% (w/w) to about 2% (w/w), about 2% (w/w) to about 4.5% (w/w), about 2% (w/w) to about 4% (w/w), about 2% (w/w) to about 3.5% (w/w), about 2% (w/w) to about 3% (w/w), about 2% (w/w) to about 2.5% (w/w), about 2.5% (w/w) to about 4.5% (w/w), about 2.5% (w/w) to about 4% (w/w), about 2.5% (w/w) to about 3.5% (w/w), about 2.5% (w/w) to about 3% (w/w), about 3% (w/w) to about 4.5% (w/w), about 3% (w/w) to about 4% (w/w), about 3% (w/w) to about 3.5% (w/w), about 3.5% (w/w) to about 4.5% (w/w), about 3.5% (w/w) to about 4% (w/w), or about 4% (w/w) to about 4.5% (w/w). In certain embodiments, the composition has a water content in the range of about 2% (w/w) to about 4% (w/w) or about 2% (w/w) to about 3% (w/w). In certain embodiments, the composition has a water content in the range of about 1.5% (w/w) to about 4% (w/w).

In certain embodiments, the composition has a water content of about 0.5% (w/w), about 1% (w/w), about 1.5% (w/w), about 2% (w/w), about 2.5% (w/w), about 3% (w/w), about 3.5% (w/w), about 4% (w/w), about 4.5% (w/w), or about 5% (w/w).

In certain embodiments, the viscosity modifier is a polymeric composition. In certain embodiments, the polymeric composition comprises an alginate, acacia, alginic acid, an agar, alamic acid, bentonite, carbomer, a carrageenan, a cellulose, a dextrin, a gelatin, gellan gum, guar gum, a carboxymethylcellulose, a hydroxyethylcellulose, a hydroxypropylcellulose, a methylcellulose, a mucin, pectin, a starch, xanthan gum, or a combination thereof.

In certain embodiments, the polymeric composition comprises an alginate, for example, sodium alginate. In certain embodiments, the sodium alginate has a viscosity in the range of about 250 cP to about 14,000 cP, about 500 cP to about 14,000 cP, about 750 cP to about 14,000 cP, about 1,000 cP to about 14,000 cP, about 2,000 cP to about 14,000 cP, about 3,000 cP to about 14,000 cP, about 4,000 cP to about 14,000 cP, about 5,000 cP to about 14,000 cP, about 6,000 cP to about 14,000 cP, about 7,000 cP to about 14,000 cP, about 8,000 cP to about 14,000 cP, about 9,000 cP to about 14,000 cP, about 10,000 cP to about 14,000 cP, about 11,000 cP to about 14,000 cP, about 12,000 cP to about 14,000 cP, about 13,000 cP to about 14,000 cP, about 250 cP to about 13,000 cP, about 250 cP to about 12,000 cP, about 250 cP to about 11,000 cP, about 250 cP to about 10,000 cP, about 250 cP to about 9,000 cP, about 250 cP to about 8,000 cP, about 250 cP to about 7,000 cP, about 250 cP to about 6,000 cP, about 250 cP to about 5,000 cP, about 250 cP to about 4,000 cP, about 250 cP to about 3,000 cP, about 250 cP to about 2,000 cP, about 250 cP to about 1,000 cP, about 250 cP to about 750 cP, about 250 cP to about 500 cP, about 500 cP to about 13,000 cP, about 500 cP to about 12,000 cP, about 500 cP to about 11,000 cP, about 500 cP to about 10,000 cP, about 500 cP to about 9,000 cP, about 500 cP to about 8,000 cP, about 500 cP to about 7,000 cP, about 500 cP to about 6,000 cP, about 500 cP to about 5,000 cP, about 500 cP to about 4,000 cP, about 500 cP to about 3,000 cP, about 500 cP to about 2,000 cP, about 500 cP to about 1,000 cP, about 500 cP to about 750 cP, about 750 cP to about 13,000 cP, about 750 cP to about 12,000 cP, about 750 cP to about 11,000 cP, about 750 cP to about 10,000 cP, about 750 cP to about 9,000 cP, about 750 cP to about 8,000 cP, about 750 cP to about 7,000 cP, about 750 cP to about 6,000 cP, about 750 cP to about 5,000 cP, about 750 cP to about 4,000 cP, about 750 cP to about 3,000 cP, about 750 cP to about 2,000 cP, about 750 cP to about 1,000 cP, about 1,000 cP to about 13,000 cP, about 1,000 cP to about 12,000 cP, about 1,000 cP to about 11,000 cP, about 1,000 cP to about 10,000 cP, about 1,000 cP to about 9,000 cP, about 1,000 cP to about 8,000 cP, about 1,000 cP to about 7,000 cP, about 1,000 cP to about 6,000 cP, about 1,000 cP to about 5,000 cP, about 1,000 cP to about 4,000 cP, about 1,000 cP to about 3,000 cP, about 1,000 cP to about 2,000 cP, about 2,000 cP to about 13,000 cP, about 2,000 cP to about 12,000 cP, about 2,000 cP to about 11,000 cP, about 2,000 cP to about 10,000 cP, about 2,000 cP to about 9,000 cP, about 2,000 cP to about 8,000 cP, about 2,000 cP to about 7,000 cP, about 2,000 cP to about 6,000 cP, about 2,000 cP to about 5,000 cP, about 2,000 cP to about 4,000 cP, about 2,000 cP to about 3,000 cP, about 3,000 cP to about 13,000 cP, about 3,000 cP to about 12,000 cP, about 3,000 cP to about 11,000 cP, about 3,000 cP to about 10,000 cP, about 3,000 cP to about 9,000 cP, about 3,000 cP to about 8,000 cP, about 3,000 cP to about 7,000 cP, about 3,000 cP to about 6,000 cP, about 3,000 cP to about 5,000 cP, about 3,000 cP to about 4,000 cP, about 4,000 cP to about 13,000 cP, about 4,000 cP to about 12,000 cP, about 4,000 cP to about 11,000 cP, about 4,000 cP to about 10,000 cP, about 4,000 cP to about 9,000 cP, about 4,000 cP to about 8,000 cP, about 4,000 cP to about 7,000 cP, about 4,000 cP to about 6,000 cP, about 4,000 cP to about 5,000 cP, about 5,000 cP to about 13,000 cP, about 5,000 cP to about 12,000 cP, about 5,000 cP to about 11,000 cP, about 5,000 cP to about 10,000 cP, about 5,000 cP to about 9,000 cP, about 5,000 cP to about 8,000 cP, about 5,000 cP to about 7,000 cP, about 5,000 cP to about 6,000 cP, about 6,000 cP to about 13,000 cP, about 6,000 cP to about 12,000 cP, about 6,000 cP to about 11,000 cP, about 6,000 cP to about 10,000 cP, about 6,000 cP to about 9,000 cP, about 6,000 cP to about 8,000 cP, about 6,000 cP to about 7,000 cP, about 7,000 cP to about 13,000 cP, about 7,000 cP to about 12,000 cP, about 7,000 cP to about 11,000 cP, about 7,000 cP to about 10,000 cP, about 7,000 cP to about 9,000 cP, about 7,000 cP to about 8,000 cP, about 8,000 cP to about 13,000 cP, about 8,000 cP to about 12,000 cP, about 8,000 cP to about 11,000 cP, about 8,000 cP to about 10,000 cP, about 8,000 cP to about 9,000 cP, about 9,000 cP to about 13,000 cP, about 9,000 cP to about 12,000 cP, about 9,000 cP to about 11,000 cP, about 9,000 cP to about 10,000 cP, about 10,000 cP to about 13,000 cP, about 10,000 cP to about 12,000 cP, about 10,000 cP to about 11,000 cP, about 11,000 cP to about 13,000 cP, about 11,000 cP to about 12,000 cP, or about 12,000 cP to about 13,000 cP. In certain embodiments, the sodium alginate has a viscosity range of about 250 cP to about 14000 cP.

In certain embodiments, the energy source is a cell culture media that permits growth of the microorganisms. In certain embodiments, the energy source is a monosaccharide, a disaccharide, an oligosaccharide, or a combination thereof. In certain embodiments, the monosaccharide is selected from the group consisting of glucose, fructose, xylose, and combinations thereof. In certain embodiments, the disaccharide is trehalose, sucrose, or a combination thereof. In certain embodiments, the oligosaccharide is an inulin derivative. In certain embodiments, the inulin derivative is a fructo-oligosaccharide. In certain embodiments, the fructo-oligosaccharide is raftilose. In certain embodiments, the oligosaccharide is maltodextrin.

In certain embodiments, the energy source comprises raftilose and maltodextrin.

In certain embodiments, the composition further comprises a protein stabilizer. In certain embodiments, the protein stabilizer is selected from the group consisting of an amino acid, an antioxidant, a disaccharide, a reducing agent, polyethylene glycol, and combinations thereof.

In certain embodiments, the disaccharide is selected from the group consisting of trehalose, sucrose, isomaltose, lactose, maltose, lactulose, cellobiose, and combinations thereof. In certain embodiments, the protein stabilizer comprises trehalose. In certain embodiments, the protein stabilizer comprises sucrose and trehalose.

In certain embodiments, the composition further comprises a buffering agent.

In certain embodiments, the preserved composition comprises:
(i) one or more anaerobic microorganisms;
(ii) an energy source comprising raftilose and maltodextrin;
(iii) sodium alginate: and
(iv) a protein stabilizer comprising trehalose, sucrose, and combinations thereof.

In certain embodiments, the preserved composition comprises:
(i) one or more anaerobic microorganisms;
(ii) an energy source comprising raftilose and maltodextrin;
(iii) sodium alginate;
(iv) a protein stabilizer comprising trehalose, sucrose, and combinations thereof; and
(v) a water content of less than 5% (w/w).

In certain embodiments, the preserved composition comprises:
(i) one or more anaerobic microorganisms;
(ii) an energy source comprising raftilose and maltodextrin;
(iii) sodium alginate; and
(iv) a protein stabilizer comprising trehalose, sucrose, and combinations thereof.

In certain embodiments, the preserved composition comprises:
(i) one or more anaerobic microorganisms;
(ii) an energy source comprising raftilose and maltodextrin;
(iii) sodium alginate;
(iv) a protein stabilizer comprising trehalose, sucrose, and combinations thereof; and
(v) a water content of less than 5% (w/w).

In certain embodiments, the composition, when stored at 4° C. for 6 months or 12 months and then rehydrated results in a loss of colony forming units (CFUs) by less than 3, 2, 1 or 0.5 log(s). In certain embodiments, the composition, when stored at 4° C. for 6 months or 12 months and then rehydrated results in a loss of colony forming units (CFUs) by less than 3 logs. In certain embodiments, the composition, when stored at 4° C. for 6 months or 12 months and then rehydrated results in a loss of colony forming units (CFUs) by less than 2 logs. In certain embodiments, the composition, when stored at 4° C. for 6 months or 12 months and then rehydrated results in a loss of colony forming units (CFUs) by less than 1 log. In certain embodiments, the composition, when stored at 4° C. for 6 months or 12 months and then rehydrated results in a loss of colony forming units (CFUs) by less than 0.5 log.

In certain embodiments, the composition remains substantially stable (e.g., less than a 0.5 log loss in potency (CFU/g)) at 4° C. for about 0.5 years, about 1 year, about 1.5 years, about 2 years, about 2.5 years, or about 3 years. In certain embodiments, the composition remains substantially stable at 4° C. for about 2 years.

In certain embodiments, the composition, when rehydrated in an aqueous fluid, permits regrowth of the one or more microorganisms within 2, 3, 4, or 5 hours of rehydration. In certain embodiments, the composition, when rehydrated in an aqueous fluid, permits regrowth of the one or more microorganisms within 2 hours of rehydration. In certain embodiments, the composition, when rehydrated in an aqueous fluid, permits regrowth of the one or more microorganisms within 3 hours of rehydration. In certain embodiments, the composition, when rehydrated in an aqueous fluid, permits regrowth of the one or more microorganisms within 4 hours of rehydration. In certain embodiments, the composition, when rehydrated in an aqueous fluid, permits regrowth of the one or more microorganisms within 5 hours of rehydration.

In certain embodiments, a preserved composition described herein (e.g., a cryopreserved composition) is not a cross-linked composition. In certain embodiments, the composition is not glass or glass-like.

### 3. Pharmaceutical Compositions and Food Products

In one aspect, provided herein are pharmaceutical compositions generally comprising a preserved composition or a cryopreserved composition described herein, and optionally comprising one or more pharmaceutically acceptable excipients.

In various embodiments, provided herein is a pharmaceutical composition comprising: a preserved composition (e.g., cryopreserved composition) described herein and one or more pharmaceutically acceptable excipients.

The pharmaceutical compositions provided herein can be administered by a variety of routes including, but not limited to, oral (enteral) administration, parenteral (by injection) administration, or rectal administration. In certain embodiments, the pharmaceutical compositions disclosed herein are administered orally. In certain embodiments, the pharmaceutical compositions described herein are administered by injection, e.g., via subcutaneous injection or intramuscular injection, or by injection into a tissue requiring medical intervention, e.g., cancerous tissue.

The pharmaceutical compositions provided herein may also be administered chronically (“chronic administration”). Chronic administration refers to administration of a compound or pharmaceutical composition thereof over an extended period of time, e.g., for example, over 3 months. 6 months, 1 year, 2 years, 3 years, 5 years, etc., or may be continued indefinitely, for example, for the rest of the subject's life. In certain embodiments, the chronic administration is intended to provide a constant level of one or more of the microorganisms described herein in the gastrointestinal tract of a subject.

The pharmaceutical compositions provided herein may be presented in unit dosage forms to facilitate accurate closing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pre-filled, premeasured ampules or syringes of the liquid compositions or pills, tablets, capsules or the like in the case of solid compositions.

In certain embodiments, the pharmaceutical compositions provided herein are administered to the patient as a solid dosage form. In certain embodiments, the solid dosage form is a tablet. In certain embodiments, the solid dosage form is a capsule. In certain embodiments, the solid dosage form is a caplet.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in REMINGTON: THE SCIENCE AND PRACTICE OF PHARMACY 21ST ED., Lippincott Williams & Wilkins, 2005. In certain embodiments, the pharmaceutical composition is disposed within a capsule, tablet, or caplet.

In another aspect, provided herein are food products and probiotics generally comprising a preserved composition or a cryopreserved composition described herein. In certain embodiments, the food product or probiotic is disposed within a capsule, tablet, or caplet.

Under certain circumstances, it may be desirable to administer the pharmaceutical composition or the food product/probiotic by oral delivery. In order to deliver the microorganisms to the intestinal tract having the appropriate environment for recovery and growth of the microorganisms (e.g., redox potential), it may be desirable to coat the capsule, tablet or caplet with a functional coating, e.g., a coating that releases the microorganisms in a suitable environment (e.g., an environment with the appropriate pH and/or redox potential) to facilitate recovery and growth of the microorganisms).

For example, the capsule, tablet or caplet may be coated with a delayed release coating, such as an enteric coating. Enteric coatings are generally acid resistant that protect the components of a dosage form from being released in the stomach but yet dissolve or disintegrate in neutral or mildly alkaline conditions that are typically encountered after the stomach. Exemplary coatings including hydroxypropyl methylcellulose phthalate, methacrylic acid/methu methyl methacylate polymers (e.g. Eudragit® materials such as Eudragit® L100-55, Eudragit® L30D, and Eudragit® S-100), cellulose acetate phthalate, cellulose acetate, trimellitate, hydroxypropylmethylcellulose phthalate, hydroxypropylmethylcellulose acetate succinate, poly (MA-EA)*1:1, poly (MA-MMA)*1:1, poly (MA-MMA)*1:2, HP-F, sureteric, acryl-Eze, Advantia performance, aquateric, aquocoat ECD, aqoat, CAP, CAT and polyvinyl acetate phthalate (see, e.g. U.S. Pat. No. 9,629,803 B2 and US2018/0325825). These coated capsules, tablets and caplets can keep their contents dry until the capsules, tablets and caplets reach the appropriate environment, e.g., pH and/or redox potential, whereupon the coatings break down and release the microorganisms into the desired environment in the subject to facilitate recovery and growth.

Furthermore, depending upon the circumstances, the cryopreserved organisms can be mixed with a carbonated solution prior to ingestion. When ingested without food (e.g., on an empty stomach), the organisms may have a short resistance time in the stomach and enter the intestinal tract within 30 minutes of ingestion.

### 4. Methods of Makin Cryopreserved Compositions of Anaerobic Microorganisms

The disclosure also provides methods of producing a cryopreserved composition described herein.

In certain embodiments, the methods generally comprise two steps. A first step involves combining anaerobic microorganisms with an excipient blend comprising an aqueous mixture comprising a viscosity modifier, and an energy source for the microorganisms to produce a cryopreservative mixture, wherein the cryopreservative mixture has a viscosity in the range of about 5 cP to about 6,000 cP, an osmolality in the range of about 300 Osm/L to about 4,000 Osm/L, a pH in the range of about 4 to about 8 and optionally a redox potential in the range of 50 mV to about −500 mV. Thereafter, in a separate step, the cryopreservative mixture is freeze dried to reduce the water content of the cryopreservative mixture to less than about 5% (w/w) to produce the cryopreserved composition.

In certain embodiments, in the first step (step (a)), the anaerobic microorganisms are selected from the group consisting of Bifidobacteriaceae, Coriobacteriaceae, Corynebacteriaceae, Dietziaceae, Mycobacteriaceae, Gordoniaceae, Nocardiaceae, Streptomycetaceae, Nocardiaceae 2, Propionibacterineae, Micrococcaceae, Brevibacteriaceae, Dermabacteraceae, Actinomycetaceae, Bogoriellaceae, Promicromonosporaceae, Microbacteriaceae, Cellulomonadaceae, Dermacoccaceae, Intrasporangiaceae, Geodermatophilaceae, Micromonosporaceae, Bacteroidaceae, Prevotellaceae, Porphyromonadaceae 1, Rikenellaceae, Porphyromonadaceae 2, Flavobacteriaceae, Sphingobacteriaceae, Unclassified Bacteroidetes, Cytophagaceae, Epsilonproteobacteria, Firmicutes, Lactobacillaceae, Leuconostocaceae, Aerococcaceae, Carnobacteriaceae, Aerococcaceae 2, Lachnospiraceae, Streptococcaceae, Enterococcaceae, Erysipelotrichaceae, Staphylococcaceae, Family XI Incertae Sedis, Listeriaceae, Planococcaceae 1, Family XI Incertae Sedis, Planococcaceae 2, Bacillaceae, Paenibacillaceae, Clostridiaceae (cluster I), Clostridiaceae (cluster II), Lachnospiraceae (cluster XIVa), Ruminococcaceae (cluster IV), Peptostreptococcaceae (cluster I), Erysipelotrichaceae (clusters XVII & XVIII), Fusobacteria, Ruminococcaceae, Unclassified Clostridiales, Oscillospiraceae, Lachnospiraceae, Eubacteriaceae, Family XIII Incertae Sedis, Peptostreptococcaceae (cluster XI), Lachnospiraceae (cluster XIVa), Ruminococcaceae (cluster II), Clostridiaceae (cluster I), Erysipelotrichaceae (clusters XVII & XVIII), Peptostreptococcaceae, Family XI Incertae Sedis, Erysipelotrichi and Bacilli, Clostridia, Negativicutes, Clostridia 2, Clostridia 3/Halanaerobiales, Veillonellaceae, Acidaminococcaceae, Erysipelotrichaceae cluster XVII, Erysipelotrichaceae cluster XVIII, Erysipelotrichaceae cluster XVI, Tenericutes, Erysipelotrichaceae, Fusobacteriaceae, Leptotrichiaceae, Rhodobacteraceae, Brucellaceae, Rhizobiaceae, Phyllobacteriaceae, Aurantimonadaceae, Bradyrhizobiaceae, Methylobacteriaceae, Xanthobacteraceae, Caulobacteraceae, Hyphomicrobiaceae, Sphingomonadaceae, Acetobacteraceae, Rhodospirillaceae, Firmicutes, Comamonadaceae, Sutterellaceae, Alcaligenaceae, Burkholderiaceae 1, Oxalobacteraceae, Burkholderiaceae 2, Rhodocyclaceae, Neisseriaceae, Enterobacteriaceae, Pasteurellaceae, Vibrionaceae, Aeromonadaceae, Succinivibrionaceae, Moraxellaceae, Pseudomonadaceae, Xanthomonadaceae, Sinobacteraceae, ε-Campylobacteraceae 1, Helicobacteraceae, ε-Campylobacteraceae 2, δ-Desulfovibrionaceae, Verrucomicrobia, Lentisphaerae, Planctomycetes, Spirochaetes, Deinococcus-Thermus, Synergisteles, Archaea/Euryarchaeota, Archaea/Crenarchaeota, Fungi Ascomycota, Fungi-Basidiomycota, Stramenopiles, Apicomplexa, Amoebozoa, Ciliophora, Metamonada, Microsporidia, Fornicata, Akkermansiaceae, Christensenellaceae, Odoribacteraceae, Eggerthellaceae, and combinations thereof.

In certain embodiments, during step (a), the anaerobic microorganisms are selected from the group consisting of Akkermansiaceae, Bacillaceae, Bacteroidaceae, Bifidobacteriaceae, Christensenellaceae, Clostridiaceae, Coriobacteriaceae, Eggerthellaceae, Enterobacteriaceae, Eubacteriaceae, Lachnospiraceae, Lactobacillaceae, Leuconostocaceae, Odoribacteraceae, Oxalobacteraceae, Porphyromonadaceae, Prevotellaceae, Rikenellaceae, Ruminococcaceae, Streptococcaceae, and combinations thereof.

In certain embodiments, during step (a), the microorganisms are in the form of a cell paste. In certain embodiments, the cell paste comprises the microorganisms in a cell culture media. The cell paste can be prepared by cultivating the microorganisms and then harvesting the microorganisms, e.g., to remove water content, in order to form the cell paste.

In certain embodiments, during step (a), the cell paste is not washed, e.g., not washed with water, a buffer or salt solution, to remove cell culture media prior to combining with the excipient blend.

In certain embodiments, during step (a), the cryopreservative mixture comprises about 30% (w/w) to about 50% (w/w), about 32% (w/w) to about 50% (w/w), about 34% (w/w) to about 50% (w/w), about 36% (w/w) to about 50% (w/w), about 38% (w/w) to about 50% (w/w), about 40% (w/w) to about 50% (w/w), about 42% (w/w) to about 50% (w/w), about 44% (w/w) to about 50% (w/w), about 46% (w/w) to about 50% (w/w), about 48% (w/w) to about 50% (w/w), about 30% (w/w) to about 48% (w/w), about 30% (w/w) to about 46% (w/w), about 30% (w/w) to about 44% (w/w), about 30% (w/w) to about 42% (w/w), about 30% (w/w) to about 40% (w/w), about 30% (w/w) to about 38% (w/w), about 30% (w/w) to about 36% (w/w), about 30% (w/w) to about 34% (w/w), about 30% (w/w) to about 32% (w/w), about 32% (w/%) to about 48% (w/w), about 32% (w/w) to about 46% (w/w), about 32% (w/w) to about 44% (w/w), about 32% (w/w) to about 42% (w/w), about 32% (w/w) to about 40% (w/w), about 32% (w/w) to about 38% (w/w), about 32% (w/w) to about 36% (w/w), about 32% (w/w) to about 34% (w/w), about 34% (w/w) to about 48% (w/w), about 34% (w/w) to about 46% (w/w), about 34% (w/w) to about 44% (w/w), about 34% (w/w) to about 42% (w/w), about 34% (w/w) to about 40% (w/w), about 34% (w/w) to about 38% (w/w), about 34% (w/w) to about 36% (w/w), about 36% (w/w) to about 48% (w/w), about 36% (w/w) to about 46% (w/w), about 36% (w/w) to about 44% (w/w), about 36% (w/w) to about 42% (w/w), about 36% (w/w) to about 40% (w/w), about 36% (w/w) to about 38% (w/w), about 38% (w/w) to about 48% (w/w), about 38% (w/w) to about 46% (w/w), about 38% (w/w) to about 44% (w/w), about 38% (w/w) to about 42% (w/w), about 38% (w/w) to about 40% (w/w), about 40% (w/w) to about 48% (w/w), about 40% (w/w) to about 46% (w/w), about 40% (w/w) to about 44% (w/w), about 40% (w/w) to about 42% (w/w), about 42% (w/w) to about 48% (w/w), about 42% (w/w) to about 46% (w/w), about 42% (w/w) to about 44% (w/w), about 44% (w/w) to about 48% (w/w), about 44% (w/w) to about 46% (w/w), or about 46% (w/w) to about 48% (w/w), of the cell paste or cell slurry, when solvated in an aqueous solvent. In certain embodiments, in step (a), the cryopreservative mixture comprises about 30% (w/w) to about 50% (w/w) of the cell paste or cell slurry, when solvated in an aqueous solvent.

In certain embodiments, during step (a), the cryopreservative mixture comprises about 30% (w/w), about 31% (w/w), about 32% (w/w), about 33% (w/w), about 34% (w/w), about 35% (w/w), about 36% (w/w), about 37% (w/w), about 38% (w/w), about 39% (w/w), about 40% (w/w), about 41% (w/w), about 42% (w/w), about 43% (w % w), about 44% (w/w), about 45% (w/w), about 46% (w/w), about 47% (w/w), about 48% (w/w), about 49% (w/w), or about 50% (w/w) of the cell paste or cell slurry, when solvated in an aqueous solvent, e.g. water (e.g., ionized or deionized water).

In certain embodiments, during step (a), the aqueous mixture comprises deionized water. In certain embodiments, in step (a), the cryopreservative mixture comprises about 30% (w/w) to about 60% (w/w), about 35% (w/w) to about 60% (w/w), about 40% (w/w) to about 60% (w/w), about 45% (w/w) to about 60% (w/w), about 50% (w/w) to about 60% (w/w), about 55% (w/w) to about 60% (w/w), about 30% (w/w) to about 55% (w/w), about 30% (w/w) to about 50% (w/w), about 30% (w/w) to about 45% (w/w), about 30% (w/w) to about 40% (w/w), about 30% (w/w) to about 35% (w/w), about 35% (w/w) to about 55% (w/w), about 35% (w/w) to about 50% (w/w), about 35% (w/w) to about 45% (w/w), about 35% (w/w) to about 40% (w/w), about 40% (w/w) to about 55% (w/w), about 40% (w/w) to about 50% (w/w), about 40% (w/w) to about 45% (w/w), about 45% (w/w) to about 55% (w/w), about 45% (w/w) to about 50% (w/w), or about 50% (w/w) to about 55% (w/w) water.

In certain embodiments, during step (a), the cryopreservative mixture comprises about 30% (w/w), about 31% (w/w), about 32% (w/w), about 33% (w/w), about 34% (w/w), about 35% (w/w), about 36% (w/w), about 37% (w/w), about 38% (w/w), about 39% (w/w), about 40% (w/w), about 41% (w/w), about 42% (w/w), about 43% (w % w), about 44% (w/w), about 45% (w/w), about 46% (w/w), about 47% (w/w), about 48% (w/w), about 49% (w/w), about 50% (w/w), about 51% (w/w), about 52% (w/w), about 53% (w/w), about 54% (w/w), about 55% (w/w), about 56% (w/w), about 57% (w/w), about 58% (w/w), about 59% (w/w), or about 60% (w/w) water.

In certain embodiments, during step (a), the cryopreservative mixture comprises about 0.01% (w/w) to about 0.4% (w/w), about 0.05% (w/w) to about 0.4% (w/w), about 0.1% (w/w) to about 0.4% (w/w), about 0.15% (w/w) to about 0.4% (w/w), about 0.2% (w/w) to about 0.4% (w/w), about 0.25% (w/w) to about 0.4% (w/w), about 0.3% (w/w) to about 0.4% (w/w), about 0.35% (w/w) to about 0.4% (w/w), about 0.01% (w/w) to about 0.35% (w/w), about 0.01% (w/w) to about 0.3% (w/w), about 0.01% (w/w) to about 0.25% (w/w), about 0.01% (w/w) to about 0.2% (w/w), about 0.01% (w/w) to about 0.15% (w/w), about 0.01% (w/w) to about 0.1% (w/w), about 0.01% (w/w) to about 0.05% (w/w), about 0.05% (w/w) to about 0.35% (w/w), about 0.05% (w/w) to about 0.3% (w/w), about 0.05% (w/w) to about 0.25% (w/w), about 0.05% (w/w) to about 0.2% (w/w), about 0.05% (w/w) to about 0.15% (w/w), about 0.05% (w/w) to about 0.1% (w/w), about 0.1% (w/w) to about 0.35% (w/w), about 0.1% (w/w) to about 0.3% (w/w), about 0.1% (w/w) to about 0.25% (w/w), about 0.1% (w/w) to about 0.2% (w/w), about 0.1% (w/w) to about 0.15% (w/w), about 0.15% (w/w) to about 0.35% (w/w), about 0.15% (w/w) to about 0.3% (w/w), about 0.15% (w/w) to about 0.25% (w/w), about 0.15% (w/w) to about 0.2% (w/w), about 0.2% (w/w) to about 0.35% (w/w), about 0.2% (w/w) to about 0.3% (w/w), about 0.2% (w/w) to about 0.25% (w/w), about 0.25% (w/w) to about 0.35% (w/w), about 0.25% (w/w) to about 0.3% (w/w), or about 0.3% (w/w) to about 0.35% (w/w) of the viscosity modifier. In certain embodiments, in step (a), the cryopreservative mixture comprises about 0.01% (w/w) to about 0.4% (w/w) of the viscosity modifier.

In certain embodiments, during step (a), the cryopreservative mixture comprises about 0.01% (w/w), about 0.02% (w/w), about 0.03% (w/w), about 0.04% (w/w), about 0.05% (w/w), about 0.06% (w/w), about 0.07% (w/w), about 0.08% (w/w), about 0.09% (w/w), about 0.1% (w/w), about 0.11% (w/w), about 0.12% (w/w), about 0.13% (w/w), about 0.14% (w/w), about 0.15% (w/w), about 0.16% (w/w), about 0.17% (w/w), about 0.18% (w/w), about 0.19% (w/w), about 0.2% (w/w), about 0.21% (w/w), about 0.22% (w/w), about 0.23% (w/w), about 0.24% (w/w), about 0.25% (w/w), about 0.26% (w/w), about 0.27% (w/w), about 0.28% (w/w), about 0.29% (w/w), about 0.3% (w/w), about 0.31% (w/w), about 0.32% (w/w), about 0.33% (w/w), about 0.34% (w/w), about 0.35% (w/w), about 0.36% (w/w), about 0.37% (w/w), about 0.38% (w/w), about 0.39% (w/w), or about 0.4% (w/w), of the viscosity modifier.

In certain embodiments, during step (a), the viscosity modifier is a polymeric composition. In certain embodiments, the polymeric composition comprises an alginate, acacia, alginic acid, an agar, alamic acid, bentonite, carbomer, a carrageenan, a cellulose, a dextrin, a gelatin, gellan gum, guar gum, a carboxymethylcellulose, a hydroxyethylcellulose, a hydroxypropylcellulose, a methylcellulose, a mucin, pectin, a starch, xanthan gum, or a combination thereof.

In certain embodiments, the polymeric composition comprises an alginate, e.g., sodium alginate. The may have a viscosity in the range of about 250 cP to about 14,000 cP, about 500 cP to about 14,000 cP, about 750 cP to about 14,000 cP, about 1,000 cP to about 14,000 cP, about 2,000 cP to about 14,000 cP, about 3,000 cP to about 14,000 cP, about 4,000 cP to about 14,000 cP, about 5,000 cP to about 14,000 cP, about 6,000 cP to about 14,000 cP, about 7,000 cP to about 14,000 cP, about 8,000 cP to about 14,000 cP, about 9,000 cP to about 14,000) cP, about 10,000 cP to about 14,000 cP, about 11,000 cP to about 14,000 cP, about 12,000 cP to about 14,000 cP, about 13,000 cP to about 14,000 cP, about 250 cP to about 13,000 cP, about 250 cP to about 12,100 cP, about 250 cP to about 11,000 cP, about 250 cP to about 10,000 cP, about 250 cP to about 9,000 cP, about 250 cP to about 8,000 cP, about 250 cP to about 7,000 cP, about 250 cP to about 6,000 cP, about 250 cP to about 5,000 cP, about 250 cP to about 4,000 cP, about 250 cP to about 3,000 cP, about 250 cP to about 2,000 cP, about 250 cP to about 1,000 cP, about 250 cP to about 750 cP, about 250 cP to about 500 cP, about 500 cP to about 13,000 cP, about 500 cP to about 12,000 cP, about 500 cP to about 11,000 cP, about 500 cP to about 10,000 cP, about 500 cP to about 9,000 cP, about 500 cP to about 8,000 cP, about 500 cP to about 7,000 cP, about 500 cP to about 6,000 cP, about 500 cP to about 5,000 cP, about 500 cP to about 4,000 cP, about 500 cP to about 3,000 cP, about 500 cP to about 2,000 cP, about 500 cP to about 1,000 cP, about 500 cP to about 750 cP, about 750 cP to about 13,000 cP, about 750 cP to about 12,000 cP, about 750 cP to about 11,000 cP, about 750 cP to about 10,000 cP, about 750 cP to about 9,000 cP, about 750 cP to about 8,000 cP, about 750 cP to about 7,000 cP, about 750 cP to about 6,000 cP, about 750 cP to about 5,000 cP, about 750 cP to about 4,000 cP, about 750 cP to about 3,000 cP, about 750 cP to about 2,000 cP, about 750 cP to about 1,000 cP, about 1,000 cP to about 13,000 cP, about 1,000 cP to about 12,100 cP, about 1,000 cP to about 11,000 cP, about 1,000 cP to about 10,000 cP, about 1,000 cP to about 9,000 cP, about 1,000 cP to about 8,000 cP, about 1,000 cP to about 7,000 cP, about 1,000 cP to about 6,000 cP, about 1,000 cP to about 5,000 cP, about 1,000 cP to about 4,000 cP, about 1,000 cP to about 3,000 cP, about 1,000 cP to about 2,000 cP, about 2,000 cP to about 13,000 cP, about 2,000 cP to about 12,000 cP, about 2,000 cP to about 11,000 cP, about 2,000 cP to about 10,000 cP, about 2,000 cP to about 9,000 cP, about 2,000 cP to about 8,000 cP, about 2,000 cP to about 7,000 cP, about 2,000 cP to about 6,000 cP, about 2,000 cP to about 5,000 cP, about 2,000 cP to about 4,000 cP, about 2,000 cP to about 3,000 cP, about 3,000 cP to about 13,000 cP, about 3,000 cP to about 12,000 cP, about 3,000 cP to about 11,000 cP, about 3,000 cP to about 10,000 cP, about 3,000 cP to about 9,000 cP, about 3,000 cP to about 8,000 cP, about 3,000 cP to about 7,000 cP, about 3,000 cP to about 6,000 cP, about 3,000 cP to about 5,000 cP, about 3,000 cP to about 4,000 cP, about 4,000 cP to about 13,000 cP, about 4,000 cP to about 12,000 cP, about 4,000 cP to about 11,000 cP, about 4,000 cP to about 10,000 cP, about 4,000 cP to about 9,000 cP, about 4,000 cP to about 8,000 cP, about 4,000 cP to about 7,000 cP, about 4,000 cP to about 6,000 cP, about 4,000 cP to about 5,000 cP, about 5,000 cP to about 13,000 cP, about 5,000 cP to about 12,000 cP, about 5,000 cP to about 11,000 cP, about 5,000 cP to about 10,000 cP, about 5,000 cP to about 9,000 cP, about 5,000 cP to about 8,000 cP, about 5,000 cP to about 7,000 cP, about 5,000 cP to about 6,000 cP, about 6,000 cP to about 13,000 cP, about 6,000 cP to about 12,000 cP, about 6,000 cP to about 11,000 cP, about 6,000 cP to about 10,000 cP, about 6,000 cP to about 9,000 cP, about 6,000 cP to about 8,000 cP, about 6,000 cP to about 7,000 cP, about 7,000 cP to about 13,000 cP, about 7,000 cP to about 12,000 cP, about 7,000 cP to about 11,000 cP, about 7,000 cP to about 10,000 cP, about 7,000 cP to about 9,000 cP, about 7,000 cP to about 8,000 cP, about 8,000 cP to about 13,000 cP, about 8,000 cP to about 12,000 cP, about 8,000 cP to about 11,000 cP, about 8,000 cP to about 10,000 cP, about 8,000 cP to about 9,000 cP, about 9,000 cP to about 13,000 cP, about 9,000 cP to about 12,000 cP, about 9,000 cP to about 11,000 cP, about 9,000 cP to about 10,000 cP, about 10,000 cP to about 13,000 cP, about 10,000 cP to about 12,000 cP, about 10,000 cP to about 11,000 cP, about 11,000 cP to about 13,000 cP, about 11,000 cP to about 12,000 cP, or about 12,000 cP to about 13,000 cP. In certain embodiments, the sodium alginate has a viscosity range of about 250 cP to about 14000 cP.

In certain embodiments, during step (a), the cryopreservative mixture comprises about 3% (w/w) to about 15% (w/w), about 5% (w/w) to about 15% (w/w), about 7% (w/w) to about 15% (w/w), about 9% (w/w) to about 15% (w/w), about 11% (w/w) to about 15% (w/w), about 13% (w/w) to about 15% (w/w), about 3% (w/w) to about 13% (w/w), about 3% (w/w) to about 11% (w/w), about 3% (w/w) to about 9% (w/w), about 3% (w/w) to about 7% (w/w), about 3% (w/w) to about 5% (w/w), about 5% (w/w) to about 13% (w/w), about 5% (w/w) to about 11% (w/w), about 5% (w/w) to about 9% (w/w), about 5% (w/w) to about 7% (w/w), about 7% (w/w) to about 13% (w/w), about 7% (w/w) to about 11% (w/w), about 7% (w/w) to about 9% (w/w), about 9% (w/w) to about 13% (w/w), about 9% (w/w) to about 11% (w/w), or about 11% (w/w) to about 13% (w/w), of the energy source. In certain embodiments, in step (a), the cryopreservative mixture comprises about 3% (w/w) to about 15% (w/w) of the energy source.

In certain embodiments, during step (a), the cryopreservative mixture comprises about 3% (w/w), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), or about 15% (w/w) of the energy source.

In certain embodiments, during step (a), the energy source is a monosaccharide (e.g., glucose, fructose, xylose, and combinations thereof), a disaccharide (e.g., trehalose, sucrose, or a combination thereof), an oligosaccharide (e.g. maltodextrin or an inulin derivative (e.g., a fructo-oligosaccharide such as raftilose), or a combination thereof. In certain embodiments, during step (a), the energy source comprises raftilose and maltodextrin.

In certain embodiments, during step (a), the cryopreservative mixture comprises about 0.5% (w/w) to about 5% (w/w), about 1% (w/w) to about 5% (w/w), about 1.5% (w/w) to about 5% (w/w), about 2% (w/w) to about 5% (w/w), about 2.5% (w/w) to about 5% (w/w), about 3% (w/w) to about 5% (w/w), about 3.5% (w/w) to about 5% (w/w), about 4% (w/w) to about 5% (w/w), about 4.5% (w/w) to about 5% (w/w), about 0.5% (w/w) to about 4.5% (w/w), about 0.5% (w/w) to about 4% (w/w), about 0.5% (w/w) to about 3.5% (w/w), about 0.5% (w/w) to about 3% (w/w), about 0.5% (w/w) to about 2.5% (w/w), about 0.5% (w/w) to about 2% (w/w), about 0.5% (w/w) to about 1.5% (w/w), about 0.5% (w/w) to about 1% (w/w), about 1% (w/w) to about 4.5% (w/w), about 1 (w/w) to about 4% (w/w), about 1% (w/w) to about 3.5% (w/w), about 1% (w/w) to about 3% (w/w), about 1 (w/w) to about 2.5% (w/w), about 1% (w/w) to about 2% (w/w), about 1% (w/w) to about 1.5% (w/w), about 1.5% (w/w) to about 4.5% (w/w), about 1.5% (w/w) to about 4% (w/w), about 1.5% (w/w) to about 3.5% (w/w), about 1.5% (w/w) to about 3% (w/w), about 1.5% (w/w) to about 2.5% (w/w), about 1.5% (w/w) to about 2% (w/w), about 2% (w/w) to about 4.5% (w/w), about 2% (w/w) to about 4% (w/w), about 2% (w/w) to about 3.5% (w/w), about 2% (w/w) to about 3% (w/w), about 2% (w/w) to about 2.5% (w/w), about 2.5% (w/w) to about 4.5% (w/w), about 2.5% (w/w) to about 4% (w/w), about 2.5% (w/w) to about 3.5% (w/w), about 2.5% (w/w) to about 3% (w/w), about 3% (w/w) to about 4.5% (w/w), about 3% (w/w) to about 4% (w/w), about 3% (w/w) to about 3.5% (w/w), about 3.5% (w/w) to about 4.5% (w/w), about 3.5% (w/w) to about 4% (w/w), or about 4% (w/w) to about 4.5% (w/w) raftilose. In certain embodiments, during step (a), the cryopreservative mixture comprises about 0.5% (w/w) to about 5% (w/w) raftilose.

In certain embodiments, during step (a), the cryopreservative composition comprises about 0.5% (w/w), about 1% (w/w), about 1.5% (w/w), about 2% (w/w), about 3% (w/w), about 3.5% (w/w), about 4% (w/w), about 4.5% (w/w), or about 5% (w/w) raftilose.

In certain embodiments, during step (a), the cryopreservative mixture comprises about 3% (w/w) to about 10% (w/w), about 4% (w/w) to about 10% (w/w), about 5% (w/w) to about 10% (w/w), about 6% (w/w) to about 10% (w/w), about 7% (w/w) to about 10% (w/w), about 8% (w/w) to about 10% (w/w), about 9% (w/w) to about 10% (w/w), about 3% (w/w) to about 9% (w/w), about 3% (w/w) to about 8% (w/w), about 3% (w/w) to about 7% (w/w), about 3% (w/w) to about 6% (w/w), about 3% (w/w) to about 5% (w/w), about 3% (w/w) to about 4% (w/w), about 4% (w/w) to about 9% (w/w), about 4% (w/w) to about 8% (w/w), about 4% (w/w) to about 7% (w/w), about 4% (w/w) to about 6% (w/w), about 4% (w/w) to about 5% (w/w), about 5% (w/w) to about 9% (w/w), about 5% (w/w) to about 8% (w/w), about 5% (w/w) to about 7% (w/w), about 5% (w/w) to about 6% (w/w), about 6% (w/w) to about 9% (w/w), about 6% (w/w) to about 8% (w/w), about 6% (w/w) to about 7% (w/w), about 7% (w/w) to about 9% (w/w), about 7% (w/w) to about 8% (w/w), or about 8% (w/w) to about 9% (w/w) maltodextrin. In certain embodiments, during step (a), the cryopreservative mixture comprises about 3% (w/w) to about 10% (w/w) maltodextrin.

In certain embodiments, during step (a), the cryopreservative mixture about 3% (w/w), about 3.5% (w/w), about 4% (w/w), about 4.5% (w/w), about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) maltodextrin.

In certain embodiments, during step (a), the aqueous mixture further comprises a protein stabilizer. In certain embodiments, in step (a), the cryopreservative mixture comprises about 5% (w/w) to about 10% (w/w), about 6% (w/w) to about 10% (w/w), about 7% (w/w) to about 10% (w/w), about 8% (w/w) to about 10% (w/w), about 9% (w/w) to about 10% (w/w), about 5% (w/w) to about 9% (w/w), about 5% (w/w) to about 8% (w/w), about 5% (w/w) to about 7% (w/w), about 5% (w/w) to about 6% (w/w), about 6% (w/w) to about 9% (w/w), about 6% (w/w) to about 8% (w/w), about 6% (w/w) to about 7% (w/w), about 7% (w/w) to about 9% (w/w), about 7% (w/w) to about 8% (w/w), or about 8% (w/w) to about 9% (w/w) of the protein stabilizer.

In certain embodiments, during step (a), the cryopreservative mixture comprises about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) of the protein stabilizer.

In certain embodiments, the protein stabilizer is selected from the group consisting of an amino acid, an antioxidant, a disaccharide, a reducing agent, polyethylene glycol, and combinations thereof. The disaccharide can be selected from the group consisting of trehalose, sucrose, isomaltose, lactose, maltose, lactulose, cellobiose, and combinations thereof. In certain embodiments, the protein stabilizer comprises trehalose. In certain embodiments, the protein stabilizer comprises sucrose and trehalose.

In certain embodiments, during step (a), the cryopreservative mixture comprises about 5% (w/w) to about 10% (w/w), about 6% (w/w) to about 10% (w/w), about 7% (w/w) to about 10% (w/w), about 8% (w/w) to about 10% (w/w), about 9% (w/w) to about 10% (w/w), about 5% (w/w) to about 9% (w/w), about 5% (w/w) to about 8% (w/w), about 5% (w/w) to about 7% (w/w), about 5% (w/w) to about 6% (w/w), about 6% (w/w) to about 9% (w/w), about 6% (w/w) to about 8% (w/w), about 6% (w/w) to about 7% (w/w), about 7% (w/w) to about 9% (w/w), about 7% (w/w) to about 8% (w/w), or about 8% (w/w) to about 9% (w/w) trehalose. In certain embodiments, during step (a), the cryopreservative mixture comprises about 5% (w/w) to about 10% (w/w) trehalose.

In certain embodiments, during step (a), the cryopreservative mixture comprises about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) trehalose.

In certain embodiments, during step (a), the cryopreservative mixture comprises 0% (w/w) to about 10% (w/w), about 1% (w/w) to about 10% (w/w), about 2% (w/w) to about 10% (w/w), about 3% (w/w) to about Its % (w/w), about 4% (w/w) to about 10% (w/w), about 5% (w/w) to about 10% (w/w), about 6% (w/w) to about 10% (w % w), about 7% (w/w) to about 10% (w/w), about 8% (w/w) to about 10% (w/w), about 9% (w/w) to about 10% (w/w), 0% (w/w) to about 9% (w/w), 0% (w/w) to about 8% (w/w), 0% (w/w) to about 7% (w/w), 0% (w/w) to about 6% (w/w), 0% (w/w) to about 5% (w/w), 0% (w/w) to about 4% (w/w), 0% (w/w) to about 3% (w/w), 0% (w/w) to about 2% (w/w), 0% (w/w) to about 1% (w/w), about 1% (w/w) to about 9% (w/w), about 1% (w/w) to about 8% (w/w), about 1(w/w) to about 7% (w/w), about 1% (w/w) to about 6% (w/w), about 1% (w/w) to about 5% (w/w), about 1 (w/w) to about 4% (w/w), about 1% (w/w) to about 3% (w/w), about 1% (w/w) to about 2% (w/w), about 2% (w/w) to about 99% (w/w), about 2% (w/w) to about 8% (w/w), about 2% (w/w) to about 7% (w/w), about 2% (w/w) to about 6% (w/w), about 2% (w/w) to about 5% (w/w), about 2% (w/w) to about 4% (w/w), about 2% (w/w) to about 3% (w/w), about 3% (w/w) to about 9% (w/w), about 3% (w/w) to about 8% (w/w), about 3% (w/w) to about 7% (w/w), about 3% (w/w) to about 6% (w/w), about 3% (w/w) to about 5% (w/w), about 3% (w/w) to about 4% (w/w), about 4% (w/w) to about 9% (w/w), about 4% (w/w) to about 8% (w/w), about 4% (w/w) to about 7% (w/w), about 4% (w/w) to about 6% (w/w), about 4% (w/w) to about 5% (w/w), about 5% (w/w) to about 9% (w/w), about 5% (w/w) to about 8% (w/w), about 5% (w/w) to about 7% (w/w), about 5% (w/w) to about 6% (w/w), about 6% (w/w) to about 9% (w/w), about 6% (w/w) to about 8% (w/w), about 6% (w/w) to about 7% (w/w), about 7% (w/w) to about 9% (w/w), about 7% (w/w) to about 8% (w/w), or about 8% (w/w) to about 9% (w/w) sucrose. In certain embodiments, during step (a), the cryopreservative mixture comprises about 5% (w/w) to about 10% (w/w) sucrose, when solvated in an aqueous solvent. In certain embodiments, during step (a), the cryopreservative mixture comprises 0% (w/w) to about 5% (w/%) sucrose.

In certain embodiments, during step (a), the cryopreservative mixture comprises about 0.5% (w/w), about 1% (w/w), about 1.5% (w/w), about 2% (w/w), about 2.5% (w/w), about 3% (w/w), about 3.S % (w/w), about 4% (w/w), about 4.5% (w/w), about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) sucrose.

In certain embodiments, during step (a), the aqueous mixture further comprises a buffering agent, e.g., a sodium phosphate buffering system or a potassium phosphate buffering system.

In certain embodiments, during step (a), the cryopreservative mixture has a viscosity in the range of about 5 cP to about 6,000 cP, about 50 cP to about 6,000 cP, about 100 cP to about 6,000 cP, about 200 cP to about 6,000 cP, about 300 cP to about 6,000 cP, about 400 cP to about 6,000 cP, about 500 cP to about 6,000 cP, about 1,000 cP to about 6,000 cP, about 1,500 cP to about 6,000 cP, about 2,000 cP to about 6,000 cP, about 2,500 cP to about 6,000 cP, about 3,000 cP to about 6,000 cP, about 3,500 cP to about 6,000 cP, about 4,000 cP to about 6,000 cP, about 4,500 cP to about 6,000 cP, about 5,000 cP to about 6,000 cP, about 5,500 cP to about 6,000 cP, about 5 cP to about 5,500 cP, about 5 cP to about 5,000 cP, about 5 cP to about 5,000 cP, about 5 cP to about 4,500 cP, about 5 cP to about 4,000 cP, about 5 cP to about 3,500 cP, about 5 cP to about 3,000 cP, about 5 cP to about 2,500 cP, about 5 cP to about 2,000 cP, about 5 cP to about 1,500 cP, about 5 cP to about 1,000 cP, about 5 cP to about 500 cP, about 5 cP to about 400 cP, about 5 cP to about 300 cP, about 5 cP to about 200 cP, about 5 cP to about 100 cP, about 5 cP to about 50 cP, about 50 cP to about 5,500 cP, about 50 cP to about 5,000 cP, about 50 cP to about 4,500 cP, about 50 cP to about 4,000 cP, about 50 cP to about 3,500 cP, about 50 cP to about 3,000 cP, about 50 cP to about 2,500 cP, about 50 cP to about 2,000 cP, about 50 cP to about 1,500 cP, about 50 cP to about 1,000 cP, about 50 cP to about 500 cP, about 50 cP to about 400 cP, about 50 cP to about 300 cP, about 50 cP to about 200 cP, about 50 cP to about 100 cP, about 100 cP to about 5,500 cP, about 100 cP to about 5,000 cP, about 100 cP to about 4,500 cP, about 100 cP to about 4,000 cP, about 100 cP to about 3,500 cP, about 100 cP to about 3,000 cP, about 100 cP to about 2,500 cP, about 100 cP to about 2,000 cP, about 100 cP to about 1,500 cP, about 100 cP to about 1,000 cP, about 100 cP to about 500 cP, about 100 cP to about 400 cP, about 100 cP to about 300 cP, about 100 cP to about 200 cP, about 200 cP to about 5,500 cP, about 200 cP to about 5,000 cP, about 200 cP to about 4,500 cP, about 200 cP to about 4,000 cP, about 200 cP to about 3,500 cP, about 200 cP to about 3,000 cP, about 200 cP to about 2,500 cP, about 200 cP to about 2,000 cP, about 200 cP to about 1,500 cP, about 200 cP to about 1,000 cP, about 200 cP to about 500 cP, about 200 cP to about 400 cP, about 200 cP to about 300 cP, about 300 cP to about 5,500 cP, about 300 cP to about 5,000 cP, about 300 cP to about 4,500 cP, about 300 cP to about 4,000 cP, about 300 cP to about 3,500 cP, about 300 cP to about 3,000 cP, about 300 cP to about 2,500 cP, about 300 cP to about 2,000 cP, about 300 cP to about 1,500 cP, about 300 cP to about 1,000 cP, about 300 cP to about 500 cP, about 300 cP to about 400 cP, about 400 cP to about 5,500 cP, about 400 cP to about 5,000 cP, about 400 cP to about 4,500 cP, about 400 cP to about 4,000 cP, about 400 cP to about 3,500 cP, about 400 cP to about 3,000 cP, about 400 cP to about 2,500 cP, about 400 cP to about 2,000 cP, about 400 cP to about 1,500 cP, about 400 cP to about 1,000 cP, about 400 cP to about 500 cP, about 500 cP to about 5,500 cP, about 500 cP to about 5,000 cP, about 500 cP to about 4,500 cP, about 500 cP to about 4,000 cP, about 500 cP to about 3,500 cP, about 500 cP to about 3,000 cP, about 500 cP to about 2,500 cP, about 500 cP to about 2,000 cP, about 500 cP to about 1,500 cP, about 500 cP to about 1,000 cP, about 1,000 cP to about 5,500 cP, about 1,000 cP to about 5,000 cP, about 1,000 cP to about 4,500 cP, about 1,000 cP to about 4,000 cP, about 1,000 cP to about 3,500 cP, about 1,000 cP to about 3,000 cP, about 1,000 cP to about 2,500 cP, about 1,000 cP to about 2,000 cP, about 1,000 cP to about 1,500 cP, about 1,500 cP to about 5,500 cP, about 1,500 cP to about 5,000 cP, about 1,500 cP to about 4,500 cP, about 1,500 cP to about 4,000 cP, about 1,500 cP to about 3,500 cP, about 1,500 cP to about 3,000 cP, about 1,500 cP to about 2,500 cP, about 1,500 cP to about 2,000 cP, about 2,000 cP to about 5,500 cP, about 2,000 cP to about 5,000 cP, about 2,000 cP to about 4,500 cP, about 2,000 cP to about 4,000 cP, about 2,000 cP to about 3,500 cP, about 2,000 cP to about 3,000 cP, about 2,000 cP to about 2,500 cP, about 2,500 cP to about 5,500 cP, about 2,500 cP to about 5,000 cP, about 2,500 cP to about 4,500 cP, about 2,500 cP to about 4,000 cP, about 2,500 cP to about 3,500 cP, about 2,500 cP to about 3,000 cP, about 3,000 cP to about 5,500 cP, about 3,000 cP to about 5,000 cP, about 3,000 cP to about 4,500 cP, about 3,000 cP to about 4,000 cP, about 3,000 cP to about 3,500 cP, about 3,500 cP to about 5,500 cP, about 3,500 cP to about 5,000 cP, about 3,500 cP to about 4,500 cP, about 3,500 cP to about 4,000 cP, about 4,000 cP to about 5,500 cP, about 4,000 cP to about 5,000 cP, about 4,000 cP to about 4,500 cP, about 4,500 cP to about 5,500 cP, about 4,500 cP to about 5,000 cP, or about 5,000 cP to about 5,500 cP.

In certain embodiments, during step (a), the cryopreservative mixture has a viscosity in the range of about 50 cP to about 4,000 cP, about 100 cP to about 2,000 cP, or about 100 cP to 1,000 cP. In certain embodiments, during step (a), the cryopreservative mixture has a viscosity in the range of about 100 cP to about 1,000 cP.

In certain embodiments, during step (a), the cryopreservative mixture has a viscosity of about 5 cP, about 10 cP, about 15 cP, about 20 cP, about 25 cP, about 30 cP, about 35 cP, about 40 cP, about 45 cP, about 50 cP, about 55 cP, about 60 cP, about 65 cP, about 70 cP, about 75 cP, about 80 cP, about 85 cP, about 90 cP, about 95 cP, about 100 cP, about 110 cP, about 120 cP, about 130 cP, about 140 cP, about 150 cP, about 160 cP, about 170 cP, about 180 cP, about 190 cP, about 200 cP, about 225 cP, about 250 cP, about 275 cP, about 300 cP, about 350 cP, about 400 cP, about 450 cP, about 500 cP, about 600 cP, about 700 cP, about 800 cP, about 900 cP, about 1,000 cP, about 1,250 cP, about 1,500 cP, about 2,000 cP, about 2,500 cP, about 3,000 cP, about 3,500 cP, about 4,000 cP, about 4,500 cP, about 5,000 cP, about 5,500 cP, about 6,000 cP.

In certain embodiments, during step (a), the cryopreservative mixture has an osmolality in the range of about 300 Osm/L to about 4,000 Osm/L, about 400 Osm/L to about 4,000 Osm/L, about 500 Osm/L to about 4,000 Osm/L, about 000 Osm/L to about 4,000 Osm/L, about 700 Osm/L to about 4,000 Osm/L, about 800 Osm/L to about 4,000 Osm/L, about 900 Osm/L to about 4,000 Osm/L, about 1,000 Osm/L to about 4,000 Osm/L, about 1,500 Osm/L to about 4,000 Osm/L, about 2,000 Osm/L to about 4,000 Osm/L, about 2,500 Osm/L to about 4,000 Osm/L, about 3,000 Osm/L to about 4,000 Osm/L, about 3,500 Osm/L to about 4,000 Osm/L, about 300 Osm/L to about 3,500 Osm/L, about 300 Osm/L to about 3,000 Osm/L, about 300 Osm/L to about 2,500 Osm/L, about 300 Osm/L to about 2,000 Osm/L, about 300 Osm/L to about 1,500 Osm/L, about 300 Osm/L to about 1,000 Osm/L, about 300 Osm L to about 900 Osm/L, about 300 Osm/L to about 800 Osm/L, about 300 Osm/L to about 700 Osm/L, about 300 Osm/L to about 600 Osm/L, about 300 Osm/L to about 500 Osm/L, about 300 Osm/L to about 400 Osm/L, about 400 Osm/L to about 3,500 Osm/L, about 400 Osm/L to about 3,000 Osm/L, about 400 Osm/L to about 2,500 Osm/L, about 400 Osm/L to about 2,500 Osm/L, about 400 Osm/L to about 2,000 Osm/L, about 400 Osm/L to about 2,000 Osm/L, about 400 Osm/L to about 1,500 Osm/L, about 400 Osm/L to about 1,000 Osm/L, about 400 Osm/L to about 900 Osm/L, about 400 Osm/L to about 800 Osm/L, about 400 Osm/L to about 700 Osm/L, about 400 Osm/L to about 600 Osm/L, about 400 Osm/L to about 500 Osm/L, about 500 Osm/L to about 3,500 Osm/L, about 500 Osm/L to about 3,000 Osm/L, about 500 Osm/L to about 2,500 Osm/L, about 500 Osm/L to about 2,000 Osm/L, about 500 Osm L to about 1,500 Osm/L, about 500 Osm/L to about 1,000 Osm/L, about 500 Osm/L to about 900 Osm/L, about 500 Osm/L to about 800 Osm/L, about 500 Osm/L to about 700 Osm/L, about 500 Osm/L to about 600 Osm/L, about 600 Osm/L to about 3,500 Osm/L, about 600 Osm/L to about 3,000 Osm/L, about 600 Osm/L to about 2,500 Osm/L, about 600 Osm/L to about 2,000 Osm/L, about 6010 Osm L to about 1,500 Osm/L, about 600 Osm/L to about 1,000 Osm/L, about 600 Osm/L to about 900 Osm/L, about 600 Osm/L to about 800 Osm/L, about 600 Osm/L to about 700 Osm/L, about 700 Osm/L to about 3,500 Osm/L, about 700 Osm/L to about 3,000 Osm/L, about 700 Osm/L to about 2,500 Osm/L, about 700 Osm/L to about 2,000 Osm/L, about 700 Osm/L to about 1,500 Osm/L, about 700 Osm/L to about 1,000 Osm/L, about 700 Osm/L to about 900 Osm/L, about 700 Osm/L to about 800 Osm/L, about 800 Osm/L to about 3,500 Osm/L, about 800 Osm/L to about 3,000 Osm/L, about 800 Osm/L to about 2.500 Osm/L, about 800 Osm/L to about 2,000 Osm/L, about 800 Osm/L to about 1,500 Osm/L, about 800 Osm/L to about 1,000 Osm/L, about 800 Osm/L to about 900 Osm/L, about 900 Osm/L to about 3,500 Osm/L, about 900 Osm/L to about 3,000 Osm/L, about 900 Osm/L to about 2,500 Osm/L, about 900 Osm/L to about 2,000 Osm/L, about 900 Osm/L to about 1,500 Osm/L, about 900 Osm L to about 1,000 Osm/L, about 1,000 Osm/L to about 3,500 Osm/L, about 1,000 Osm/L to about 3,000 Osm/L, about 1,000 Osm/L to about 2,500 Osm/L, about 1,000 Osm/L to about 2,000 Osm/L, about 1,000 Osm/L to about 1,500 Osm/L, about 1,500 Osm/L to about 3,500 Osm/L, about 1,500 Osm/L to about 3,000 Osm/L, about 1,500 Osm/L to about 2,500 Osm/L, about 1,500 Osm/L to about 2,000 Osm/L, about 2,000 Osm/L to about 3,500 Osm/L, about 2,000 Osm/L to about 3,000 Osm/L, about 2,000 Osm/L to about 2,500 Osm/L, about 2,500 Osm/L to about 3,500 Osm/L, about 2,500 Osm/L to about 3,000 Osm/L, or about 3,000 Osm/L to about 3,500 Osm/L.

In certain embodiments, during step (a), the cryopreservative mixture has an osmolality in the range of about 300 Osm/L to about 3,000 Osm/L, about 400 Osm/L to about 3,000 Osm/L, or about 500 Osm/L to about 3,000 Osm/L. In certain embodiments, during step (a), the cryopreservative mixture has an osmolality in the range of about 500 Osm/L to about 3,000 Osm/L.

In certain embodiments, during step (a), the cryopreservative mixture has an osmolality of about 300 Osm/L, about 350 Osm/L, about 400 Osm/L, about 450 Osm/L, about 500 Osm/L, about 600 Osm/L, about 700 Osm/L, about 800 Osm/L, about 900 Osm/L, about 1,000 Osm/L, about 1,250 Osm/L, about 1,500 Osm/L, about 1,750 Osm/L, about 2,000 Osm/L, about 2,250 Osm/L, about 2,500 Osm/L, about 2,750 Osm/L, about 3,000 Osm/L, about 3,500 Osm/L, or about 4,000 Osm/L.

In certain embodiments, during step (a), the cryopreservative mixture has a pH in the range of about 4 to about 8, about 4.5 to about 8, about 5 to about 8, about 5.5 to about 8, about 6 to about 8, about 6.5 to about 8, about 7 to about 8, about 7.5 to about 8, about 4 to about 7.5, about 4 to about 7, about 4 to about 6.5, about 4 to about 6, about 4 to about 5.5, about 4 to about 5, about 4 to about 4.5, about 4.5 to about 7.5, about 4.5 to about 7, about 4.5 to about 6.5, about 4.5 to about 6, about 4.5 to about 5.5, about 4.5 to about 5, about 5 to about 7.5, about 5 to about 7, about 5 to about 6.5, about 5 to about 6, about 5 to about 5.5, about 5.5 to about 7.5, about 5.5 to about 7, about 5.5 to about 6.5, about 5.5 to about 6, about 6 to about 7.5, about 6 to about 7, about 6 to about 6.5, about 6.5 to about 7.5, about 6.5 to about 7, or about 7 to about 7.5.

In certain embodiments, during step (a), the cryopreservative mixture has a pH in the range of about 4 to about 8, about 5 to about 8, about 4 to about 7.5, about 5 to about 7.5, or about 5.5 to about 7.5. In certain embodiments, during step (a), the cryopreservative composition has a pH in the range of about 5.5 to about 7.5.

In certain embodiments, during step (a), the cryopreservative mixture has a pH of about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, or about 8.

In certain embodiments, during step (a), the cryopreservative mixture has a redox potential in the range of −50 mV to −500 mV, −50 mV to −450 mV, −50 mV to −400 mV, −50 mV to −350 mV, −50 mV to −300 mV, −50 mV to −250 mV, −50 mV to −200 mV, −50 mV to −150 mV, −50 mV to −100 mV, −100 mV to −500 mV, −100 mV to −450 mV, −100 mV to −400 mV, −100 mV to −350 mV, −100 mV to −300 mV, −100 mV to −250 mV, −100 mV to −200 mV, −100 mV to −150 mV, −150 mV to −500 mV, −150 mV to −450 mV, −150 mV to −400 mV, −150 mV to −350 mV, 150 mV to −300 mV, −150 mV to −250 mV, −150 mV to −200 mV, −200 mV to −500 mV, −200 mV to −450 mV, −200 mV to −400 mV, −200 mV to −350 mV, −200 mV to −300 mV, −200 mV to 250 mV, −250 mV to −500 mV, −250 mV to −450 mV, −250 mV to −400 mV, −250 mV to −350 mV, −250 mV to −300 mV, −300 mV to −500 mV, −300 mV to −450 mV, −300 mV to −400 mV, 300 mV to −350 mV, −350 mV to −500 mV, −350 mV to −450 mV, −350 mV to −400 mV, −400 mV to −500 mV, −400 mV to −450 mV, or −450 mV to −500 mV. In certain embodiments, during step (a), the cryopreservative mixture has a redox potential in the range of −50 mV to −500 mV, When solvated in an aqueous solvent.

In certain embodiments, during step (a), the cryopreservative mixture has a redox potential of about −50 mV, about −00 mV, about −70 mV, about −80 mV, about −90 mV, about 100 mV, about −110 mV, about −120 mV, about −130 mV, about −140 mV, about −150 mV, about −160 mV, about −170 mV, about −180 mV, about −190 mV, about −200 mV, about −240 mV, about −260 mV, about −280 mV, about −300 mV, about −320 mV, about −340 mV, about −360 mV, about −380 mV, about −400 mV, about −420 mV, about −440 mV, about −460 mV, about −480 mV, or about −500 mV.

In certain embodiments, the cryopreservation method further comprises freezing the cryopreservative mixture prior to step (b). It is appreciated that the freezing process should be tailored for the organisms or group of organisms being cryopreserved. In certain embodiments, the cryopreservative mixture can contain a cryoprotectant and/or lyoprotectant. Depending upon the organisms desired, the cell compositions can be subjected to different freezing rates. The resulting mixture can then be tested to determine the appropriate freezing rate for the microorganism or group of microorganisms. It is understood that when a group of microorganisms are being frozen together, the freezing rate chosen may be a compromise between the optimal freezing rates for the individual organisms. In this situation, the freezing rate is chosen to optimize the viability of the various organisms being frozen together. In certain embodiments, the cryopreservative composition is held at a temperature of −35° C. or below for a period of at least 24 hours prior to step (b).

Exemplary cryoprotectants include, for example, acetamide, albumin, ammonium acetate, choline magnesium chloride, sodium bromide, diethyl glycol, dimethylacetamide, dimethylsulfoxide, ethanol, erythritol, glycerol, glucose, formamide, glycerophosphate, proline, pyridine-N-oxide, propylene glycol, ribose, serine, sodium chloride, sodium iodide, sodium sulfate, sorbitol, sucrose, trehalose, triethylene glycol, trimethylamine, valine, xylose. Exemplary lyoprotectants include, for example, alginates, maltodextrin, oligofructose, etc. It is understood that certain cryoprotectants can also act as lyoprotectants.

During step (b), the freeze drying conditions can be selected to optimize the viability of the organism or group of different organisms in the cryopreservative mixture.

In certain embodiments, during step (b), freeze drying the cryopreservative composition comprises: (i) equilibrating the cryopreservative composition at a first temperature of about −35° C. for about 2 hours; (ii) increasing the temperature of the cryopreservative composition to a second temperature of about −25° C. to −30° C.; (iii) holding the cryopreservative composition at the second temperature and a first pressure of less than or equal to 55 mTorr for about 20-72 hours; (iv) increasing the temperature of the cryopreservative composition to a third temperature of about 4 to 20° C.; and (v) holding the cryopreservative composition at the third temperature and a second pressure of about 100 mTorr for about 20-48 hours or until the water activity of the cryopreservative composition is less than about 0.15, thereby producing the cryopreserved composition.

In certain embodiments, in step (ii), the temperature of the cryopreservative composition is increased to the second temperature of about −25° C. at a warming rate of about 0.01° C./min. In certain embodiments, in step (iv), the temperature of the cryopreservative composition is increased to the third temperature of about 20° C. at a warming rate of about 0.1° C./min.

In certain embodiments, during step (b), the cryopreserved composition has a water content of less than about 0.5% (w/w), less than about 1% (w/w), less than about 1.5% (w/w), less than about 2% (w/w), less than about 2.5% (w/w), less than about 3% (w/w), less than about 3.5% (w/w), less than about 4% (w/w), less than about 4.5% (w/w), or less than about 50% (w/w).

In certain embodiments, during step (b), the cryopreserved composition has a water content in the range of about 1% (w/w) to about 5% (w/w), about 1.5% (w/w) to about 5% (w/w), about 2% (w/w) to about 5% (w/w), about 2.5% (w/w) to about 5% (w/w), about 3% (w/w) to about 5% (w/w), about 3.5% (w/w) to about 5% (w/w), about 4% (w/w) to about 5% (w/w), about 4.5% (w/w) to about 5% (w/w), about 1% (w/w) to about 4.5% (w/w), about 1% (w/w) to about 4% (w/w), about 1% (w/w) to about 3.5% (w/w), about 1% (w/w) to about 3% (w/w), about 1% (w/w) to about 2.5% (w/w), about 1% (w/w) to about 2% (w/w), about 1% (w/w) to about 1.S % (w/w), about 1.5% (w/w) to about 4.5% (w/w), about 1.5% (w/w) to about 4% (w/w), about 1.5% (w/w) to about 3.5% (w/w), about 1.5% (w/w) to about 3% (w/w), about 1.5% (w/w) to about 2.5% (w/w), about 1.5% (w/w) to about 2% (w/w), about 2% (w/w) to about 4.5% (w/w), about 2% (w/w) to about 4% (w/w), about 2% (w/w) to about 3.5% (w/w), about 2% (w/w) to about 3% (w/w), about 2% (w/w) to about 2.5% (w/w), about 2.5% (w/w) to about 4.5% (w/w), about 2.5% (w/w) to about 4% (w/w), about 2.5% (w/w) to about 3.5% (w/w), about 2.5% (w/w) to about 3% (w/w), about 3% (w/w) to about 4.5% (w/w), about 3% (w/w) to about 4% (w/w), about 3% (w/w) to about 3.5% (w/w), about 3.5% (w/w) to about 4.5% (w/w), about 3.5% (w/w) to about 4% (w/w), or about 4% (w/w) to about 4.5% (w/w). In certain embodiments, in step (b), the cryopreserved composition has a water content in the range of about 2% (w/w) to about 4% (w/w) or about 2% (w/w) to about 3% (w/w). In certain embodiments, the composition has a water content in the range of about 1.5% (w/w) to about 4% (w/w).

In certain embodiments, during step (b), the cryopreserved composition has a water content of about 0.5% (w/w), about 1% (w/w), about 1.5% (w/w), about 2% (w/w), about 2.5% (w/w), about 3% (w/w), about 3.5% (w/w), about 4% (w/w), about 4.5% (w/w), or about 5% (w/w).

In various embodiments, the method further comprises packaging the cryopreserved composition into a capsule, tablet or caplet as described above, where the capsule, tablet or caplet may be coated with a delayed release coating.

In various embodiments, the method optionally further comprises the various steps including: (c) sieving the cryopreserved composition through a #20 US Mesh (0.85 mm) screen; (d) blending the cryopreserved composition; and (e) packaging the cryopreserved composition in a capsule, tablet or caplet.

In various embodiments, the method optionally further comprises the steps of (c) sieving the cryopreserved composition through a #20 US Mesh (0.85 mm) screen; (d) blending the cryopreserved composition with one or more pharmaceutically acceptable excipients to produce a pharmaceutical composition described herein; and (e) packaging the pharmaceutical composition in a capsule, tablet or caplet.

### 5. Methods of Use and Treatment

The compositions described herein can be used in methods of modulating the gastrointestinal microbiome of a subject in need thereof. The method generally comprises administering to the subject an effective amount of a preserved composition, e.g., cryopreserved composition, described herein. e.g., in the form of a pharmaceutical composition or a food product/probiotic. Depending upon the certain circumstances, the method comprises administering to the subject an effective amount of a pharmaceutical composition described herein or the method comprises administering to the subject an effective amount of a food product or probiotic described herein.

The preserved (e.g. cryopreserved) compositions described herein can be used to treat a variety of conditions, diseases or disorders, such as gastrointestinal condition, disease, or disorder. The gastrointestinal condition, disease, or disorder can be a condition, disease, or disorder of the small intestine, e.g., Crohn's disease or ulcerative colitis.

### Examples

In order that the disclosure described herein may be more fully understood, the following examples are set forth. The process and analytical examples described in this application are offered to illustrate the compositions and methods provided herein and are not to be construed in any way as limiting their scope.

### Example 1: Assay Protocols

### Viable Cell Count/Total Cell Count/Non-viable Cell Count:

The assays used herein employed Molecular Probes' LIVE/DEAD® BacLight™ Bacterial Viability Kits, which is a two-color fluorescence assay of bacteria viability. The kits use a mixture of SYTO® 9 green-fluorescent nucleic acid stain and the red-fluorescent nucleic acid stain, propidium iodide. The stains differ both in their spectral characteristics and in their ability to penetrate healthy bacterial cells. When used alone, the SYTO® 9 stain generally labels all bacteria in a population, namely those with intact membranes and those with damaged membranes. In contrast, propidium iodide penetrates only bacteria with damaged membranes, causing a reduction in the SYTO® 9 stain fluorescence when both dyes are present. Thus, with an appropriate mixture of the SYTO® 9 and propidium iodide stains, bacteria with intact cell membranes stain fluorescent green, whereas bacteria with damaged membranes stain fluorescent red. The excitation/emission maxima for these dyes are about 480/500 nm for SYTO® 9 stain and 490/635 nm for propidium iodide. The background remains virtually non-fluorescent. Fluorescence intensity is measured using a Synergy HT Multi-Detection Microplate Reader (BioTek®) or the like, using a 488/20 nm excitation filter (for both SYTO® 9 and propidium iodide) and a 528/20 nm (SYTO9 emission wavelength) and 645/40 nm (propidium iodide emission wavelength) emission filter.

In order implement the method, equal volumes of Component A (SYTO® 9 dye, 3.34 mM) and Component B (Propidium Iodide, 20 mM) of the kit were combined and mixed thoroughly and then 3 μL of dye mixture for every mL of bacterial suspension (8-10 mg/mL) was added. The dye mixture and bacterial suspension were then mixed thoroughly and incubated at room temperature in the dark for 15 minutes. Then 5 μL of the stained bacterial suspension was spread on a microscope slide with a coverslip and observed using a fluorescence microscope equipped with a filter set in accordance with the manufacturer's instruction.

The appearance of the organisms was determined in accordance with United States Pharmacopeia (USP)<631>(Color and Achromicity).

### Water Content

Total water (free and bound) in the lyophilized powder was determined using the Karl Fischer method, as per USP<921>(Water Determination).

### Water Activity

The water activity in the cryopreserved powders described below was tested using a method based on the method of Water Activity, AOAC International Official Method 978.18 (Official Methods o/Analysis of AOAC International, 17^{th} edition, AOAC International) per the guidance given in USP<1112>, Application of Water Activity Determination to Nonsterile Pharmaceutical Products.

### Total Aerobic Microbial Count

Total Aerobic Microbial Count (TAMC) was used to determine the total number of aerobic bacteria present in the rehydrated cryopreserved sample at mesophilic temperatures (30 37° C.) as per USP<61>(Microbiological Examination of Nonsterile Products: Microbial Enumeration Tests).

### Total Combined Yeast and Mold Count

Total Combined Yeast and Mold Count (TYMC) was tested as per USP<62>(Microbiological Examination of Nonsterile Products: Tests for Specified Microorganisms).

### Optical Density (OD at 600 nm) of Cryopreserved Samples

Using a 1 cc syringe and 21-gauge needle, a 1 mL cryopreserved sample was taken from a growing culture vial/bottle. Prior to sampling, the contents of the vial were mixed thoroughly and the syringe was adequately flushed with CO₂. The 1 mL sample was then dispensed into a micro-centrifuge tube and optical density measurements were recorded at 600 nm (OD₆₀₀). If the OD₆₀₀ was less than 0.5 the cell suspension was directly dispensed into a cuvette and the optical density measured at 600 nm. If the OD₆₀₀ was greater than 0.5, serial dilutions were prepared in distilled H₂O until the OD₆₀₀ was below 0.5, and the measurement recorded.

### pH of Cryopreserved Samples

The pH of the cryopreserved samples was tested using USP<791>(the standardized test method for pH measurements used in the pharmaceutical, food and beverage, patient care, and dietary supplement industries).

### Osmolality of Cryopreserved Samples

The osmolality of the cryopreserved samples was tested using USP<785>(osmolality and osmolarity).

### Viscosity of Cryopreserved Samples

The viscosity of the cryopreserved samples was tested using USP<911>(viscosity).

### Thermal Characterization of Cryopreserved Samples

### (i) Differential Scanning Calorimetry (DSC) Method

The DSC instrument used for the thermal characterization (e.g., determination of the glass transition temperature (Tg′)) of the cryopreserved samples described in the Examples herein was a TA Instruments Discovery DSC25 modulated DSC with an attached refrigerated cooling system.

Prior to characterizing the cryopreserved samples, the calibration of the DSC instrument was checked with a 1% sodium chloride solution. The sodium chloride calibration sample was determined to have an onset of melt at −21.71° C. (theoretical value=−21.5° C.).

In preparation for freezing for DSC analysis, using a pipette, an 18 μL aliquot of the liquid (e.g., a sodium chloride calibration sample or a liquid sample of a cryopreservative solution) to be analyzed was pipetted into an aluminum DSC sample pan and hermetically sealed.

DSC method: the sample and reference pan (an empty aluminum pan) were loaded into the DSC at 40° C. and were then cooled to −90° C. at 10° C./min. The pans were then held 2 minutes at −90° C. and subsequently heated to 30° C. at 10° C./min.

### (ii) Freeze-Dry Microscopy Method

The freeze-dry microscopy instrument includes a Nikon Eclipse LV 100Npol polarized light microscope with a Linkam FDCS 196 thermal stage, a T96 controller, a LNP96 liquid nitrogen pump, and an Edwards E2M 1.5 vacuum pump.

Method: a 5 μL aliquot of sample (e.g., a liquid sample of a cryopreservative solution) was placed on a glass slide which had been placed on the thermal stage after applying a small drop of silicone oil. A coverslip was placed over the sample and the chamber was sealed. The sample was cooled to −45° C. at 10° C./min and held for 2 minutes and a micrograph was captured. The vacuum was initiated, and the sample was held at a fixed temperature of −45° C. until the sublimation front came into view. Another micrograph was then captured. The sample was then warmed at 10° C./minute to −10° C. Micrographs were collected as the sample went through the different thermal transitions (e.g., the collapse temperature (Tc)).

### Example 2: Cell Culture and Harvest

This example describes a procedure used to cultivate and harvest each of the organisms described herein.

A cell bank was thawed in a room temperature water bath for approximately 15 minutes. A filtered, blunt needle was prepared with anaerobic gas, flowing into a 50 mL Falcon tube. A syringe was flushed with the anaerobic gas (e.g. N₂, CO₂, Argon) in the Falcon tube to remove oxygen. With a needle, the amount of thawed bank media required for the desired percent inoculation was introduced into a vial of anaerobic medium or bottle.

The media components were weighed out and water added to a specified volume and mixed until dissolved, then autoclaved at 121° C. with a 60-minute liquid cycle or sterile filtered into a sterilized vessel. The medium was sparged with 100% CO₂ or N₂ at 2 liters per minute with a stir rate of 150 rpm (6 blade Rushton impeller). The set-point was set to 37° C. and sparging continued at 2 liters per minute until the redox potential was sufficiently negative, namely within the range from about −50 mV to about −500 mV. The gas was switched from sparging the media to purging the headspace with anaerobic gas at 1 liter per minute once the redox potential meets specified criteria and then maintained at an overflow at 1 liter per minute.

When a 2 L anaerobic bottle first reached a specified OD₆₀₀, the culture was ready to transfer to the fermenter. Then, 100 mL of culture media was withdrawn from the bottle and added to the fermenter via an inoculation port. The fermentation was allowed to continue for a specified amount of time.

Harvesting was performed via a Repligen Hollow Fiber PES 750 kDa membrane where 10 L is concentrated to approximately 400-600 mL. The retentate was collected and filled into centrifuge bottles within the anaerobic chamber. The retentate was centrifuged for 30 minutes at 10,000 rpm, and cell paste was collected within the anaerobic chamber and further processed.

### Example 3: Preparation of Preservation Compositions for Anaerobic Microorganisms

For each excipient blend set forth in Table 1 below, all dry components (e.g., raftilose, maltodextrin, sodium alginate, and trehalose and/or sucrose) were dry blended until the powdered mixture appeared homogeneous. The powdered mixtures were then slowly added to deionized (Dl) water in a second container and stirred until all components dissolved. Thereafter, the resulting solution was placed in an anaerobic chamber and continually stirred for 60 minutes. The pH of the excipient blend was adjusted using either NaOH or HCl, while the pH was monitored using a pH meter, until the pH reached a level optimal to the organism/organisms. The excipient blend was then sterile filtered into a sterile container using a 0.45 micron filter.

Within an anaerobic chamber, frozen cell paste (cell concentrate) or fresh cell paste was placed into a sterile container. The cell paste was mixed at a 1:1, 1:2, or 1:3 ratio with an anaerobic buffer, spent media or fresh media, as desired. The mixture was mixed for 20-30 minutes, and the resulting blend was poured into lyophilization trays to a defined fill depth. The lyophilization trays were placed into a plastic bag and into a laminate foil pouch and finally heat sealed, all within an anaerobic chamber so as maintain a reduced redox potential within the range of −50 to −500 mV. The sealed trays were introduced into a freezer or lyophilization shelf to freeze and were permitted to freeze for at least 24 hours prior to initiating freeze drying.

TABLE 1
Cryopreservation Compositions and Properties

| | Blend Number | | |
|---|---|---|---|
| | ACP-001 | ACP-002 | ACP-003 |
| % Raftilose | 0.5 | 0.5 | 0.5 |
| % Maltodextrin | 4.5 | 3 | 3 |
| % Alginate | 0.4 | 0.4 | 0.4 |
| % Trehalose | 10 | 10 | 5 |
| % Cell Paste | 50 | 33.3 | 50 |
| % DI H₂O | 34.6 | 52.8 | 36.1 |
| % Sucrose | 0 | 0 | 5 |
| pH | 6.5-7.5 | | |
| Osmolality | >2000 | 564 | >2000 |
| Viscosity | 350 | 112 | 880 |
| Tg′ (° C.) | −28 | −28 | −33 |
| Tc (° C.) | −23 | −23 | −25 |

### Example 4: Preparation of Compositions for Cryopreserving Anaerobic Microorganisms and Potency Testing

The cryopreservation compositions, as prepared in Example 3, were then lyophilized as follows:

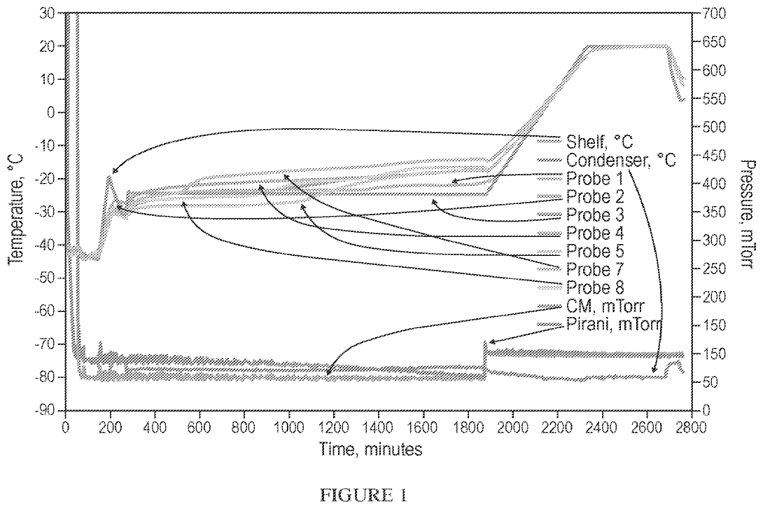

Prior to lyophilization, the frozen composition was frozen at temperatures between −35° C. to −150° C. and then placed into the lyophilizer (Virtis, Genesis SQ Super XL70) and equilibrated at −35° C. A three-step ramp cycle was used to lyophilize the cryopreservation compositions, with the lyophilzation process ending when the water activity was <0.15 during secondary drying. The lyophilization parameters are listed in Table 2 and the temperature/pressure profiles used during the lyophilization cycle are shown in FIG. 1. The lyophilization cycle is approximately 90 hours in total. The lyophilized cake was then stored at −20° C. until further processing.

The glass transition temperature of the lyophilized cake. Tg′, was determined to be −24° C.

TABLE 2
Lyophilization Cycle Parameters

| Lyophilization Parameter | Set point and time intervals |
|---|---|
| Product loading temperature | −35° C. or below |
| Product freeze-back time | hold for 120 min |
| First step | Ramp from −35° C. to −30° C. (60 |
| | min) and hold for 48 hours at 55 |
| | mTorr or below |
| Second step | Ramp from −25° C. to 20° C. at |
| | 0.1° C./min |
| Third step | Hold at 20° C. and 100 mTorr for |
| | 31.5 hours or until Aw is below |
| | 0.15 |
| Unload shelf temperature | 20° C. |

The lyophilized cake was then sieved through a #20 US Mesh (0.85 mm) screen followed by mixing within the sieve pan for a duration of 2 minutes. Sieving, blending and packaging were all carried out in an atmosphere with ≤ 15% relative humidity (RH) in a nitrogen glove box or anaerobic chamber. The resulting material (Drug Substance) was portioned and packaged in polyethylene bags and placed inside foil-laminated pouches along with a desiccant pouch, then sealed. The sealed pouches with the Drug Substance were stored at less than −20° C. Samples were pulled and tested for % viability.

Viability results are presented in Table 3 for three cryopreservation mixes (ACP-001. ACP-002 and ACP-003) as compared to two commercial blend mixtures (CP-001 and CP-002) (OPS Diagnostics Cat. No. LR2X-500-02 and MFDB-500-06).

TABLE 3
Viability of 30 representative families/genus of bacteria after being freeze dried
in blends ACP-001, ACP-002, ACP-003 and two commercial blends (CP-001 and CP-002)

| | | ACP- | ACP- | ACP- | CP- | CP- |
|---|---|---|---|---|---|---|
| Family | Genus | 001 | 002 | 003 | 001\hat{ } | 002\hat{ }\hat{ } |
| Akkermansiaceae | Akkermansia | 76.80% | 71.80% | 60% | ND | ND |
| Bacillaceae | Bacillus | 52% | 68% | 63% | ND | ND |
| Bacteroidaceae | Bacteroides | 50% | 38% | 45% | 21% | 13% |
| Bifidobacteriaceae | Bifidobacterium | 85% | 100% | 95% | ND | ND |
| Christensenellaceae | Christensenella | 82% | 100% | 100% | ND | ND |
| Clostridiaceae | Butyricoccus | 7% | 16% | 8% | ND | ND |
| Coriobacteriaceae | Slackia | 43% | 59% | 74% | ND | ND |
| Eubacteriaceae | Eubacterium | 41.70% | 44.60% | 51.40% | 24% | 9% |
| Lachnospiraceae | Blautia | 86% | 72% | 93% | ND | ND |
| Lachnospiraceae | Dorea | 42% | 45% | 57% | ND | ND |
| Lachnospiraceae | Anaerostipes | 45.88% | 73.22% | 38.05% | ND | ND |
| Lachnospiraceae | Lachnoclostridium | 11.95% | 28.40% | 43.94% | 2.29% | 1.35% |
| Lachnospiraceae | Lachnospiraceae | 18% | 13% | 15% | ND | ND |
| Lachnospiraceae | Roseburia | 73% | 33% | 34% | ND | ND |
| Lactobacilloceae | Lactobacillus | 54% | 67% | 81% | 20% | ND |
| Leuconostocaceae | Leuconostoc | 90% | 91% | 94% | 10% | ND |
| Leuconostocaceae | Weisella | 78% | 71% | 62% | 24% | ND |
| Odoribacteraceae | Odorbacter | 52% | 42% | 35% | ND | ND |
| Oxalobacteraceae | Oxalobacter | 40% | 52% | 44% | 8% | 4% |
| Oxalobacteraceae | Janthinobacterium | 98% | 98% | 98% | ND | ND |
| Porphyromonadaceae | Barnesiella | 80% | 75% | 81% | ND | ND |
| Rikenellaceae | Alistipes | 44% | 67% | 37% | 12% | 15% |
| Ruminococcaceae | Ruminococcus | 60% | 50% | 41% | ND | ND |
| Ruminococcaceae | Faecalibacterium | 44% | 34% | 48% | ND | ND |
| Streptococcaceae | Streptococcus | 69% | 84% | 73% | 15% | ND |
| | Senegalmassilia | 95% | 100% | 100% | ND | ND |
| Enterobacteriaceae | Escherichia | 93% | 94% | 97% | ND | ND |
| Eggerthellaceae | Slackia | 39% | 55% | 47% | ND | ND |
| Clostridiaceae | Clostridium | 99% | 98% | 100% | ND | ND |
| Prevotellaceae | Prevotella | 37% | 44% | 23% | ND | ND |

\hat{ }OPS Diagnostics Catalog No. LR2X-500-02
\hat{ }\hat{ }OPS Diagnostics Catalog No. MFDB 500-06
ND: Not Determined

The results from Table 3 indicate that, for the various bacterial families and genus' examined, the resulting potency (viability) for the three ACP blends is better than that achieved using the two commercially available blends (OPS Diagnostics Cat. No. LR2X-500-02 and MFDB-500-06).

### Example 5: Stability of Cryopreserved Compositions

### Freeze Thaw Stability

Each freeze-thaw cycle was executed by removing the sample in its packaging from storage at −20±5° C. and placing it at ambient room temperature (22° C.) for at least 6 hours. After thawing, samples were then returned to storage at −20±5° C. for 12-24 hours. Samples were staggered through up to 4 freeze-thaw cycles, in order to ensure that all samples were analyzed at the same time.

The results are summarized in Table 4.

TABLE 4
Drug Substance Appearance, Water Content, and Viability Testing for Freeze Thaw Stability

| | Thaw 1 | | | Thaw 2 | | | Thaw 3 | | | Thaw 4 | | |
|---|---|---|---|---|---|---|---|---|---|---|---|---|
| Sample | A* | M** | V*** | A* | M** | V*** | A* | M** | V*** | A* | M** | V*** |
| Bacteroidaceae | P | 5.64 | 69.2 | P | 5.24 | 54.6 | P | 5.19 | 61.7 | P | 5.21 | 64.2 |
| Lachnospiraceae | P | 4.99 | 24.5 | P | 4.79 | 19.6 | P | 4.82 | 29.5 | P | 4.91 | 31.3 |
| Ruminococcaceae | P | 4.49 | 16.5 | P | 4.41 | 21.4 | P | 4.34 | 23.6 | P | 4.29 | 13.2 |
| Porphyromonadaceae | P | 5.33 | 34.6 | P | 5.08 | 35.7 | P | 5.10 | 29.5 | P | 5.07 | 40.6 |

*Appearance testing result: P = free-flowing powder, C = cake
**Moisture testing result, reported in % wt/wt
***Viability testing result, reported in % viability

The results of this study indicate that the Drug Substance (all four bacterial families tested) is stable for at least four freeze-thaw cycles from recommended storage temperature (−20±5° C.) to room temperature (22° C.).

### Freeze Cycle Stability

Each temperature cycle was executed by removing the sample in its packaging from storage at −20±5° C. and placing it on dry ice for 2 hours. After equilibrating at −80° C., samples were then returned to storage at −20±5° C. for 2 hours. Samples were staggered through up to 4 freeze-thaw cycles, in order to ensure that all samples were analyzed at the same time.

The results are summarized in Table 5.

TABLE 5
Drug Substance Appearance, Water Content, and Viability Testing for Freeze Cycle Stability

| Microorganism | Cycle 1 | | | Cycle 2 | | | Cycle 3 | | | Cycle 4 | | |
|---|---|---|---|---|---|---|---|---|---|---|---|---|
| Family | A* | M** | V*** | A* | M** | V*** | A* | M** | V*** | A* | M** | V*** |
| Bacteroidaceae | P | 5.64 | 69.2 | P | 5.29 | 73.45 | P | 5.25 | 65.47 | P | 5.26 | 59.87 |
| Lachnospiraceae | P | 4.99 | 24.5 | P | 4.68 | 21.38 | P | 4.64 | 25.67 | P | 4.66 | 20.24 |
| Ruminococcaceae | P | 4.49 | 16.5 | P | 4.37 | 22.78 | P | 4.33 | 19.85 | P | 4.31 | 17.89 |
| Porphyromonadaceae | P | 5.33 | 34.6 | P | 5.19 | 37.35 | P | 5.22 | 33.78 | P | 5.17 | 36.89 |

*Appearance testing result: P = free-flowing powder, C = cake
**Moisture testing result, reported in % wt/wt
***Viability testing result, reported in % viability

The results of this study indicate that Drug Substance (all four bacterial families tested) was stable at low temperature exposure at −80° C. as well as at least four temperature cycles between recommended storage temperature (−20±5° C.) and lower temperature (−80° C.).

### Long-Term Stability

A long-term stability study of the cryopreserved compositions prepared in Example 4 was conducted per storage condition (controlled versus accelerated), using the analytical methods provided in Table 6.

The stability studies were conducted according to ICH Q1 and ICH Q5 guidelines.

TABLE 6
Stability Study Analytical Methods for Cryopreserved Compositions at −20°
C. (Controlled Conditions), +5° C., +25° C. and >+28° C. (Excursion Condition)

| | Time point (months) | | | | | | | |
|---|---|---|---|---|---|---|---|---|
| | 0 | 1 | 3 | 6 | 9 | 12 | 18 | 24 |
| Appearance | Release | B, C, D, E | B, C, D, E | B, C, D, E | B, C, D | B, C, D | B, C, D | B, C, D |
| Identity (colony | testing | B, C, D, E | B, C, D, E | B, C, D, E | B, C, D | B, C, D | B, C, D | B, C, D |
| morphology) | | | | | | | | |
| Potency (CFU/g) | | B, C, D, E | B, C, D, E | B, C, D, E | B, C, D | B, C, D | B, C, D | B, C, D |
| Karl Fisher | | B, C, D, E | B, C, D, E | B, C, D, E | B, C, D | B, C, D | B, C, D | B, C, D |
| Total cell count | | B, C, D, E | B, C, D, E | B, C, D, E | B, C, D | B, C, D | B, C, D | B, C, D |
| Viable cell count | | B, C, D, E | B, C, D, E | B, C, D, E | B, C, D | B, C, D | B, C, D | B, C, D |
| (live/dead) | | | | | | | | |
| Growth recovery¹ | | B, C, D, E | B, C, D, E | B, C, D, E | B, C, D | B, C, D | B, C, D | B, C, D |
| Bioburden | | B, C, D, E | B, C, D, E | B, C, D, E | B, C, D | B, C, D | B, C, D | B, C, D |

Conditions: B −20 ± 5° C.; C +5 ± 3° C.; D +25 ± 3° C.; E >+28° C.

For clarity, only potency data (CFU/g of Drug Substance Powder) are presented for materials stored at 2-8° C. and 25° C. In addition, batches of material were included into a stability program that had less than 5% or more than 5% total moisture to determine the impact of moisture content on stability.

Results are shown in Tables 7-9.

Table 7 shows the stability at 2-8° C. (CFU/g in Log Scale) where the Drug Substance Powder has a total moisture content below 5% (w/w).

TABLE 7

| | Month | | | | | | | |
|---|---|---|---|---|---|---|---|---|
| | 0 | 1 | 3 | 6 | 9 | 12 | 18 | 24 |
| Lactobacillaceae | 10.59 | 10.62 | 10.57 | 10.51 | 10.57 | 10.59 | 10.52 | 10.61 |
| Oxalobacteraceae | 11.12 | 11.00 | 10.74 | 11.00 | 10.81 | 10.95 | 11.00 | 10.99 |
| Christensenellaceae | 10.00 | 10.30 | 10.20 | 10.40 | 10.30 | 10.15 | 10.20 | 10.24 |
| Bacteroidaceae | 11.35 | 11.30 | 11.39 | 11.43 | 11.24 | 11.34 | 11.23 | 11.40 |
| Lachnospiraceae | 10.97 | 11.10 | 10.94 | 10.91 | 11.14 | 11.13 | 10.95 | 10.99 |
| Ruminococcaceae | 10.40 | 10.30 | 10.35 | 10.24 | 10.45 | 10.42 | 10.25 | 10.33 |
| Clostridiaceae | 11.20 | 11.25 | 11.17 | 11.14 | 11.19 | 11.10 | 11.19 | 11.15 |

Table 8 shows the stability at 25° C. (CFU/g in Log Scale) where the Drug Substance Powder has a total moisture content below 5% (w/w).

TABLE 8

| | Month | | | | | | | |
|---|---|---|---|---|---|---|---|---|
| | 0 | 1 | 3 | 6 | 9 | 12 | 18 | 24 |
| Lactobacillaceae | 10.59 | 10.43 | 10.41 | 10.39 | 10.40 | 10.38 | 10.37 | 10.38 |
| Oxalobacteraceae | 11.12 | 11.00 | 10.75 | 10.81 | 10.91 | 10.93 | 10.94 | 10.87 |
| Christensenellaceae | 10.00 | 10.20 | 10.10 | 10.20 | 10.20 | 10.35 | 10.00 | 10.00 |
| Bacteroidaceae | 11.35 | 11.31 | 11.28 | 11.27 | 11.25 | 11.27 | 11.24 | 11.21 |
| Lachnospiraceae | 10.97 | 10.95 | 10.93 | 10.87 | 10.90 | 11.00 | 10.89 | 10.85 |
| Ruminococcaceae | 10.40 | 10.35 | 10.32 | 10.30 | 10.31 | 10.28 | 10.29 | 10.32 |
| Clostridiaceae | 11.20 | 11.25 | 11.22 | 11.19 | 11.14 | 11.15 | 11.20 | 11.14 |

Table 9 shows the stability at 25° C. (CFU/g in Log Scale) where the Drug Substance Powder has a total moisture content greater than 5%, but less than 8%.

TABLE 9

| | Month | | | | | | | |
|---|---|---|---|---|---|---|---|---|
| | 0 | 1 | 3 | 6 | 9 | 12 | 18 | 24 |
| Lactobacillaceae | 10.59 | 10.40 | 10.00 | 9.90 | 9.40 | 9.10 | 8.20 | 7.40 |
| Oxalobacteraceae | 11.12 | 10.87 | 10.54 | 10.30 | 10.10 | 9.70 | 9.20 | 8.90 |
| Bacteroidaceae | 11.35 | 11.22 | 11.14 | 10.87 | 10.70 | 10.30 | 10.10 | 9.80 |
| Lachnospiraceae | 10.97 | 10.60 | 10.40 | 10.10 | 9.97 | 9.60 | 9.40 | 9.10 |

The results in Tables 7-9 show that maintaining the moisture content below 5% is important for long-term stability. All of the organisms examined that were stored with a moisture content at or below 5% (w/w) were stable for 24 months when stored at both 2-8° C. and 25° C.

### Example 6: Characterization of Cryopreserved Compositions

### Drug Substance Air Exposure

Evaluation of the oxygen sensitivity of the cryopreserved compositions prepared in Example 4 was carried out by placing the samples in a sealed polyethylene bag (sealed in atmospheric air) at a temperature of 22° C. and a relative humidity of 19% via the use of a saturated 40% NaOH solution for up to 7 days. Sample exposures were staggered through the time points to ensure that all samples were analyzed at the same time.

The Drug Substance Appearance, Water Content, and Viability Testing were measured as a function of time in the presence of air. The results of this study are summarized in Table 10.

TABLE 10

| Microorganism | Day 0 | | | Day 1 | | | Day 3 | | | Day 7 | | |
|---|---|---|---|---|---|---|---|---|---|---|---|---|
| Family | A* | M** | V*** | A* | M** | V*** | A* | M** | V*** | A* | M** | V*** |
| Bacteroidaceae | P | 5.64 | 69.2 | P | 5.54 | 74.5 | P | 5.66 | 70.9 | P | 5.69 | 65.5 |
| Lachnospiraceae | P | 4.99 | 24.5 | P | 5.03 | 26.6 | P | 5.05 | 31.0 | P | 5.06 | 26.1 |
| Ruminococcaceae | P | 4.49 | 16.5 | P | 4.56 | 18.9 | P | 4.58 | 20.5 | P | 4.58 | 15.4 |
| Porphyromonadacede | P | 5.33 | 34.6 | P | 5.32 | 38.8 | P | 5.35 | 32.5 | P | 5.37 | 30.0 |

*Appearance testing result: P = free-flowing powder, C = cake
**Moisture testing result, reported in % wt/wt
***Viability testing result, reported in % viability

The results of this study indicate that Drug Substance was stable when stored at 22° C. and 19% relative humidity in the presence of atmospheric oxygen levels (−20.95%) over the course of up to 7 days for all four bacterial families examined. When stored in a dry dormant state, the organisms are less sensitive to oxygen exposure.

### Drug Substance Moisture Sorption Studies

The characteristics and impact of moisture uptake were evaluated under conditions described in Table 11. The relative humidity conditions were maintained via the use of saturated salt solutions and confirmed using an Acurite digital temperature/humidity meter to which the various samples were exposed.

TABLE 11
Summary of Conditions for Moisture Uptake Study

| | | Relative | NaOH Salt |
|---|---|---|---|
| Condition | Temp. (° C.) | Humidity (%) | Solution |
| 1 | 22 | 19 | 40.0% |
| 2 | 22 | 30 | 35.29% |
| 3 | 22 | 39 | 31.58% |
| 4 | 22 | 48 | 28.15% |
| 5 | 22 | 59 | 24.66% |
| 6 | 22 | Control* | NA |
| 7 | Control** | Control** | NA |

*The control samples (Condition 6) were stored in sealed packaging and not exposed to relative humidity.
**The control samples (Condition 7) were stored in sealed packaging and not exposed to relative humidity or elevated temperature. These samples were stored at −20° C.

The samples were exposed to the conditions for periods of 2, 4, 8, and 24 hours, in addition to initial testing before exposure.

The results are summarized in Tables 12-15, where the appearance, water content, and viability of the organisms were tested.

TABLE 12
Bacteroidaceae

| | T = initial | | | 2 Hr | | | 4 Hr | | | 8 Hr | | | 24 Hr | | |
|---|---|---|---|---|---|---|---|---|---|---|---|---|---|---|---|
| Condition | A* | M** | V*** | A* | M** | V*** | A* | M** | V*** | A* | M** | V*** | A* | M** | V*** |
| 1 | F | 5.6 | 69.2 | F | 5.7 | 72.4 | F | 5.7 | 64.5 | F | 5.7 | 75.6 | F | 5.6 | 62.3 |
| 2 | | | | F | 5.6 | 67.5 | F | 5.7 | 69.5 | F | 5.6 | 79.9 | C | 5.7 | 59.4 |
| 3 | | | | F | 5.6 | 54.3 | F | 5.5 | 78.2 | C | 5.6 | 65.5 | C | 5. | 55.4 |
| 4 | | | | C | 5.7 | 59.3 | C | 5.7 | 65.4 | C | 5.7 | 60.2 | C | 6.1 | 50.9 |
| 5 | | | | C | 5.6 | 65.4 | C | 5.7 | 56.7 | C | 5.9 | 54.4 | C | 6.5 | 44.3 |
| 6 | | | | F | 5.6 | 64.5 | F | 5.5 | 55.6 | F | 5.6 | 61.4 | F | 5.6 | 66.4 |
| 7 | | | | F | 5.6 | 60.6 | F | 5.5 | 66.5 | F | 5.5 | 70.5 | F | 5.6 | 76.5 |

*Appearance testing result: P = free-flowing powder, C = cake
**Moisture testing result, reported in % wt/wt
***Viability testing result, reported in % viability

TABLE 13
Lachnospiraceae

| | T = initial | | | 2 Hr | | | 4 Hr | | | 8 Hr | | | 24 Hr | | |
|---|---|---|---|---|---|---|---|---|---|---|---|---|---|---|---|
| Condition | A* | M** | V*** | A* | M** | V*** | A* | M** | V*** | A* | M** | V*** | A* | M** | V*** |
| 1 | F | 5.0 | 24.4 | F | 4.9 | 24.1 | F | 5.0 | 29.5 | F | 5.0 | 28.6 | F | 5.0 | 23.4 |
| 2 | | | | F | 5.0 | 28.5 | F | 5.0 | 31.2 | F | 5.1 | 27.5 | 0 | 5.2 | 20.5 |
| 3 | | | | F | 5.0 | 27.5 | F | 50.7 | 34.5 | C | 5.2 | 31.5 | C | 5.4 | 21.3 |
| 4 | | | | C | 5.1 | 33.3 | C | 5.2 | 37.6 | C | 5.4 | 33.3 | C | 5.8 | 17.4 |
| 5 | | | | C | 5.2 | 21.4 | C | 5.4 | 29.5 | C | 5.7 | 23.4 | C | 6.2 | 14.5 |
| 6 | | | | F | 5.0 | 29.4 | F | 5.0 | 26.4 | F | 5.0 | 34.7 | F | 5.1 | 29.6 |
| 7 | | | | F | 5.0 | 33.5 | F | 5.0 | 22.5 | F | 5.0 | 26.7 | F | 5.0 | 31.1 |

*Appearance testing result: P = free-flowing powder, C = cake
**Moisture testing result, reported in % wt/wt
***Viability testing result, reported in % viability

TABLE 14
Ruminococcaceae

| | T = initial | | | 2 Hr | | | 4 Hr | | | 8 Hr | | | 24 Hr | | |
|---|---|---|---|---|---|---|---|---|---|---|---|---|---|---|---|
| Condition | A* | M** | V*** | A* | M** | V*** | A* | M** | V*** | A* | M** | V*** | A* | M** | V*** |
| 1 | F | 4.5 | 16.5 | F | 4.5 | 12.3 | F | 4.5 | 19.9 | F | 4.5 | 18.7 | F | 4.6 | 24.3 |
| 2 | | | | F | 4.5 | 17.7 | F | 4.6 | 21 | F | 4.6 | 20.2 | C | 4.8 | 19.4 |
| 3 | | | | F | 4.6 | 20.3 | F | 4.7 | 16.5 | C | 4.8 | 18.9 | C | 5.2 | 12.5 |
| 4 | | | | C | 4.6 | 29.5 | C | 4.8 | 25.4 | C | 5.0 | 14.3 | C | 5.7 | 7.8 |
| 5 | | | | C | 4.8 | 23.4 | C | 5.0 | 14.3 | C | 5.4 | 7.9 | C | 6.2 | 2.3 |
| 6 | | | | F | 4.5 | 27.8 | F | 4.5 | 25.5 | F | 4.5 | 19.9 | F | 4.6 | 21.3 |
| 7 | | | | F | 4.5 | 24.3 | F | 4.5 | 16.6 | F | 4.5 | 21.1 | F | 4.5 | 17.7 |

Appearance testing result: P = free-flowing powder, C = cake
Moisture testing result, reported in % wt/wt
Viability testing result, reported in % viability

TABLE 15
Porphyromonadaceae

| | T = initial | | | 2 Hr | | | 4 Hr | | | 8 Hr | | | 24 Hr | | |
|---|---|---|---|---|---|---|---|---|---|---|---|---|---|---|---|
| Condition | A* | M** | V*** | A* | M** | V*** | A* | M** | V*** | A* | M** | V*** | A* | M** | V*** |
| 1 | F | 5.3 | 34.6 | F | 5.3 | 44.3 | F | 5.3 | 36.5 | F | 5.3 | 49.7 | F | 5.4 | 39.2 |
| 2 | | | | F | 5.4 | 33.1 | F | 5.3 | 47.4 | F | 5.4 | 34.5 | C | 5.5 | 43.3 |
| 3 | | | | F | 5.3 | 29.6 | F | 5.4 | 44.5 | C | 5.4 | 44.5 | C | 5.8 | 38.7 |
| 4 | | | | C | 5.4 | 35.6 | C | 5.6 | 38.9 | C | 5.8 | 29.7 | C | 6.2 | 29.7 |
| 5 | | | | C | 5.6 | 37.8 | C | 5.8 | 33.3 | C | 6.2 | 27.6 | C | 6.7 | 20.2 |
| 6 | | | | F | 5.3 | 39.6 | F | 5.4 | 46.7 | F | 5.4 | 41.1 | F | 5.3 | 37.5 |
| 7 | | | | F | 5.3 | 43.4 | F | 5.4 | 42.3 | F | 5.4 | 39.9 | F | 5.4 | 32.2 |

*Appearance testing result: P = free-flowing powder, C = cake
**Moisture testing result, reported in % wt/wt
***Viability testing result, reported in % viability

The results set forth in this example demonstrate that exposure to increasing humidity levels does lead to an increase in moisture content of the lyophilized material. Moisture absorption is most evident with higher humidity levels and longer exposure times.

From the results, there appears to be a corresponding loss in viability with exposure to increased humidity and duration, associated with increased moisture content of the lyophilized materials. The control samples, both at higher temperature (22° C.) and at recommended storage conditions (−20±5° C.), showed neither an increase in moisture content nor a reduction in viability, suggesting that the impact to product quality is the result of moisture content.

Furthermore, there appears to be a relationship between exposure to elevated humidity and the loss of viability of the organisms. The loss in viability is accompanied by a corresponding shift from free-flowing powder to cake-like appearance, which is consistent with moisture absorption. An increase in moisture values does correlate with a loss in viability, but the change in absolute moisture content (% wt/wt) is actually small, suggesting that the initial moisture level is a critical quality attribute and that the processing environment is a critical process parameter which requires control.

### INCORPORATION BV REFERENCE

This application refers to various issued patents, published patent applications, journal articles, and other publications, all of which are incorporated herein by reference. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

We claim:

1. A composition for preserving anaerobic microorganisms, the composition comprising: a viscosity modifier, and an energy source for the microorganisms, and, when solvated in an aqueous solvent, has a viscosity in the range of about 5 cP to about 6,000 cP, an osmolality in the range of about 300 Osm/L to about 4,000 Osm/L, a pH in the range of about 4 to about 8, and optionally a redox potential in the range of −50 mV to −500 mV.

2. The composition of claim 1, wherein the composition is a cryopreservative composition.

3. A cryopreservative composition for cryopreserving anaerobic microorganisms, the composition comprising: a viscosity modifier, an energy source for the microorganisms, and, when solvated in an aqueous solvent, has a viscosity in the range of about 5 cP to about 6,000 cP, an osmolality in the range of about 300 Osm/L to about 4,000 Osm/L, a pH in the range of about 4 to about 8, and optionally a redox potential in the range of −50 mV to −500 mV.

4. The composition of any one of claims 1-3, further comprising one or more anaerobic microorganisms.

5. The composition of any one of claims 1-4, wherein the viscosity is in the range of about 50 cP to about 4,000 cP, about 100 cP to about 2,000 cP, or about 100 cP to about 1,000 cP.

6. The composition of any one of claims 1-5, wherein the osmolality in the range of about 300 Osm/L to about 3,000 Osm/L, about 400 Osm/L to about 3,000 Osm/L, or about 500 Osm/L to about 3,000 Osm/L.

7. The composition of any one of claims 1-6, wherein the pH is in the range of about 4 to about 8, about 5 to about 8, about 4 to about 7.5, about 5 to about 7.5, or about 5.5 to about 7.5.

8. The composition of any one of claims 1-7, wherein the composition comprises about 0.01% (w/w) to about 0.4% (w/w) of the viscosity modifier, when solvated in an aqueous solvent.

9. The composition of any one of claims 1-8, wherein the viscosity modifier is a polymeric composition.

10. The composition of claim 9, wherein the polymeric composition comprises an alginate, acacia, alginic acid, an agar, alamic acid, bentonite, carbomer, a carrageenan, a cellulose, a dextrin, a gelatin, gellan gum, guar gum, a carboxymethylcellulose, a hydroxyethylcellulose, a hydroxypropylcellulose, a methyl cellulose, a mucin, pectin, a starch, xanthan gum or a combination thereof.

11. The composition of claim 10, wherein the polymeric composition comprises an alginate.

12. The composition of claim 11, wherein the alginate is sodium alginate.

13. The composition of claim 12, wherein the sodium alginate has a viscosity range of about 250 cP to about 14,000 cP.

14. The composition of any one of claims 1-13, wherein the energy source is a cell culture media that permits growth of the microorganisms.

15. The composition of any one of claims 1-14, wherein the composition comprises about 3% (w/w) to about 15% (w/w) of the energy source, when solvated in an aqueous solvent.

16. The composition of any one of claims 1-15, wherein the energy source is a monosaccharide, a disaccharide, an oligosaccharide, or a combination thereof.

17. The composition of claim 16, wherein the monosaccharide is selected from the group consisting of glucose, fructose, xylose, and combinations thereof.

18. The composition of claim 16, wherein the disaccharide is trehalose, sucrose, or a combination thereof.

19. The composition of claim 16, wherein the oligosaccharide is an inulin derivative.

20. The composition of claim 17, wherein the inulin derivative is a fructo-oligosaccharide.

21. The composition of any one of claims 1-20, further comprising a protein stabilizer.

22. The composition of claim 21, wherein the composition comprises about 5% (w/w) to about 10% (w/w) of the protein stabilizer, when solvated in an aqueous solvent.

23. The composition of claim 21 or 22, wherein the protein stabilizer is selected from the group consisting of an amino acid, an antioxidant, a disaccharide, a reducing agent, polyethylene glycol, and combinations thereof.

24. The composition of claim 23, wherein the disaccharide is selected from the group consisting of trehalose, sucrose, isomaltose, lactose, maltose, lactulose, cellobiose, and combinations thereof.

25. The composition of any one of claims 4-24, wherein the one or more anaerobic microorganisms are present in the form of a cell paste or cell slurry.

26. The composition of claim 25, wherein the composition comprises about 30% (w/w) to about 50% (w/w) of the cell paste or cell slurry, when solvated in an aqueous solvent.

27. A cryopreservative composition for cryopreserving anaerobic microorganisms, the composition comprising:
(i) about 30% (w/w) to about 50% (w/w) of a cell paste, wherein the cell paste comprises one or more anaerobic microorganisms;
(ii) about 3% (w/w) to about 15% (w/w) of an energy source for the microorganisms;
(iii) about 0.01% (w/w) to about 0.4% (w/w) of a viscosity modifier; and
(iv) about 30% (w/w) to about 60% (w/w) of an aqueous solvent,
wherein the cryopreservative composition has a viscosity in the range of about 5 cP to about 6,000 cP, an osmolality in the range of about 300 Osm/L to about 4,000 Osm/L, and a pH in the range of about 4 to about 8.

28. A cryopreservative composition for cryopreserving anaerobic microorganisms, the composition comprising:
(i) about 30% (w/w) to about 50% (w/w) of a cell paste, wherein the cell paste comprises one or more anaerobic microorganisms;
(ii) about 0.5% (w/w) to about 5% (w/w) raftilose;
(iii) about 3% (w/w) to about 10% (w/w) maltodextrin;
(iv) about 0.01% (w/w) to about 0.4% (w/w) sodium alginate;
(v) about 5% (w/w) to about 10% (w/w) trehalose;
(vi) 0% (w/w) to about 5% (w/w) sucrose; and
(vii) about 35% (w/w) to about 55% (w/w) water,
wherein the cryopreservative composition has a viscosity in the range of about 5 cP to about 6,000 cP, an osmolality in the range of about 300 Osm/L to about 4,000 Osm/L, and a pH in the range of about 4 to about 8.

29. The composition of claim 27 or 28, wherein the composition has a redox potential in the range of −50 mV to −500 mV.

30. The composition of any one of claims 1-29, further comprising a buffering agent.

31. A preserved composition comprising: one or more anaerobic microorganisms, a viscosity modifier, an energy source for the microorganism, a redox potential in the range of −50 mV to −500 mV, and a water content of less than 5% (w/w).

32. The composition of claim 31, wherein the composition is a cryopreserved composition.

33. A cryopreserved composition comprising: one or more anaerobic microorganisms, a viscosity modifier, an energy source for the microorganism, a redox potential in the range of −50 mV to −500 mV, and a water content of less than 5% (w/w).

34. The composition of any one of claims 31-33, wherein the water content is in the range of about 2% (w/w) to about 4% (w/w) or about 2% (w/w) to about 3% (w/w).

35. The composition of any one of claims 31-34, wherein the viscosity modifier is a polymeric composition.

36. The composition of claim 35, wherein the polymeric composition comprises an alginate, acacia, alginic acid, an agar, alamic acid, bentonite, carbomer, a carrageenan, a cellulose, a dextrin, a gelatin, gellan gum, guar gum, a carboxymethylcellulose, a hydroxyethylcellulose, a hydroxypropylcellulose, a methyl cellulose, a mucin, pectin, a starch, xanthan gum or a combination thereof.

37. The composition of claim 35 or 36, wherein the polymeric composition comprises an alginate.

38. The composition of claim 36, wherein the alginate is sodium alginate.

39. The composition of claim 38, wherein the sodium alginate has a viscosity range from 250 to 14,000 cP.

40. The composition of any one of claims 31-39, wherein the energy source is a cell culture media that permits growth of the microorganisms.

41. The composition of any one of claims 31-40, wherein the energy source is a monosaccharide, a disaccharide, an oligosaccharide, or a combination thereof.

42. The composition of claim 41, wherein the monosaccharide is selected from the group consisting of glucose, fructose, xylose, and combinations thereof.

43. The composition of claim 41, wherein the disaccharide is selected from the group consisting of trehalose, isomaltose, lactose, maltose, lactulose, cellobiose, sucrose, and combinations thereof.

44. The composition of claim 41, wherein the oligosaccharide is an inulin derivative.

45. The composition of claim 44, wherein the inulin derivative is a fructo-oligosaccharide.

46. The composition of any one of claims 29-45, further comprising a protein stabilizer.

47. The composition of claim 46, wherein the protein stabilizer is selected from the group consisting of an amino acid, an antioxidant, a disaccharide, a reducing agent, polyethylene glycol, and combinations thereof.

48. The composition of claim 47, wherein the disaccharide is selected from the group consisting of trehalose, sucrose, isomaltose, lactose, maltose, lactulose, cellobiose, and combinations thereof.

49. The composition of any one of claims 31-48, further comprising a buffering agent.

50. A cryopreserved composition comprising:
(i) one or more anaerobic microorganisms;
(ii) an energy source comprising raftilose and maltodextrin;
(iii) sodium alginate;
(iv) a protein stabilizer comprising trehalose, sucrose, and combinations thereof and (vi) a water content of less than 5% (w/w).

51. The composition of claim 50, where the composition has a redox potential in the range of −50 mV to −500 mV.

52. The composition of any one of claims 1-51, wherein the one or more anaerobic microorganisms is selected from the group consisting of Akkermansiaceae, Bacillaceae, Bacteroidaceae, Bifidobacteriaceae, Christensenellaceae, Clostridiaceae, Coriobacteriaceae, Eggerthellaceae, Enterobacteriaceae, Eubacteriaceae, Lachnospiraceae, Lactobacillaceae, Leuconostocaceae, Odoribacteraceae, Oxalobacteraceae, Porphyromonadaceae, Prevotellaceae, Rikenellaceae, Ruminococcaceae, Streptococcaceae, and combinations thereof.

53. The composition of any of claims 31-52, wherein the composition, when stored at 4° C. for 6 months or 12 months and then rehydrated results in a loss of colony forming units (CFUs) by less than 3, 2, 1 or 0.5 log.

54. The composition of any one of claims 31-52, wherein the composition remains substantially stable at 4° C. for about 2 years.

55. The composition of any one of claims 31-52, wherein the composition, when rehydrated in an aqueous fluid, permits regrowth of the one or more microorganisms within 5, 4, 3, or 2 hours of rehydration.

56. A pharmaceutical composition comprising the cryopreserved composition of any one of claims 31-55, optionally disposed within a capsule, tablet or caplet.

57. A food product or probiotic comprising the cryopreserved composition of any one of claims 31-55, optionally disposed within a capsule, tablet or caplet.

58. A method of modulating the gastrointestinal microbiome of a subject in need thereof, the method comprising administering to the subject an effective amount of a composition of any one of claims 31-55, the pharmaceutical composition of claim 56, or the food product or probiotic of claim 57.

59. A method of producing the cryopreserved composition of any one of claims 31-55, the method comprising the steps of:
(a) combining anaerobic microorganisms with an excipient blend comprising an aqueous mixture comprising a viscosity modifier, and an energy source for the microorganisms to produce a cryopreservative mixture, wherein the cryopreservative mixture has a viscosity in the range of about 5 cP to about 6,000 cP, an osmolality in the range of about 300 Osm/L to about 4,000 Osm/L, a pH in the range of about 4 to about 8, and optionally a redox potential in the range of −50 mV to −500 mV; and
(b) freeze drying the cryopreservative mixture to reduce the water content of the cryopreservative mixture to less than about 5% (w/w) to produce the cryopreserved composition.

60. The method of claim 59, wherein, in step (a), the microorganisms are in the form of a cell paste.

61. The method of claim 59 or 60, wherein the cell paste comprises the microorganisms in cell culture media.

62. The method of any one of claims 58-60, further comprising packaging the cryopreserved composition in a capsule, tablet or caplet.
